(19) United States
(12) Patent Application Publication
(10) Pub. No.: US 2026/0275292 A1
(43) Pub. Date: Sep. 17, 2026

(54) MATURATION MEDIUM COMPOSITIONS AND METHODS FOR HUMAN HEART ORGANOID MATURATION

(71) Applicant: Board of Trustees of Michigan State University, East Lansing, MI (US)

(72) Inventors: Aitor AGUIRRE, Okemos, MI (US); Brett VOLMERT, Lansing, MI (US)

(73) Assignee: Board of Trustees of Michigan State University, East Lansing, MI (US)

(21) Appl. No.: 18/868,539

(22) PCT Filed: Jul. 19, 2023
(86) PCT No.: PCT/US2023/028099

§ 371 Date: Nov. 22, 2024

Related U.S. Application Data
(60) Provisional application No. 63/391,452, filed on Jul. 22, 2022.
(60) Provisional application No. 63/432,565, filed on Dec. 14, 2022.

(51) Int. Cl. C12N 5/077

(52) U.S. Cl. CPC C12N 5/0657; C12N 2500/34; C12N 2500/36; C12N 2500/38; C12N 2501/105; C12N 2501/305; C12N 2501/33; C12N 2506/45

(57) ABSTRACT

A maturation medium and a method for maturing an early embryonic human heart organoid into a mature human heart organoid are provided herein. The maturation medium includes a cell growth medium containing a medium supplement including one or more fatty acid, triiodothyronine (T3) growth hormone, insulin, one or more antioxidant, a sugar, and carnitine; one or more additional fatty acid; an additional carnitine or creatine; and an additional T3 growth hormone. The maturation medium may further include one or more of an additional sugar, an additional antioxidant, and a growth factor. The method includes contacting an early embryonic human heart organoid with one or more maturation medium to produce a mature human heart organoid.

### CROSS REFERENCE TO RELATED PATENT APPLICATIONS

This patent application claims the benefit of U.S. Provisional Patent Application No. 63/391,452 filed on 22 Jul. 2022 and U.S. Provisional Patent Application No. 63/432,565 filed on 14 Dec. 2022. The entire contents of each patent application recited above is hereby incorporated by reference.

### GOVERNMENT RIGHTS

This invention was made with government support under HL135464 and HL151505 awarded by the National Institute of Health and National Heart, Lung and Blood Institute. The government has certain rights in the invention.

### FIELD

The present disclosure relates to a maturation medium and a method and model for the high throughput generation of mature human heart organoids, such as fetal-like human heart organoids, which may be further matured to more adult-like human heart organoids.

### BACKGROUND

Cardiovascular diseases (CVDs), including disorders of the heart and blood vessels, are the leading causes of death globally, contributing to an estimated 17.9 million deaths annually¹. Laboratory models of the heart are used to better understand the etiology and mechanisms of CVDs in high detail. Several model systems to research CVDs are used, ranging from primary and induced pluripotent stem cell (iPSC)-derived cardiomyocyte cultures to animal models and 3D-culture systems, such as spheroids and engineered heart tissues²⁻⁷. Nevertheless, many of these systems fail to fully recapitulate the complex nature of the human heart due to a variety of reasons, including the absence of endogenous extracellular matrix (ECM) and non-cardiomyocyte cardiac cell types, as well as the lack of physiological morphology and cellular organization^{8,9}. In addition, animal models possess distinct, non-human physiology, metabolism, electrophysiology and pharmacokinetic profiles which often do not predict human-relevant responses^{8,9} accurately. Thus, these systems and methods are unsuitable for comprehensively investigating and modeling human disease and physiology.

The introduction of human-relevant models is paramount to the discovery of effective, clinically translatable solutions to CVDs. Over the last ten years, advances in human induced pluripotent stem cell (hiPSC)¹⁰⁻¹² and organoid^{13,14} technologies have advanced techniques to better model and study human systems with increasing precision. Recently, methodologies to create human heart organoids from pluripotent stem cells have been reported. These methods enable the study of human heart development and disease¹⁵⁻¹⁹ in a dish to a degree unseen before due to their cellular complexity and physiological relevance. Yet, these systems still fall short of recapitulating important aspects of human heart development and the late embryonic human heart, such as anterior-posterior patterning, coronary vascularization and lack important cell populations contributing to heart structure (e.g., neural crest).

Furthermore, the metabolic transition from glycolysis to fatty acid oxidation is a paramount step in the late stages of cardiac development, preparing the heart for increased energy expenditure as well as inducing transcriptional regulation and stimulating physiological maturation^{28,30,95-97,141} Efforts have been pursued to simulate these phenomena in vitro with cardiomyocytes and engineered heart tissues and have found beneficial effects from modified glucose concentrations and the addition of fatty acids^{7,20,21,23,142} However, these systems are simplistic models and do not possess the high physiological complexity as observed in human heart organoids.

Collectively, there exists a pressing need to develop more mature and sophisticated in vitro model systems for investigating human heart development and disease pathology.

### SUMMARY OF THE INVENTION

This section provides a general summary of the disclosure and is not a comprehensive disclosure of its full scope or all of its features.

Provided herein is a maturation medium including a cell growth medium containing a medium supplement including one or more fatty acid, triiodothyronine (T3) growth hormone, insulin, one or more antioxidant, a sugar, and carnitine. The maturation medium further includes one or more additional fatty acid, an additional carnitine or creatine, and an additional T3 growth hormone.

In another embodiment, a method for maturing an early embryonic human heart organoid into a mature human heart organoid is provided. The method includes contacting the early embryonic human heart organoid with a maturation medium. The maturation medium includes a cell growth medium containing a medium supplement including one or more fatty acid, triiodothyronine (T3) growth hormone, insulin, one or more antioxidant, a sugar, and carnitine. The maturation medium further includes one or more additional fatty acid, an additional carnitine or creatine, and an additional T3 growth hormone.

In another embodiment, a mature human heart organoid produced by the methods described herein is provided.

Other embodiments, including particular aspects of the embodiments summarized above, will be evident from the detailed description that follows.

### BRIEF DESCRIPTION OF THE DRAWINGS

FIGS. 1A-1H. Developmental induction methods for improving human heart organoid developmental modeling. FIG. 1A is a schematic diagram depicting the differentiation protocol for creating human heart organoids and the media conditions for the four maturation strategies (Control, MM, EMM1, and EMM2/1). FIG. 1B are Brightfield images of organoids throughout the 30-day culture period. Two representative organoids are shown for each condition (data representative of 23-24 organoids per condition). Scale bar=400 μm. FIG. 1C is a quantification of organoid long diameter and short diameter at day 30 of culture for each maturation strategy (n=7-8 organoids per condition). Data presented as mean±s.e.m. FIG. 1D is a quantification of organoid area at day 30 of organoid culture for each maturation strategy (n=7-8 organoids per condition across two independent experiments). Data presented as a violin plot with all points. Data presented as a violin plot with all points. FIG. 1E is a quantification of percentage of organoids visibly beating under brightfield microscopy in each condition from 5 different organoid batches (n=22-24 organoids in each condition across five independent experiments). FIG. 1F are TEM images displaying sarcomeres, myofibrils (M) and I-bands (arrows) in day 15 organoids and in organoids from each maturation condition at day 30 (n=4 organoids per condition). Scale bars=1 μm. FIG. 1G is a quantification of sarcomere length within TEM images. Data presented as mean±s.e.m (n=4 organoids per condition). One-way ANOVA with Brown-Forsythe and Welch multiple comparisons tests. FIG. 1H is an mRNA expression of key sarcomeric genes involved in cardiomyocyte maturation between days 20 and 30 of culture for each condition (n=7-14 organoids per day per condition per gene across three independent experiments). Data presented as log₂ fold change normalized to Day 20. Values=mean±s.e.m.

FIGS. 2A-2D. Single-cell RNA sequencing of human heart organoids reveals distinct cardiac cell populations. FIG. 2A are UMAP projections of k-means clustering of single-cell RNA sequencing data for each condition in day 34 organoids. Cluster identities are located in the legend below. FIG. 2B is a quantification of total cell count percentages per cluster. Colors of regions correspond to those found in the legend in FIG. 2A. FIG. 2C is a differential expression heatmap displaying the top 10 differentially expressed genes for all clusters. FIG. 2 D are feature plots displaying key marker genes for each cluster. Color intensity represents the relative value of gene expression.

FIGS. 3A-3B. Cluster identity and cell-cell communication networks highlight the importance of self-organization in heart organoid development. FIG. 3A is a dot plot of differentially expressed genes in each cluster for each condition. Color is indicative of the average expression level across all cells, and the size of the circle is indicative of the percentage of cells within a particular cluster that express the respective gene. FIG. 3B is a visualization of cell-cell ligand-receptor communication networks for each condition. Colors of clusters (exterior) matches that of UMAP projections. Ligands are indicated as blue bands and receptors are indicated by red bands. Arrows within depict pairing from ligands to receptors.

FIGS. 4A-4K. Human heart organoids develop increasingly mature metabolic profiles following developmental induction conditions. FIG. 4A is mitochondrial labeling within day 30 human heart organoids in each condition (n=6 organoids per condition). White=Mitotracker, blue=NucBlue. Scale bars=10 μm. Detailed images of mitochondria are shown below each main image. FIG. 4B is quantification of mitochondrial area surrounding each individual nucleus (n=6 organoids per condition, n=50-70 measurements per condition). Values=mean±s.e.m., one-way ANOVA with Brown-Forsythe and Welch multiple comparisons tests. FIG. 4C are TEM images displaying mitochondria in day 15 organoids and in organoids from each maturation condition at day 30 (n=4 organoids per condition). Yellow arrows indicate mitochondria, LD=lipid droplets, Gg=glycogen granules. Scale bars=1 μm. FIG. 4D is quantification of mitochondrial area from TEM images. Values=mean±s.e.m., one-way ANOVA with Brown-Forsythe and Welch multiple comparisons tests (n=4 organoids per condition, n=40-144 mitochondria measured per condition). FIG. 4E is mRNA expression of metabolic genes PPARGC1A and CPT1B between days 20 and 30 of culture for each condition (n=8 organoids per condition across three independent experiments). Data presented as log 2 fold change normalized to Day 20. Values=mean±s.e.m. FIG. 4F are oxygen consumption rate measurements from Agilent Seahorse XFe96 metabolic stress test assay in all conditions (n=8 organoids per condition across two independent experiments). Values=mean±s.e.m. FIG. 4G is quantifications from oxygen consumption rate assay (n=8 organoids per condition across two independent experiments) for basal respiration. FIG. 4H is quantifications from oxygen consumption rate assay (n=8 organoids per condition across two independent experiments) for maximal respiration. FIG. 4I is quantifications from oxygen consumption rate assay (n=8 organoids per condition across two independent experiments) for spare respiratory capacity. FIG. 4J are feature plots displaying key metabolic genes upregulated in the VCM and ACM clusters. Color intensity represents the relative value of gene expression per gene. FIG. 4K are expression heatmaps of key metabolic genes in the VCM and ACM clusters in each condition. Data displayed as log 2 fold change and normalized to each column (for each gene).

FIGS. 5A-5I. Developmental induction conditions promote progressive electrophysiological maturation in human heart organoids. FIG. 5A are representative calcium transient traces within day 30 human heart organoids from each condition (n=12 organoids per condition across three independent experiments). Traces represent data from an individual cardiomyocyte within human heart organoids. Additional traces are shown in FIG. 10A. FIG. 5B is quantification of peak amplitude of calcium transient traces from each condition (n=12 organoids per condition across three independent experiments). A minimum of 2 regions and 16 peaks were quantified and averaged for each organoid. Values=mean±s.e.m. FIG. 5C is quantification of calcium transient peak frequency for each condition (n=12 organoids per condition across three independent experiments). A minimum of 2 regions and 16 peaks were quantified and averaged for each organoid. Values=mean±s.e.m. FIG. 5D are feature plots displaying key electrophysiological genes differentially expressed in the VCM and ACM clusters in each condition. Color intensity represents the relative value of gene expression per gene. FIG. 5E is mRNA expression of key electrophysiological genes between days 20 and 30 of culture for each condition (n=8 organoids per day per condition per gene across three independent experiments). Data presented as log₂ fold change normalized to Day 20. Values=mean±s.e.m. FIG. 5F are representative voltage tracings of organoids in the EMM2/1 and Control conditions depicting atrial-, nodal-, and ventricular-like action potentials (n=9 individual cells from 3 independent organoids per action potential subtype per condition across three independent experiments). FIG. 5G are representative immunofluorescence images of caveolin-3 puncta within TNNT2+ regions in organoids for each condition (n=15 organoids per condition across three independent experiments). Green=caveolin-3, red=TNNT2, blue=DAPI. Scale bar=20 km. FIG. 5H are quantification of the caveolin-3 positive area per 400 square m for each condition from images presented in FIG. 5G (n=15 organoids per condition across three independent experiments). Data presented as fold change normalized to control. Values=mean±s.e.m., one-way ANOVA with Brown-Forsythe and Welch multiple comparisons tests. FIG. 5I are representative immunofluorescence images of KCNJ2+ puncta within TNNT2+ regions in organoids for each condition (n=14 organoids per condition across 3 independent experiments). KCNJ2=green, TNNT2=red, DAPI=blue. Scale bar=20 km. FIG. 5J is quantification of the total number of KCNJ2+ puncta for each condition from images presented in FIG. 5I (n=14 organoids per condition across 3 independent experiments). Data presented as fold change normalized to Control. Values=mean±s.e.m., one-way ANOVA with Dunnett's multiple comparisons tests.

FIGS. 6A-6J. Developmental induction promotes the emergence of a proepicardial organ and formation of distinct atrial and ventricular chambers by self-organization. FIG. 6A are representative surface and interior immunofluorescence images of individual day 30 organoids in all conditions displaying WT1 (green), TNNT2 (red), and DAPI (blue). Three organoids are displayed for each condition (n=12-15 organoids per condition across two independent experiments). Scale bars=200 μm. FIG. 6B is quantification of TNNT2+ chamber area in each condition from images presented in FIG. 6A (n=12-15 organoids per condition across two independent experiments). Values are presented as fold change normalized to Control. Values=mean±s.e.m., one-way ANOVA with Dunnett's multiple comparisons test. FIG. 6C is quantification of WT1+ chamber area in each condition from images presented in FIG. 6A (n=12-15 organoids per condition across two independent experiments). Values are presented as fold change normalized to Control. Values=mean±s.e.m., one-way ANOVA with Dunnett's multiple comparisons test. FIG. 6D are representative surface and interior immunofluorescence images of individual day 30 organoids in all conditions displaying MYL2 (green), MYL7 (red), and DAPI (blue). Three organoids are displayed for each condition (n=13 organoids per condition across three independent experiments). Scale bars=200 μm. FIG. 6E is quantification of MYL2+ area in each organoid in each condition from images presented in FIG. 6D (n=9-13 organoids per condition across three independent experiments). Values are presented as fold change normalized to Control. Values=mean±s.e.m., one-way ANOVA with Dunnett's multiple comparisons test. FIG. 6F are representative immunofluorescence images of individual day 30 organoids in all conditions displaying NR2F2 (green), MYL7 (red), and DAPI (blue). Three organoids are displayed for each condition (n=12 organoids per condition across three independent experiments). Scale bars=200 μm. FIG. 6G is quantification of colocalization (Pearson's coefficient) between NR2F2 (green) and MYL3 (red) from images presented in FIG. 6F Values=mean±s.e.m., unpaired t-test. FIG. 6H are feature plot highlighting the VCM and ACM clusters for further use in FIGS. 6I and 6J. FIG. 6I are feature plots displaying hallmark atrial chamber identity genes that are differentially expressed in the ACM cluster. Color intensity represents the relative value of gene expression per gene. FIG. 6J are feature plots displaying ventricular chamber identity genes that are differentially expressed in the VCM cluster. Color intensity represents the relative value of gene expression per gene.

FIGS. 7A-7J. An endogenous retinoic acid gradient is responsible for spontaneous anterior-posterior heart tube patterning. FIG. 7A is a schematic portraying in utero cardiac heart tube formation, highlighting the localization and intensity of the retinoic acid gradient from the anterior (arterial pole) to the posterior (venous pole) of the primitive heart tube. FIG. 7B are Raman spectroscopy intensity plots for organoids from all four developmental maturation conditions at day 30 of culture. Peaks of interest are marked, such as DNA, cardiac troponin, and retinoic acid. Data presented is representative of n=3 organoids per condition. FIG. 7C is mRNA expression of ALDH1A2 in all conditions at day 30 (n=7 organoids per condition across two independent experiments). Data presented as log 2 fold change normalized to Control. Values=mean±s.e.m., one-way ANOVA with Dunnett's multiple comparisons test. FIG. 7D is a feature plot displaying expression of ALDH1A2. Color intensity represents the relative value of gene expression. FIG. 7E are representative immunofluorescence images of individual day 30 organoids in all conditions displaying ALDH1A2 (green), TBX18 (red), and DAPI (blue). Three organoids are displayed for each condition (representative of n=22-24 organoids per condition across three independent experiments). Scale bar=200 μm. FIG. 7F are high magnification images of EMM2/1 and Control organoids shown in FIG. 7C, displaying ALDH1A2 (green), TBX18 (red), and DAPI (blue). Yellow square in image at top (Scale bar=200 μm) represents area of high magnification. Scale bar=50 μm. FIG. 7G is quantification of ALDH1A2⁺TBX18⁺ area within organoids in each condition from images presented in FIG. 7E (n=22-24 organoids per condition across three independent experiments). Data presented as fold change normalized to Control. Values=mean±s.e.m., one-way ANOVA with Dunnett's multiple comparisons test. FIG. 7H are representative immunofluorescence images of individual day 30 EMM2/1 organoids following exposure to either deoxyaminobenzaldehyde (DEAB), retinoic acid (RA), or no treatment (Untreated). Staining was performed for MYL3 (pink), NR2F2 (green), and DAPI (blue). Two organoids are displayed for each condition (n=9 organoids per condition across two independent experiments). Scale bar=200 μm. FIG. 7I is quantification of NR2F2+ area of organoids presented in FIG. 7H (n=9 organoids per condition across two batches of organoids). Values=mean±s.e.m., one-way ANOVA with Dunnett's multiple comparisons test. FIG. 7J is quantification of MYL3+ area of organoids presented in FIG. 7H (n=9 organoids per condition across two batches of organoids). Values=mean±s.e.m., one-way ANOVA with Dunnett's multiple comparisons test.

FIGS. 8A-8J. Heart organoid treatment with Ondansetron models morphological and electrophysiological phenotypes of congenital heart disease. FIG. 8A are representative immunofluorescence images of individual day 30 EMM2/1 organoids following exposure to varying concentrations of ondansetron (1 μM, 10 μM, or 100 μM) or no treatment (Untreated) from day 9 to day 30 of culture. Staining was performed for MYL2 (green), MYL7 (red), and DAPI (blue). Three organoids are displayed for each condition (n=12 organoids per condition across two independent experiments). Scale bar=200 μm. FIGS. 8B-8C are quantification of MYL2+ area and MYL7+ area, respectively, for each condition from images presented in FIG. 8A (n=12 organoids per condition across two independent experiments). Data presented as fold change normalized to Untreated. Values=mean±s.e.m., one-way ANOVA with Dunnett's multiple comparisons test. FIG. 8D is mRNA expression of MYL2 in all conditions at day 30 (n=6 organoids per condition across two independent experiments). Data presented as log 2 fold change normalized to Control. Values=mean±s.e.m., one-way ANOVA with Dunnett's multiple comparisons test. FIGS. 8E-6F are representative voltage tracings of organoids showing three voltage traces from independent organoids, respectively, in each condition (n=6 organoids per condition across two independent experiments). FIGS. 8G-J are quantification of voltage tracings from individual organoids in each condition from traces presented in FIGS. 8E-8F (n=6 organoids per condition across two independent experiments), displaying frequency, amplitude, APD30, and APD90, respectively. Values=mean±s.e.m., one-way ANOVA with Dunnett's multiple comparisons test.

FIGS. 9A-9C. Longitudinal assessment of apoptosis across all maturation conditions. FIG. 9A are representative fluorescence images of day 20 and day 30 organoids from each condition displaying FlipGFP fluorescence signal (n=12 organoids per condition per day). FIG. 9B are representative fluorescence images of day 30 EMM2/1 organoids following a 48-hour exposure to doxorubicin displaying FlipGFP fluorescence signal (n=6 organoids). FIG. 9C is quantification of fluorescence intensity from images presented in FIG. 9A (n=12 organoids per condition per day). Data presented as fold change normalized to day 20. Values=mean±s.e.m., matched two-way ANOVA with Tukey's multiple comparisons test.

FIGS. 10A-10C. Transcriptomic organoid landscape reveals similarities to in vivo developing human hearts. FIG. 10A is a schematic portraying comparisons in timelines of embryonic heart development and human heart organoid development. FIG. 10B are UMAP projections displaying human embryonic heart and human heart organoid scRNAseq datasets. Cluster naming for (Asp et al, Cell, 2019) is preserved from original text. Cluster identity and color for the human heart organoid dataset is preserved from that shown in (FIG. 2A). FIG. 10C is a PCA plot for datasets presented in FIG. 10B.

FIGS. 11A-11F. Human heart organoids share key gene expression with embryonic human hearts across cardiac cell types. FIGS. 11A-11F are each feature plots displaying key marker genes for each cluster in the Asp 2019, Cui 2019, and human heart organoid datasets for the following respective clusters: Atrial Cardiomyocytes; Ventricular Cardiomyocytes; Proepicardial-derived Cells; Epicardial Cells; Valve Cells; and Conductance Cells. Color intensity represents the relative value of gene expression.

FIG. 12A. Calcium measurement displays reproducibility across independent organoids. FIG. 12A are representative calcium transient traces within day 30 human heart organoids from each condition (n=12 organoids per condition across three independent experiments). Data from 6 independent organoids are displayed. Traces represent data from an individual cardiomyocyte within human heart organoids.

FIGS. 13A-13D. Ventricular and atrial chamber formation is reproducible across three hPSC lines. FIG. 13A are representative immunofluorescence images of individual day 30 organoids in both Control and EMM2/1 conditions from the cell lines L1, BYS0111, and H9 displaying NR2F2 (green), MYL3 (red), and DAPI (blue). Three organoids are displayed per condition per cell line (n=12 organoids per condition across three independent experiments for L1 organoids; n=11 organoids per condition across two independent experiments for BYS0111 organoids; n=12 organoids per condition across two independent experiments for H9 organoids). Scale bars=200 μm. FIGS. 13B-13D are quantification of colocalization (Pearson's coefficient) between NR2F2 (green) and MYL3 (red) from images presented in FIG. 13A for L1, BYS0111, and H9 organoids, respectively. Values=mean±s.e.m., unpaired t-test.

FIGS. 14A-14C. Live longitudinal imaging by optical coherence tomography reveals large, interconnected chambers within human heart organoids. FIG. 14A is a schematic of custom-built optical coherence tomography (OCT) system for human heart organoid imaging. FIG. 14B are longitudinal OCT cross-sectional scans of human heart organoids from day 20 to day 30 in each condition. Scale bars=500 μm. Images shown represent 6 organoids per condition. FIG. 14C are 3D segmentation of OCT scans from images presented in FIG. 14A reveal the temporally dynamic volumetric visualization of chamber identity in each condition.

FIGS. 15A-15D. Endothelial cell localization and morphology is perturbed through enhanced developmental maturation strategies. FIG. 15A are representative immunofluorescence images from the surface and interior of day 30 organoids with DAPI (blue), TNNT2 (red), and PECAM1 (green) in each condition (n=7-8 organoids per condition across two independent experiments). Scale bars=200 μm. FIG. 15B are representative day 30 organoid immunofluorescence images with DAPI (blue), TNNT2 (red), and PECAM1 (green) from images presented in FIG. 15A (n=7-8 organoids per condition across two independent experiments). Images presented as maximum intensity projections. Scale bars=200 μm. FIG. 15C is a quantification of PECAM1+ area presented in FIG. 15B (n=7-8 organoids per condition across two independent experiments). Data presented as log fold change normalized to Control. Values=mean±s.e.m., one-way ANOVA with Dunnett's multiple comparisons test. FIG. 15D are representative high magnification immunofluorescence images of organoids in each condition with DAPI (blue), TNNT2 (red), and PECAM1 (green) (n=7-8 organoids per condition across two independent experiments). Scale bars=50 μm. The images on top are representative low magnification organoids (Scale bar=200 μm) for each condition with the yellow square representing area of high magnification. Images presented as maximum intensity projections.

FIGS. 16A-16D. Emergence of an ALDH1A2+ proepicardial pole through using the EMM2/1 developmental maturation strategy is reproducible across three hPSC lines. FIG. 16A are representative immunofluorescence images of individual day 30 organoids in both Control and EMM2/1 conditions from the cell lines L1, BYS0111, and H9 displaying ALDH1A2 (green), TBX18 (red), and DAPI (blue). Three organoids are displayed per condition per cell line (n=22-24 organoids per condition across three independent experiments for L1 organoids; n=12 organoids per condition across two independent experiments for BYS0111 organoids; n=12 organoids per condition across two independent experiments for H9 organoids). Scale bars=200 m. FIGS. 16B-16D are each a quantification of ALDH1A2⁺TBX18⁺ area within organoids in each condition from images presented in FIG. 16A for L1, BYS0111, and H9 organoids, respectively. Data presented as fold change normalized to Control. Values=mean±s.e.m., unpaired t-test.

FIGS. 17A-17G. Transcriptional profiles for key genes within human heart organoids are reproducible across three hPSC lines. FIGS. 17A-17G each are mRNA expression of select genes in the Control and EMM2/1 conditions from L1, BYS0111 and H9 organoids at day 30 (n=7-14 organoids per condition across three independent experiments for L1 organoids; n=8 organoids per condition across two independent experiments for BYS0111 organoids; n=8 organoids per condition across two independent experiments for H9 organoids). FIG. 17A is mRNA expression of MYL2 gene. FIG. 17B is mRNA expression of MYL7 gene. FIG. 17C is mRNA expression of MYH6 gene. FIG. 17D is mRNA expression of MYH7 gene. FIG. 17E is mRNA expression of ALDH1A2 gene. FIG. 17F is mRNA expression of PPARGC1A gene. FIG. 17G is mRNA expression of WT1 gene. Data presented as log 2 fold change normalized to Control for each cell line. Values=mean±s.e.m., unpaired t-test.

FIGS. 18A-18C. Apoptosis is not a contributing factor towards Ondansetron-induced heart organoid malformations. FIG. 18A are representative fluorescence images of day 30 EMM2/1 organoids from each condition displaying FlipGFP fluorescence signal following Ondansetron treatment from day 9 to day 30 and Doxorubicin treatment from day 28 to day 30 (across two independent experiments each: n=12 organoids per condition for Ondansetron group; n=6 organoids for Doxorubicin group). Scale bars=200 μm. FIG. 18B is a quantification of fluorescence intensity from images presented in FIG. 18A. Data presented as fold change normalized to Untreated. Values=mean±s.e.m., one-way ANOVA with Games-Howell's multiple comparisons. FIG. 18C is a quantification of percentage of beating organoids throughout treatment period for each Ondansetron condition from day 0 to day 30 (n=23-24 organoids per condition across two independent experiments).

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

As discussed above, laboratory models of the human heart have made considerable progress over the last decades, beginning with animal models and primary cardiomyocyte culture and moving onwards to induced pluripotent stem cell-derived cardiac tissues (e.g. cardiomyocytes) and tissue engineering approaches (3D printing, biomaterials). The latest advances in human heart models are heart organoids generated from pluripotent stem cells^{15-17,115}. However, these systems do not possess the true complexity of the in utero human heart, owing to a lack of maturity and faithfulness to human physiology, morphology, cellular organization, and functionality. These shortcomings severely limit the scope of relevance of traditional model systems.

Thus, there is a need for a developmentally and physiologically relevant model of an adult human heart, for example, from hiPSCs, which can be achieved in an efficient and high throughput fashion. Provided herein are maturation mediums and methods for generating a mature human heart organoid in a reproducible and high-throughput fashion with developmental induction strategies inspired by in utero biological steps, to produce human heart organoids with higher anatomical complexity and physiological relevance along first trimester fetal development. The maturation mediums and methods described herein can advantageously recapitulate heart development in vitro and enable organoids to acquire high levels of complexity and anatomical relevance by inducing progressive mitochondrial and metabolic maturation, electrophysiological maturation, increased morphological and cellular complexity, and recapitulating anterior-posterior heart tube patterning by endogenous retinoic acid signaling and self-organization

### A. Maturation Medium

A maturation medium is provided herein, which, for example, can be used for inducing development or maturing an early embryonic human heart organoid into a mature human heart organoid. A maturation medium includes a cell growth medium and a medium supplement.

Examples of suitable cell growth medium include, but are not limited to: Roswell Park Memorial Institute (RPMI) medium, for example, RPMI 1640 inclusive of its various formulations, such as with D-glucose, without D-glucose, with L-glutamine, without L-glutamine, with sodium bicarbonate, without sodium bicarbonate, HEPES modification, etc.; Dulbecco's Modified Eagle's Medium (DMEM) inclusive of its various formulations, such as high glucose, low glucose, with HEPES, etc.; a derivative of DMEM, such as Iscove's Modified Dulbecco's Medium (IMDM) or Advanced Dulbecco's Modified Eagle's Medium (ADMEM); or a combination thereof. These media are available from Thermo Fisher Scientific, Sigma-Aldrich, Millipore Sigma, and the like, and the same or the same trade name of a medium indicates the same medium composition irrespective of the manufacturers.

In any embodiment, the cell growth medium may be present in the maturation medium, based on total volume of the maturation medium, in an amount of greater than or equal to about 90 v/v %, greater than or equal to about 95 v/v %, greater than or equal to about 96 v/v %, greater than or equal to about 97 v/v %, greater than or equal to about 98 v/v %, or about 99 v/v %; or from about 90 v/v % to about 99 v/v %, about 95 v/v % to about 99 v/v %, about 96 v/v % to about 99 v/v % or about 97 v/v % to about 98 v/v %.

The medium supplement may include one or more fatty acids, triiodothyronine (T3) growth hormone, insulin, one or more antioxidant, a sugar, and carnitine. For example, the medium supplement may include one or more of the following: biotin, L-carnitine, corticosterone, ethanolamine, D(+)-galactose, glutathione (reduced), linoleic acid, linolenic acid, oleic acid, pipecolic acid, progesterone, putrescine, retinol acetate, sodium selenite, T3 growth hormone, DL-α-tocopherol (vitamin E), DL-α-tocopherol acetate, proteins, albumin bovine, catalase, insulin, superoxide dismutase, and transferrin. Exemplary suitable commercially available medium supplements include various B-27™ supplement formulations (available from Thermo Fisher Scientific), such as B-27™ Supplement (50×), serum free; B-27™ Supplement, minus insulin; B-27™ Plus Supplement (50×); B-27™ Supplement (50×), minus vitamin A; B-27™ Supplement (50×), minus antioxidants; and the like.

In any embodiment, the medium supplement may be present in the maturation medium, based on total volume of the maturation medium, in an amount of less than or equal to about 5 v/v %, less than or equal to about 4 v/v %, less than or equal to about 3 v/v %, greater than or equal to about 1 v/v %, or greater than or equal to about 2 v/v %; or from about 1 v/v % to about 5 v/v %, about 1 v/v % to about 4 v/v %, about 1 v/v % to about 3 v/v % or about 1 v/v % to about 2 v/v %.

Additionally or alternatively, the maturation medium may further include an antibiotic. Any suitable antibiotic used for cell cultures may be included. For example, the antibiotic may include amphotericin B, ampicillin, cephalothin, dihydrostreptomycin, gentamicin sulfate, penicillin streptomycin, kanamycin sulfate, lincomycin hydrochloride, neomycin sulfate, nystatin, paromomycin sulfate, penicillin-G, phenoxymethylpenicillinic acid, polymyxin B sulfate, spectinomycin, streptomycin, tetracycline hydrochloride, tylosin tartrate, or a combination thereof. It is contemplated herein that the antibiotic may be optional and is not required to be in the maturation medium. When present in the maturation medium, the antibiotic may be present in an amount, based on total volume of the maturation medium, of less than or equal to about 5 v/v %, less than or equal to about 4 v/v %, less than or equal to about 3 v/v %, greater than or equal to about 1 v/v %, or greater than or equal to about 2 v/v %; or from about 1 v/v % to about 5 v/v %, about 1 v/v % to about 4 v/v %, about 1 v/v % to about 3 v/v % or about 1 v/v % to about 2 v/v %.

In any embodiment, the maturation medium further includes one or more of the following additional components: one or more additional fatty acids, an additional carnitine or creatine, an additional T3 growth hormone, an additional sugar, and an additional antioxidant. As used herein, “additional component(s)” refer to a component that is present in an amount in addition to that component or class of component already present in the medium supplement. For example, the “additional T3 growth hormone” refers to T3 growth hormone present in the maturation medium in addition to the T3 growth hormone present in the medium supplement.

Suitable fatty acids include, but are not limited to, palmitic acid, oleic acid, linoleic acid, stearic acid, or a combination thereof. For example, a maturation medium may include palmitic acid, oleic acid, and linoleic acid. In any embodiment, oleic acid and linoleic acid may be present in the medium supplement as well as an additional amount present in the maturation medium. It is also contemplated herein that the one or more fatty acid may be admixed with bovine serum albumin (BSA) wherein the BSA is present in a negligible amount.

In any embodiment, one or more additional fatty acid, singularly or in combination, may be present in a maturation medium in an amount of greater than or equal to about 10 μM, greater than or equal to about 20 μM, greater than or equal to about 40 μM, greater than or equal to about 50 μM, less than or equal to about 100 μM, less than or equal to about 90 μM, less than or equal to about 80 μM, less than or equal to about 70 μM, or less than or equal to about 60 PM; or from about 10 μM to about 100 μM, about 10 μM to about 80 μM, about 10 μM to about 60 μM, about 10 μM to about 40 μM, about 20 μM to about 100 μM, about 20 μM to about 80 μM, or about 20 μM to about 60 μM. For example, a maturation medium may include about 20-60 μM of oleic acid in addition to oleic acid present in the medium supplement, and about 10-40 μM linoleic acid in addition to linoleic acid present in the medium supplement.

Additionally or alternatively, one or more fatty acid, singularly or in combination, may be present in a maturation medium in a total amount of greater than or equal to about 10 PM, greater than or equal to about 20 μM, greater than or equal to about 40 μM, greater than or equal to about 50 μM, less than or equal to about 100 μM, less than or equal to about 90 μM, less than or equal to about 80 μM, less than or equal to about 70 μM, or less than or equal to about 60 PM; or from about 10 μM to about 100 μM, about 10 μM to about 80 μM, about 10 μM to about 60 μM, about 20 μM to about 100 μM, about 20 μM to about 80 μM, or about 20 μM to about 60 μM. For example, a maturation medium may include a total amount of the following: about 20-80 μM palmitic acid, about 20-80 μM oleic acid, and about 10-60 μM linoleic acid.

Suitable carnitines include, but are not limited to L-carnitine, acetyl-L-carnitine, propionyl-L-carnitine, or a combination thereof. For example, a maturation medium may include additional L-carnitine.

In any embodiment, additional carnitine and/or creatine may each be present in a maturation medium in an amount of greater than or equal to about 60 PM, greater than or equal to about 80 μM, greater than or equal to about 100 μM, greater than or equal to about 120 μM, less than or equal to about 200 μM, less than or equal to about 180 μM, less than or equal to about 160 μM, or less than or equal to about 140 μM; or from about 60 μM to about 200 μM, about 60 μM to about 180 μM, about 60 μM to about 160 μM, about 80 μM to about 160 PM, about 100 μM to about 140 μM, or about 100 μM to about 130 μM. For example, a maturation medium may include about 60 μM to about 160 μM of carnitine in addition to carnitine present in the medium supplement.

Additionally or alternatively, carnitine and/or creatine may each be present in a maturation medium in a total amount of greater than or equal to about 60 PM, greater than or equal to about 80 μM, greater than or equal to about 100 μM, greater than or equal to about 120 μM, less than or equal to about 200 μM, less than or equal to about 180 μM, less than or equal to about 160 μM, or less than or equal to about 140 μM; or from about 60 μM to about 200 PM, about 60 μM to about 180 μM, about 60 μM to about 160 μM, about 80 μM to about 160 PM, or about 100 μM to about 140 μM. For example, a maturation medium may include about 60 μM to about 200 μM of carnitine and/or creatine in total.

In any embodiment, additional T3 growth hormone may be present in a maturation medium in an amount of greater than or equal to about 10 nM, greater than or equal to about 15 nM, greater than or equal to about 20 nM, greater than or equal to about 25 nM, less than or equal to about 50 nM, less than or equal to about 45 nM, less than or equal to about 40 nM, less than or equal to about 35 nM, or less than or equal to about 30 nM; or from about 10 nM to about 60 nM, about 10 nM to about 50 nM, about 10 nM to about 40 nM, about 20 nM to about 60 nM, about 20 nM to about 50 nM, or about 20 nM to about 40 nM. For example, a maturation medium may include about 10 nM to about 50 nM of T3 growth hormone in addition to T3 growth hormone present in the medium supplement.

Additionally or alternatively, T3 growth hormone may be present in a maturation medium in a total amount of greater than or equal to about 10 nM, greater than or equal to about 15 nM, greater than or equal to about 20 nM, greater than or equal to about 25 nM, less than or equal to about 50 nM, less than or equal to about 45 nM, less than or equal to about 40 nM, less than or equal to about 35 nM, or less than or equal to about 30 nM; or from about 10 nM to about 60 nM, about 10 nM to about 50 nM, about 10 nM to about 40 nM, about 20 nM to about 60 nM, about 20 nM to about 50 nM, or about 20 nM to about 40 nM. For example, a maturation medium may include about 10 nM to about 60 nM of T3 growth hormone in total.

Suitable sugars include, but are not limited to glucose, fructose, galactose, and combinations thereof. For example, a maturation medium may include glucose, which may be in addition to other sugar(s) present in the medium supplement.

In any embodiment, an additional sugar (e.g., glucose) may be present in a maturation medium in an amount of greater than or equal to about 1 mM, greater than or equal to about 2 mM, greater than or equal to about 3 mM, greater than or equal to about 4 mM, greater than or equal to about 5 mM, less than or equal to about 10 mM, less than or equal to about 9 mM, less than or equal to about 8 mM, less than or equal to about 7 mM, or less than or equal to about 6 nM; or from about 1 mM to about 10 mM, about 1 mM to about 8 mM, about 1 mM to about 6 mM, about 1 mM to about 5 mM, about 2 mM to about 8 mM or about 2 mM to about 6 mM. It is contemplated herein that the aforementioned amounts of an additional sugar may correspond to a total amount of said sugar (e.g., glucose) present in the maturation medium.

Suitable antioxidants include, but are not limited to ascorbic acid (vitamin C), glutathione, lipoic acid, uric acid, a carotene, a tocopherol (vitamin E), and ubiquinol, and combinations thereof. For example, a maturation medium may include ascorbic acid (vitamin C), which may be in addition to other antioxidant(s) present in the medium supplement.

In any embodiment, an antioxidant (e.g., ascorbic acid (vitamin C) may be present in a maturation medium in an amount of greater than or equal to about 0.1 mM, greater than or equal to about 0.2 mM, greater than or equal to about 0.3 mM, greater than or equal to about 0.4 mM, greater than or equal to about 0.5 mM, less than or equal to about 1 mM, less than or equal to about 0.9 mM, less than or equal to about 0.8 mM, less than or equal to about 0.7 mM, or less than or equal to about 0.6 nM; or from about 0.1 mM to about 1 mM, about 0.1 mM to about 0.8 mM, about 0.1 mM to about 0.6 mM, about 0.1 mM to about 0.5 mM, about 0.2 mM to about 0.8 mM or about 0.2 mM to about 0.6 mM. It is contemplated herein that the aforementioned amounts of an additional antioxidant may correspond to a total amount of said antioxidant (e.g ascorbic acid (vitamin C)) present in the maturation medium.

Additionally or alternatively, a maturation medium may further include a growth factor, such as IFG-1, IFG-2, or a combination thereof. In any embodiment, a growth factor may be present in a maturation medium in an amount of greater than or equal to about 5 ng/mL, greater than or equal to about 10 ng/mL, greater than or equal to about 20 ng/mL, greater than or equal to about 30 ng/mL, greater than or equal to about 40 ng/mL, greater than or equal to about 50 ng/mL, less than or equal to about 110 ng/mL, less than or equal to about 100 ng/mL, less than or equal to about 90 ng/mL, less than or equal to about 80 ng/mL, less than or equal to about 70 ng/mL, or less than or equal to about 60 ng/mL; or from about 5 ng/mL to about 110 ng/mL, about 10 ng/mL to about 100 ng/mL, about 20 ng/mL to about 90 ng/mL, about 30 ng/mL to about 70 ng/mL, or about 40 ng/mL to about 60 ng/mL.

In various aspects, a maturation medium may include a cell growth medium as described herein (e.g., RPMI 1640), a medium supplement as described herein (e.g., B-27™ supplement), one or more additional fatty acids as described herein (e.g., palmitic acid, oleic acid, linoleic acid), an additional carnitine as described herein (e.g., L-carnitine) or creatine, an additional T3 growth hormone and optionally, an antibiotic as described herein (e.g., penicillin streptomycin). For example, a mature medium may include about 97% RPMI 1640 medium; about 2% medium supplement (e.g., B-27™ supplement); about 1% penicillin streptomycin; about 52.5 μM palmitic acid in total; about 43.95 μM oleic acid in total; about 26 μM linoleic acid in total; about 132.2 μM L-carnitine in total, and about 33.01 nM T3 hormone in total.

In a further aspect, a maturation medium may include a cell growth medium as described herein (e.g., RPMI 1640), a medium supplement as described herein (e.g., B-27™ supplement), one or more additional fatty acids as described herein (e.g., palmitic acid, oleic acid, linoleic acid), an additional carnitine as described herein (e.g., L-carnitine) or creatine, an additional T3 growth hormone, an additional sugar as described herein (e.g., glucose), an additional antioxidant as described herein (e.g., ascorbic acid (vitamin C)), and optionally, an antibiotic as described herein (e.g., penicillin streptomycin). For example, a mature medium may include about 97% RPMI 1640 medium (no D-glucose); about 2% medium supplement (e.g., B-27™ supplement); about 1% penicillin streptomycin; about 52.5 μM palmitic acid in total; about 43.95 μM oleic acid in total; about 26 μM linoleic acid in total; about 132.2 μM L-carnitine in total, about 33.01 nM T3 hormone in total, about 0.4 mM ascorbic acid in total; and about 4 mM glucose in total.

In a further aspect, a maturation medium may include a cell growth medium as described herein (e.g., RPMI 1640), a medium supplement as described herein (e.g., B-27™ supplement), one or more additional fatty acids as described herein (e.g., palmitic acid, oleic acid, linoleic acid), an additional carnitine as described herein (e.g., L-carnitine) or creatine, an additional T3 growth hormone, an additional sugar as described herein (e.g., glucose), an additional antioxidant as described herein (e.g., ascorbic acid (vitamin C)), a growth factor as described herein (e.g., IGF-1), and optionally, an antibiotic as described herein (e.g., penicillin streptomycin). For example, a mature medium may include about 97% RPMI 1640 medium (no D-glucose); about 2% medium supplement (e.g., B-27™ supplement); about 1% penicillin streptomycin; about 52.5 μM palmitic acid in total; about 43.95 μM oleic acid in total; about 26 M linoleic acid in total; about 132.2 μM L-carnitine in total, about 33.01 nM T3 hormone in total, about 0.4 mM ascorbic acid in total; about 4 mM glucose in total, and about 50 ng/mL IGF-1.

In any embodiment, a maturation medium may not include exogenous retinoic acid and/or an extracellular matrix material, such as a hydrogel (e.g., Matrigel® Matrix). As used herein, “exogenous retinoic acid” refers to retinoic acid which is not naturally present in or produced by the human heart organoid.

### B. Maturation Methods

Methods for maturing an early embryonic human heart organoid into a mature human heart organoid are also provided herein. These methods can also be referred to as a developmental induction strategy. A method includes contacting an early embryonic human heart organoid with a maturation medium as described herein. As used herein an “early embryonic human heart organoid” refers to a three-dimensional body having an interior portion comprising myocardial tissue and outer surface comprising epicardial tissue and exhibits both first and second heart fields and cardiac chambers. The early embryonic heart organoid also may include at least one chamber or microchamber defined by the myocardial tissue, the at least one chamber or microchamber being lined with endocardial cells. Epicardial tissue (comprising epicardial cells) may be disposed on at least a portion of the surface. The early embryonic heart organoid may also include cardiac fibroblasts and endothelial vasculature and may beat. As used herein a “mature human heart organoid” encompasses a fetal-like human heart organoid and an adult human heart organoid, which can be achieved with longer culture periods. A “fetal-like human heart organoid” refers to an organoid with well-defined atrial and ventricular chambers. For example, a fetal-like human heart organoid may be considered comparable to a fetal human heart at about gestational day 45 to about gestational day 90. An “adult human heart organoid” refers to a heart organoid with well-defined atrial and ventricular chambers and metabolic and electrophysiological profiles characteristic of the adult heart (e.g., fatty acid metabolism, presence of atrial, ventricular and conductance action potentials).

In any embodiment, an early embryonic human heart organoid can be formed via methods known in the art. For example, an early embryonic human heart organoid can be formed from differentiation of human induced pluripotent stem cells (hiPSCs) as described by International Patent Publication No. WO 2021/257812, which is hereby incorporated by referenced in its entirety.

An early embryonic human heart organoid may be contacted with the maturation medium as described herein after start of the differentiation of the hiPSCs. Start of differentiation of the hiPCS may begin at day zero (0). Following day zero of the start of the differentiation of the hiPSCs, the early embryonic human heart organoid may be contacted with a maturation medium as described herein. For example, on any one of days 15 to day 25 following day zero of start of the differentiation of the hiPSCs, the early embryonic human heart organoid may be contacted with a maturation medium as described herein. In various aspects, the embryonic human heart organoid may be contacted with a maturation medium as described herein on day 20 following day zero of start of the differentiation of the hiPSCs.

In any embodiment, an early embryonic human heart organoid may be contacted with a maturation medium as described herein for a suitable amount of time to mature into a mature human heart organoid. For example, an early embryonic human heart organoid may be contacted with a maturation medium as described herein for greater than or equal to about 4 days, greater than or equal to about 6 days, greater than or equal to about 8 days, greater than or equal to about 9 days, less than or equal to about 16 days, less than or equal to about 14 days, less than or equal to about 12 days, less than or equal to about 11 days, or less than or equal to about 10 days; about 4 days to about 16 days, about 4 days to about 14 days, about 6 days to about 12 days, or about 8 days to about 10 days. In any embodiment, an early embryonic human heart organoid may be contacted with a maturation medium as described herein for about 10 days, for example, from about day 20 to about day 30 following day zero of start of the differentiation of the hiPSCs. At least a portion of the maturation medium contacting the early embryonic human heart organoid may be replaced with fresh maturation medium as needed, for example, every 24 hours to 72 hours, (e.g., every 24 hours, every 48 hours, every 72 hours). Fresh maturation medium may have the same or different composition than the maturation medium being replaced. It is also contemplated herein that a portion of the maturation medium being replaced remains in contact with the early embryonic human heart organoid. Alternatively, substantially all of the maturation medium contacting the early embryonic human heart organoid may be replaced with fresh maturation medium.

In various aspects, an early embryonic human heart organoid may be contacted, for example, any time from about day 20 to about day 30, with a maturation medium including a cell growth medium as described herein (e.g., RPMI 1640), a medium supplement as described herein (e.g., B-27™ supplement), one or more additional fatty acids as described herein (e.g., palmitic acid, oleic acid, linoleic acid), an additional carnitine as described herein (e.g., L-carnitine) or creatine, an additional T3 growth hormone and optionally, an antibiotic as described herein (e.g., penicillin streptomycin). For example, a mature medium may include about 97% RPMI 1640 medium; about 2% medium supplement (e.g., B-27™ supplement); about 1% penicillin streptomycin; about 52.5 μM palmitic acid in total; about 43.95 μM oleic acid in total; about 26 M linoleic acid in total; about 132.2 μM L-carnitine in total, and about 33.01 nM T3 hormone in total.

In a further aspect, an early embryonic human heart organoid may be contacted, for example, any time from about day 20 to about day 30, with a maturation medium including a cell growth medium as described herein (e.g., RPMI 1640), a medium supplement as described herein (e.g., B-27™ supplement), one or more additional fatty acids as described herein (e.g., palmitic acid, oleic acid, linoleic acid), an additional carnitine as described herein (e.g., L-carnitine) or creatine, an additional T3 growth hormone, an additional sugar as described herein (e.g., glucose), an additional antioxidant as described herein (e.g., ascorbic acid (vitamin C)), and optionally, an antibiotic as described herein (e.g., penicillin streptomycin). For example, a mature medium may include about 97% RPMI 1640 medium (no D-glucose); about 2% medium supplement (e.g., B-27™ supplement); about 1% penicillin streptomycin; about 52.5 μM palmitic acid in total; about 43.95 μM oleic acid in total; about 26 μM linoleic acid in total; about 132.2 μM L-carnitine in total, about 33.01 nM T3 hormone in total, about 0.4 mM ascorbic acid in total; and about 4 mM glucose in total.

In a further aspect, an early embryonic human heart organoid may be contacted, for example, any time from day 20 to about day 30, with a maturation medium including a cell growth medium as described herein (e.g., RPMI 1640), a medium supplement as described herein (e.g., B-27™ supplement), one or more additional fatty acids as described herein (e.g., palmitic acid, oleic acid, linoleic acid), an additional carnitine as described herein (e.g., L-carnitine) or creatine, an additional T3 growth hormone, an additional sugar as described herein (e.g., glucose), an additional antioxidant as described herein (e.g., ascorbic acid (vitamin C)), a growth factor as described herein (e.g., IGF-1), and optionally, an antibiotic as described herein (e.g., penicillin streptomycin). For example, a mature medium may include about 97% RPMI 1640 medium (no D-glucose); about 2% medium supplement (e.g., B-27™ supplement); about 1% penicillin streptomycin; about 52.5 μM palmitic acid in total; about 43.95 μM oleic acid in total; about 26 M linoleic acid in total; about 132.2 μM L-carnitine in total, about 33.01 nM T3 hormone in total, about 0.4 mM ascorbic acid in total; about 4 mM glucose in total, and about 50 ng/mL IGF-1.

It is also contemplated herein that an early embryonic human heart organoid may be contacted with more than one maturation medium as described herein, for example, during day 20 to day 30. In any embodiment, an early embryonic human heart organoid may be contacted, for example, from day 20 to day 26, with a maturation medium (a first maturation medium) including a cell growth medium as described herein (e.g., RPMI 1640), a medium supplement as described herein (e.g., B-27™ supplement), one or more additional fatty acids as described herein (e.g., palmitic acid, oleic acid, linoleic acid), an additional carnitine as described herein (e.g., L-carnitine) or creatine, an additional T3 growth hormone, and further including an additional antioxidant as described herein, such as ascorbic acid, an additional sugar as described herein, such as glucose, a growth factor as described herein, such as IGF-1, and optionally, an antibiotic as described herein (e.g., penicillin streptomycin). Additionally, the embryonic human heart organoid may be contacted, for example, from day 26 to day 30, with a maturation medium (a second maturation medium) including a cell growth medium as described herein (e.g., RPMI 1640), a medium supplement as described herein (e.g., B-27™ supplement), one or more additional fatty acids as described herein (e.g., palmitic acid, oleic acid, linoleic acid), an additional carnitine as described herein (e.g., L-carnitine) or creatine, an additional T3 growth hormone, and further including an additional antioxidant as described herein, such as ascorbic acid, an additional sugar as described herein, such as glucose, wherein the maturation medium does not contain a growth factor as described herein, such as IGF-1. In any embodiment, a portion of the maturation medium from day 20 to day 26 (first maturation medium) may contact the early embryonic human heart organoid form day 26 onward, for example, from day 26 to day 30. In other words, on day 26 when the first maturation medium is changed, instead of removing all of the first maturation medium used from day 20 to day 26, only a portion that first maturation medium may be removed and replaced with a second maturation medium.

In any embodiment, exogenous retinoic acid and/or an extracellular matrix material may not be added during the methods described herein.

A mature human heart organoid produced by the methods described herein is also provided.

It was discovered that that methods described herein utilizing the maturation mediums described herein can advantageously produce human heart organoids with several unique and crucial characteristics representative of an early fetal human heart, including the presence of anteroposterior patterning with a retinoic acid gradient at the posterior pole and polar separation of atrioventricular chambers with an epicardial layer at the anterior end. Additionally, the disclosed methods may also result in human heart organoids with valvular cells, conductance cells, proepicardial cells and more, as well as large hollow chambers, functional electrophysiology, and increased mitochondrial density and metabolic transcriptional profiles similar to that of gestational human hearts. For example, single-cell gene expression for key genes relating to multiple cell clusters revealed that human heart organoids produced by contacting an early embryonic human heart organoid with two different maturation mediums from day 20 to day 30 as described above, can yield the highest similarity to in vivo 6.5 post-conception week (GD45) developing human hearts⁴¹. Further, the strategies described herein led to expansion and reduction of certain cardiac cell type populations, such as atrial and ventricular cardiomyocytes, and mesenchymal cell types (stromal cells) in what is believed to be a process of fine tuning and remodeling. Interestingly, the appearance of valvular and conductance cell types were observed for the human heart organoids produced herein.

The mature human heart organoids produced herein respond dramatically to developmental maturation stimuli and metabolically maturate and possess increased mitochondrial growth, density respiration rates and gene expression. These dramatic responses compared to traditional methods may be the result of synergy between multiple cardiac cell subtypes, such as epicardial cells and cardiac fibroblasts, which have been shown to stimulate cardiomyocyte growth and function^{121,122}.

Furthermore, proper and gradual electrophysiological maturation throughout the cardiac syncytium, including the complex interplay between various ion channels and their subtypes as well as depolarization through t-tubules, comprises critical aspects of cardiac development and functionality^{103-106,126,127,145}. It was discovered that human heart organoids produced via the methods described herein, for example, by contacting an early embryonic human heart organoid with two different maturation mediums from day 20 to day 30 as described above, develop distinct calcium transients with increasing physiological mimicry due to their increased amplitude and decreased frequency. Additionally, these organoids can develop higher levels of t-tubules, inward-rectifying potassium ion channels, and hERG channels compared to other maturation strategies. In fact, many efforts towards in vitro cardiomyocyte maturation have struggled or failed to elicit the presence of t-tubules^{146,147} and inward-rectifying potassium ion channels remain critical to establishing low resting membrane potential^{148,149}. Moreover, cardiac hERG channels represent a paramount channel of importance for pharmacological screening due to its high arrhythmogenic potential if interfered with^{116,117}. Nonetheless, the summation of multiple ion transients results in the cardiac action potential which is the ultimate driving force for human heart contraction and functionality. Furthermore, these produced organoids possess ventricular-, atrial-, and nodal-like action potentials, opening the door for electrophysiological applications in drug screening.

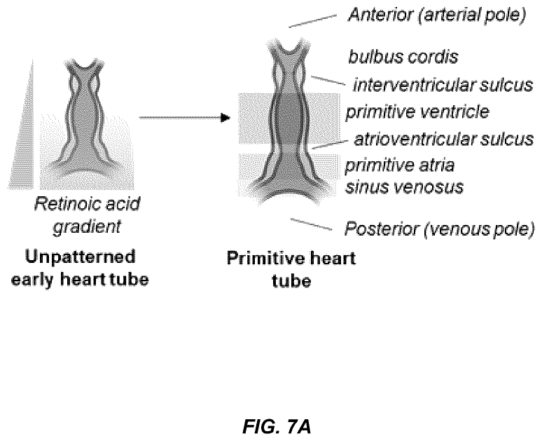

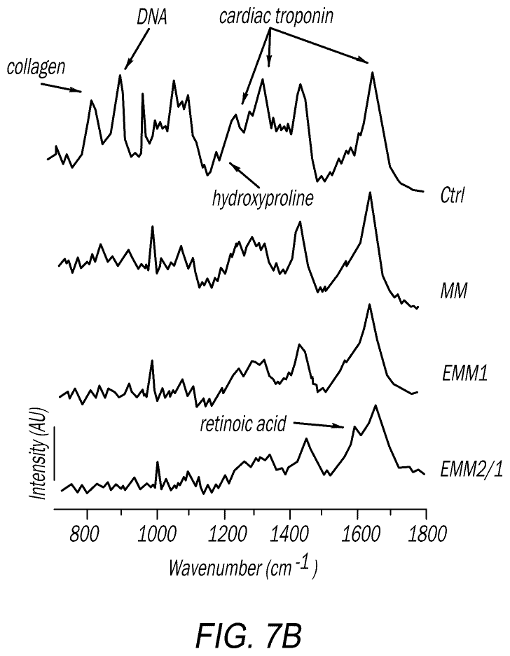

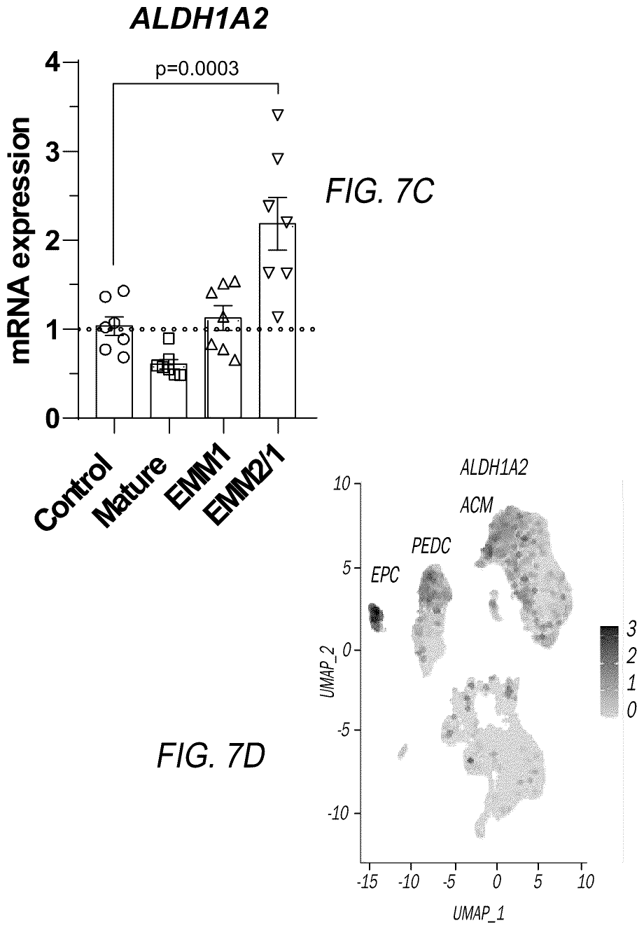

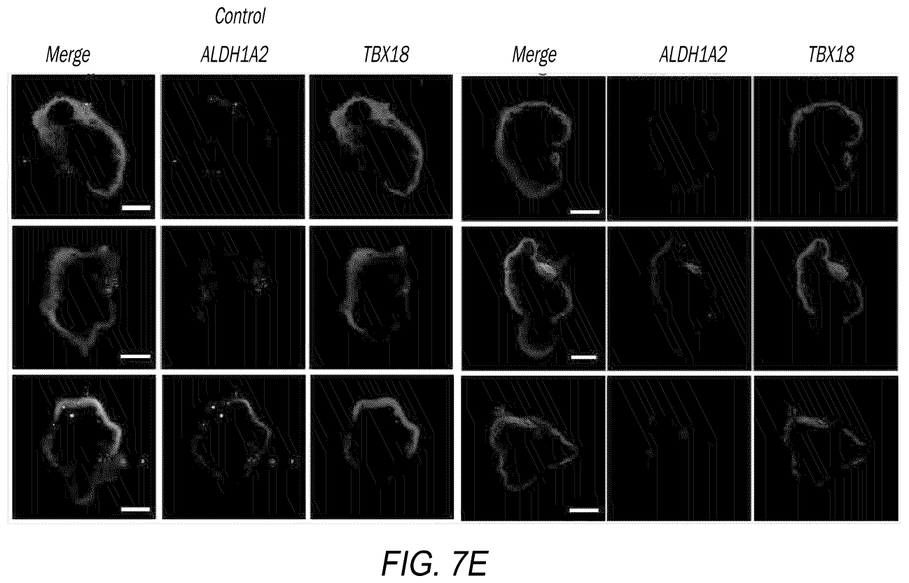

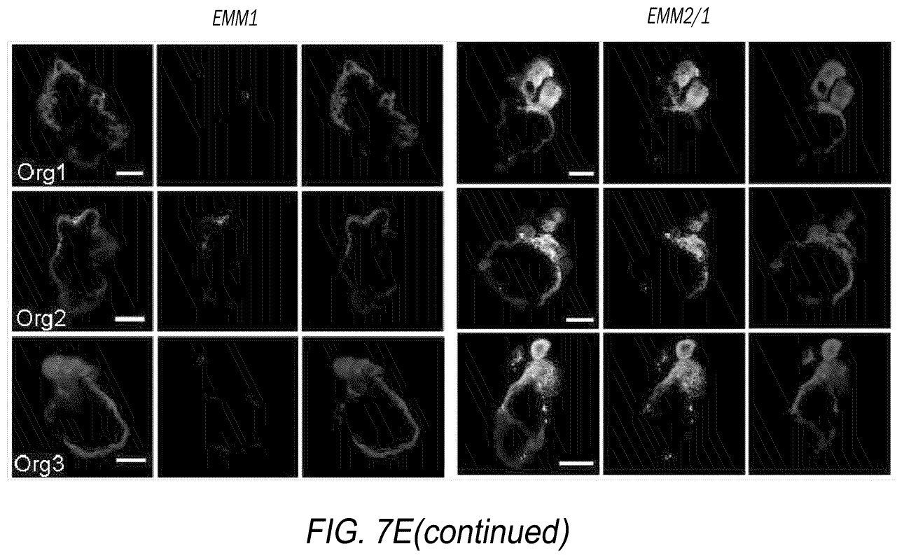

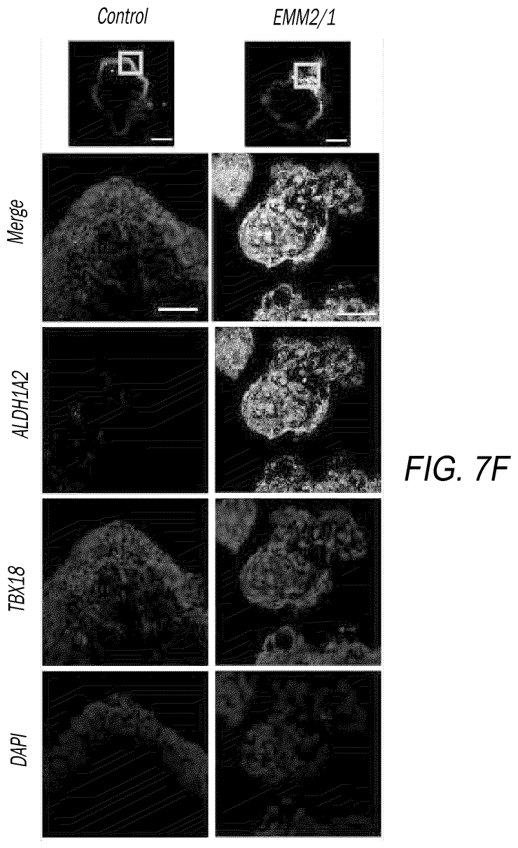

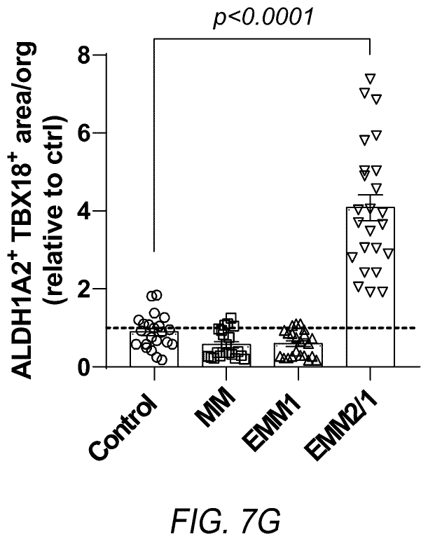

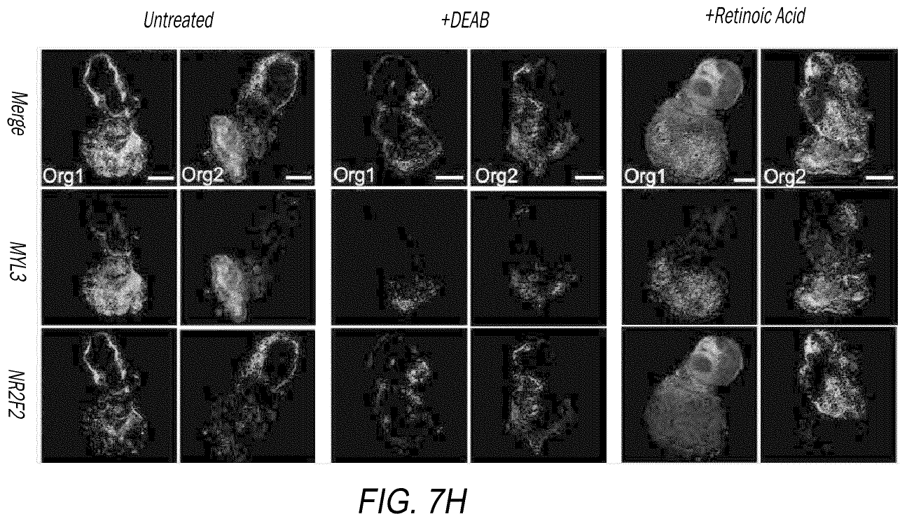

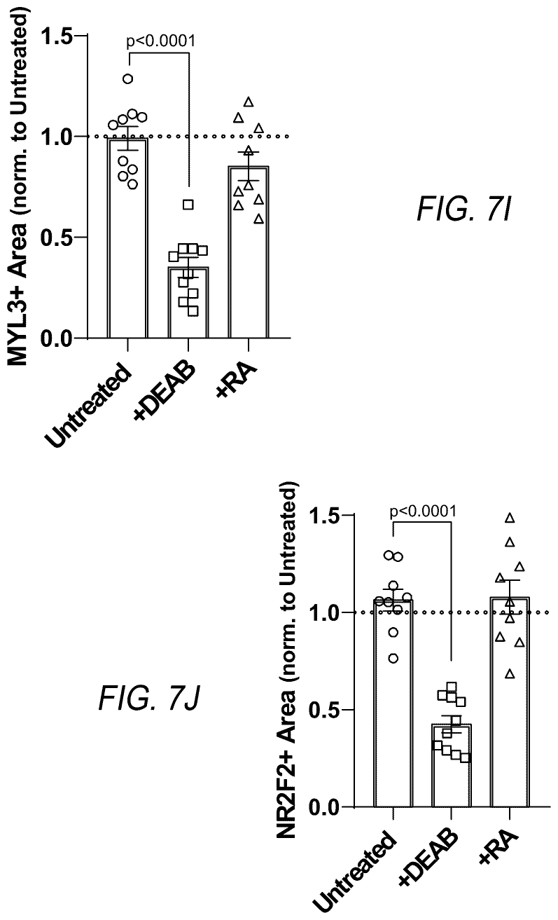

The embryonic heart begins as an unpatterned heart tube and undergoes cellular and structural changes through morphogenetic signaling events to pattern along the anterior-posterior axis, loop and eventually form the 4-chambered heart^{150,151}. It was surprisingly found that human heart organoids produced via the maturation methods described herein, for example, when an early embryonic human heart organoid is contacted with two different maturation mediums from day 20 to day 30 as described above, formed a two-chambered structure with cardiomyocytes forming one chamber with atrial identity and another with ventricular fate. Dense epicardial layering at the atrial chamber identified the proepicardial organ as the posterior pole of the heart tube¹⁵² and revealed that these organoids were spontaneously patterning along the aforementioned anterior-posterior axis. It was further found that this self-organization and patterning in these organoids was driven by an endogenous retinoic acid signaling gradient. ALDH1A2, an enzyme required for retinoic acid synthesis, was observed to be spatially restricted to the posterior end of the organoid, and co-localized with TBX18, an epicardial transcription factor confirming that the proepicardial organ was functional. Thus, in various aspects, the methods described herein may produce a mature human heart organoid including one or more of the following characteristics: (i) endogenous retinoic acid; (ii) at least cardiac two chamber (e.g., an atrial chamber and a ventricular chamber); (iii) a posterior proepicardial pole (see FIG. 7A); and (iv) anterior-posterior heart tube patterning (e.g., anterior-posterior patterning of ventricular (anterior pole) and atrial (posterior) chambers) (see FIG. 7A). Additionally, the methods described herein may produce a human heart organoid with valvular cells, conductance cells, proepicardial cells and more, as well as large hollow chambers, functional electrophysiology, and increased mitochondrial density and metabolic transcriptional profiles similar to that of gestational human hearts. As used herein, “endogenous retinoic acid” refers to retinoic acid that is naturally found in or produced by the mature human heart organoid, for example, by day 30 of the method. The endogenous retinoic acid may be present as a gradient in the mature human heart organoid. As discussed above, retinoic acid is a powerful morphogen involved in cardiac development and provides instructions to the heart for cellular development and patterning. The retinoic acid gradient may originate and/or be localized at a posterior pole (proepicardial/atrial pole) of the obtained human heart organoid. Additionally, the mature human heart organoid may be capable of beating, for example, 60 to 80 beats per minute. With such characteristics, the mature human heart organoid described herein may be considered comparable to a fetal human heart at about gestational day 45 to about gestational day 90.

Taken together, it is believed that the human heart organoids produced by the methods described herein can recapitulate events that take place during in utero gestation where the proepicardial organ surrounds the posterior pole of the patterned heart tube where posterior atrial cardiomyocytes and proepicardial cells produce retinoic acid to form a signaling gradient that further instructs the remainder of the heart tube with patterning and specification information^{41,56,131,134,152}.

### EXAMPLES

### Example 1

### Methods and Procedures

Stem cell culture. The following human induced pluripotent stem cell (hiPSC) lines and human embryonic stem cell (ESC) lines were used for this study: iPSC-L1 (iPSC) (Sex: Male), ATCC-BYS0111 (iPSC) (Sex: Male) (Alias: ATCC), H9 (ESC) (Sex: Female) (WiCell, WA09). Pluripotency and genomic stability were tested for all hiPSC lines used. hiPSCs were cultured in Essential 8 Flex medium with 1% penicillin streptomycin (Gibco) in 6-well plates on growth factor reduced Matrigel (Corning) inside an incubator at 37° C. and 5% CO₂. hiPSCs were passaged using ReLeSR passaging reagent (STEMCELL Technologies) upon reaching 60-80% confluency. Unless otherwise specified, all data in the below results are from the iPSC-L1 line.

Self-assembling human heart organoid differentiation. Step-by-step, detailed protocols which describe the generation and differentiation of the human heart organoids are provided¹⁴. In brief, hiPSCs were grown to 60% confluency on 6-well plates and dissociated using Accutase (Innovative Cell Technologies) to obtain a single-cell solution. hiPSCs were collected and centrifuged at 300 g for 5 minutes and resuspended in Essential 8 Flex medium containing 2 μM ROCK inhibitor (Thiazovivin) (Millipore Sigma). hiPSCs were counted using a Moxi cell counter (Orflo Technologies) and were seeded at a concentration of 10,000 cells per well in round bottom 96 well ultra-low attachment plates (Costar) on day −2 in a volume of 100 μL. The plate was then centrifuged at 100 g for 3 minutes and subsequently placed inside a 37° C. and 5% CO₂ incubator. After 24 hours (day −1), 50 μL was removed from each well and 200 μL of fresh Essential 8 Flex Medium was added to each well to obtain a final volume of 250 μL per well. The plate was then placed inside a 37° C. and 5% CO₂ incubator. After 24 hours (day 0), 166 μL of medium was removed from each well. Then, 166 μL of RPMI with B27 supplement without insulin (Gibco) supplemented with 1% penicillin streptomycin (Gibco) (hereafter termed “RPMI/B27 minus insulin”) containing CHIR99021, BMP4, and Activin A was added to each well to obtain final concentrations of 4 μM CHIR99021, 36 μM (1.25 ng/mL) BMP4, and 8 μM (1.00 ng/mL) Activin A. The plate was subsequently placed inside a 37° C. and 5% CO₂ incubator. After 24 hours (day 1), 166 μL of medium was removed from each well and replaced with 166 μL of fresh RPMI/B27 minus insulin. On day 2, 166 μL of medium was removed from each well and 166 μL of 5 RPMI/B27 minus insulin with Wnt-C59 (Selleck) was added to obtain a final concentration of 2 M Wnt-C59 inside each well. The plate was then incubated for 48 hours. On day 4, 166 μL was removed and replaced with fresh RPMI/B27 minus insulin and incubated for 48 hours. On day 6, 166 μL was removed and replaced with 166 μL RPMI with B27 supplement (with insulin) and 1% penicillin streptomycin (hereafter termed “RPMI/B27”). The plate was incubated for 24 hours. On day 7, 166 μL of media was removed from each well and 166 μL of RPMI/B27 containing CHIR99021 was added to obtain a final concentration of 2 μM CHIR99021 per well. The plate was incubated for 1 hour. After 1 hour, 166 μL of medium was removed from each well and 166 L of fresh RPMI/B27 was added to each well. The plate was incubated for 48 hours. From days 9 to 19, every 48 hours, media changes were performed by removing 166 μL of media from each well and adding 166 L of fresh RPMI/B27.

Developmental induction conditions. Organoids were generated and differentiated according to the protocol outlined previously. Beginning on day 20, organoids were subjected to various maturation medium conditions. The control strategy was a continuation of culture within RPMI/B27 from day 20 to day 30, performing standard media changes every 48 hours.

The maturation medium (MM) strategy was employed from day 20 to day 30, performing media changes every 48 hours using MM media, consisting of stock RPMI/B27 (with insulin) with 52.5 μM palmitate-BSA, 40.5 μM oleate-BSA (Sigma), 22.5 μM lineolate-BSA (Sigma), 120 μM L-Carnitine (Sigma) and 30 nM T3 hormone (Sigma). The formulation details for MM media are provided below in Table 1.

TABLE 1
MM Media

| | |
|---|---|
| | Amount in total |
| Ingredient/Source | formulation |

| RPMI 1640 media (Thermo Fisher Scientific | 97% | |
|---|---|---|
| catalog #11875119) | | |
| B-27 ™ Supplement (50X), serum free with | 2% | |
| insulin (Thermo Fisher Scientific catalog | | |
| #17504044) | | |
| penicillin streptomycin (Thermo Fisher | 1% | |
| Scientific catalog #15140122) | | |
| palmitate-BSA (Cayman Chemical) | 52.5 | μM |
| Oleic acid (Sigma catalog #O1383) | 43.95 | μM |
| Linoleic acid (Sigma catalog #L1012) | 26.00 | μM |
| L-carnitine (Sigma catalog #8400920025) | 132.2 | μM |
| T3 hormone (Sigma catalog #T2877) | 33.01 | nM |

The enhanced maturation medium 1 (EMM1) strategy was employed from day 20 to day 30, performing media changes every 48 hours using EMM1 media, consisting of stock RPMI 1640 Medium, no glucose (Gibco) supplemented with B27 (with insulin), 1% penicillin streptomycin (Gibco), 52.5 μM palmitate-BSA, 40.5 μM oleate-BSA (Sigma), 22.5 μM lineolate-BSA (Sigma), 120 μM L-Carnitine (Sigma), 30 nM T3 hormone (Sigma), 0.4 mM ascorbic acid (Thermo Fisher Scientific) and 4 mM Glucose (Gibco). The formulation details for EMM1 media are provided below in Table 2.

TABLE 2
EMM1 Media

| | |
|---|---|
| | Amount in total |
| Ingredient/Source | formulation |

| RPMI 1640 media, No D-Glucose (Thermo | 97% | |
|---|---|---|
| Fisher Scientific catalog #11879020) | | |
| B-27 ™ Supplement (50X), serum free with | 2% | |
| insulin (Thermo Fisher Scientific catalog | | |
| #17504044) | | |
| penicillin streptomycin (Thermo Fisher | 1% | |
| Scientific catalog #5140122) | | |
| palmitate-BSA (Cayman Chemical) | 52.5 | μM |
| Oleic acid (Sigma catalog #O1383) | 43.95 | μM |
| Linoleic acid (Sigma catalog #L1012) | 26.00 | μM |
| L-carnitine (Sigma catalog #8400920025) | 132.2 | μM |
| T3 hormone (Sigma catalog #T2877) | 33.01 | nM |
| ascorbic acid (Thermo Fisher Scientific | 0.4 | mM |
| catalog #A25215G) | | |
| glucose (Thermo Fisher Scientific catalog | 4 | mM |
| #A2494001) | | |

The enhanced maturation medium 2/1 (EMM2/1) strategy was employed from day 20 to day 30, performing media changes every 48 hours, utilizing a combination of two medias. From day 20 to day 26, EMM2 media was utilized which consists of stock RPMI 1640 Medium, no glucose (Gibco) supplemented with B27 (with insulin), 1% penicillin streptomycin (Gibco), 52.5 M palmitate-BSA, 40.5 μM oleate-BSA (Sigma), 22.5 μM lineolate-BSA (Sigma), 120 μM L-Carnitine (Sigma), 30 nM T3 hormone (Sigma), 0.4 mM ascorbic acid (Thermo Fisher Scientific), 4 mM Glucose (Gibco) and 50 ng/mL IGF-1 (Sigma). Continuing the EMM2/1 strategy, from day 26 to day 30, EMM1 media was utilized. The formulation details for EMM2 media are provided below in Table 3.

TABLE 3
EMM2 Media

| | |
|---|---|
| | Amount in total |
| Ingredient/Source | formulation |

| RPMI 1640 media, No D-Glucose (Thermo | 97% | |
|---|---|---|
| Fisher Scientific catalog #11879020) | | |
| B-27 ™ Supplement (50X), serum free with | 2% | |
| insulin (Thermo Fischer Scientific catalog | | |
| #17504044) | | |
| penicillin streptomycin (Thermo Fisher | 1% | |
| Scientific catalog #5140122) | | |
| palmitate-BSA (Cayman Chemical) | 52.5 | μM |
| Oleic acid (Sigma catalog #O1383) | 43.95 | μM |
| Linoleic acid (Sigma catalog #L1012) | 26.00 | μM |
| L-carnitine (Sigma catalog #8400920025) | 132.2 | μM |
| T3 hormone (Sigma catalog #T2877) | 33.01 | nM |
| ascorbic acid (Thermo Fisher Scientific | 0.4 | mM |
| product #A25215G) | | |
| glucose (Thermo Fisher Scientific catalog | 4 | mM |
| #A2494001) | | |
| IGF-1 (Sigma catalog #I1271) | 50 | ng/mL |

Organoids were collected on day 30 for analysis.

Immunofluorescence. Human heart organoids were transferred from the round bottom ultra-low attachment 96 well plate to 1.5 mL microcentrifuge tubes (Eppendorf) using a cut 200 μL pipette tip (to increase tip bore diameter as to not disturb the organoid). Organoids were fixed in 4% paraformaldehyde (VWR) in PBS for 30 minutes. Following, organoids were washed using PBS-Glycine (1.5 g/L) three times for 5 minutes each. Organoids were then blocked and permeabilized using a solution containing 10% Donkey Normal Serum (Sigma), 0.5% Triton X-100 (Sigma), and 0.5% BSA (Thermo Fisher Scientific) in PBS on a thermal mixer at 300 rpm at 4° C. overnight. Organoids were then washed 3 times using PBS and incubated with primary antibodies (Table 4) within a solution containing 1% Donkey Normal Serum, 0.5% Triton X-100, and 0.5% BSA in PBS (hereafter termed “Antibody Solution”) on a thermal mixer at 300 rpm at 4° C. for 24 hours.

TABLE 4
Antibodies used for immunofluorescence.

| | Antibody | Host | | Catalogue | |
|---|---|---|---|---|---|
| | Name | Species | Dilution | Number | Vendor |
| Primary | cTnT | Mouse/ | 1:200 | ab8295 | Abcam |
| | (TNNT2) | Rabbit | | | |
| | WT1 | Rabbit | 1:200 | ab89901 | Abcam |
| | PECAM1 | Mouse | 1:50 | ab28364 | Abcam |
| | MLC2V | Rabbit | 1:200 | ab79935 | Abcam |
| | (MYL2) | | | | |
| | MLC2A | Mouse | 1:200 | 311-011 | Synaptic |
| | (MYL7) | | | | Systems |
| | KCNJ2 | Rabbit | 1:500 | HPA029109 | Sigma |
| Secondary | Alexa | Donkey | 1:200 | A-21202 | Thermo |
| | Fluor 488 | anti-mouse | | | Fisher |
| | | | | | Scientific |
| | Alexa | Donkey | 1:200 | A-21206 | Thermo |
| | Fluor 488 | anti-rabbit | | | Fisher |
| | | | | | Scientific |
| | Alexa | Donkey | 1:200 | A-21203 | Thermo |
| | Fluor 594 | anti-mouse | | | Fisher |
| | | | | | Scientific |
| | Alexa | Donkey | 1:200 | A-21207 | Thermo |
| | Fluor 594 | anti-rabbit | | | Fisher |
| | | | | | Scientific |

Following, organoids were washed 3 times for 5 minutes each using PBS. Organoids were then incubated with secondary antibodies (Table 4) in Antibody Solution on a thermal mixer at 300 rpm at 4° C. for 24 hours in the dark. Subsequently, organoids were washed 3 times for 5 minutes each using PBS and mounted on glass microscope slides (Fisher Scientific). 90 μm Polybead Microspheres (Polyscience, Inc.) were placed between the slide and a No. 1.5 coverslip (VWR) to provide support pillars such that the organoids could retain three dimensionality. Organoids were transferred to the glass microscope slides using a cut 200 uL pipette tip and mounted using a clearing solution described previously¹⁵³. T-tubule staining was performed using FITC-conjugated Wheat Germ Agglutinin (WGA) lectins (Sigma).

Confocal Microscopy and Image Analysis. Immunofluorescence images were acquired using a confocal laser scanning microscope (Nikon Instruments A1 Confocal Laser Microscope). Images were analyzed using Fiji. When comparing images across or between conditions, for each channel of an image being measured, pixel intensity values of images were equalized to that of the Control or EMM2/1 condition, where appropriate. To measure organoid diameter and area, the straight line and freehand tools were used, respectively. To measure mitochondrial (MitoTracker) area, Cav-3+ area, KCNJ2+ puncta, MYL2+ area, MYL7+ area, PECAM1+ area, and ALDH1A2+ TBX18+ area, the auto threshold function was utilized. To measure TNNT2+ and WT1+ chamber size, the oval selection tool was utilized and the wall of the organoid was used as the boundary region of the respective area to be drawn. For area measurements that utilized the whole organoid (low magnification images), datapoints were normalized to organoid area. To measure FlipGFP fluorescence intensity, the mean gray value was calculated. To measure Pearson's coefficient, the JaCOP colocalization plugin was used (Bolte, S., & Cordelieres, F. P. (2006). A guided tour into subcellular colocalization analysis in light microscopy. Journal of Microscopy, 224(3), 213-232. doi:10.1111/j.1365-2818.2006.01706.x). Thresholds were generated for the equalized image intensity values. A spatial resolution of 1.243 micrometers per pixel was utilized.

Heart organoid dissociation. Organoids were collected on day 30 from each maturation strategy (Control, MM, EMM1, EMM2/1). Organoids were individually placed into separate 1.5 mL microcentrifuge tubes (Eppendorf), dissociated and pooled. Organoids were dissociated into a single-celled suspension using a modified protocol of the STEMdiff Cardiomyocyte Dissociation Kit (STEMCELL Technologies). Upon being transferred to a microcentrifuge tube, organoids were washed with PBS, submerged in 200 L of warm dissociation media (37° C.), and placed on a thermal mixer at 37° C. and 300 rpm for 5 minutes. Then, the supernatant was collected and transferred to a 15 mL falcon tube (Corning) containing 5 mL of respective media (Control, MM, EMM1, etc.) containing 2% BSA (Thermo Fisher Scientific). An additional 200 μL of warm dissociation media (37° C.) was then added back to the organoid on a thermal mixer (37° C.). The organoid dissociation media solution was then pipetted up and down gently 3-5 times. The organoid was allowed to sit on the thermal mixer for an additional 5 minutes. If the organoid remained visible, the process was repeated. Once the organoid was no longer visible, the microcentrifuge tube solution was pipetted up and down gently 3-5 times and its entire contents were transferred to the 15 mL falcon tube containing the respective media+2% BSA and cells. These tubes were then centrifuged at 300 g for 5 minutes. The supernatant was aspirated, and the cell pellets were resuspended in respective media+2% BSA. Using a hemocytometer, viability, cell counts, and aggregate percentage were acquired.

Single cell RNA sequencing. Libraries were prepared using the 10× Chromium Next GEM Single Cell 3′ Kit, v3.1 and associated components. Completed libraries were QC'd and quantified using a combination of Qubit dsDNA HS, Agilent 4200 TapeStation HS DNA1000 and Invitrogen Collibri Library Quantification qPCR assays. The libraries were pooled in equimolar proportions and the pool quantified again using the Invitrogen Collibri qPCR assay. The pool was loaded onto two lanes of an Illumina NovaSeq 6000 SP flow cell (v1.5) and sequencing was performed in a custom paired end format, 28 cycles for read 1, 2 10 cycle index reads and 90 cycles for read 2. A v1.5, 100 cycle NovaSeq reagent cartridge was used for sequencing. The 28 bp read 1 includes the 10× cell barcodes and UMIs, read 2 is the cDNA read. Output of Real Time Analysis (RTA) was demultiplexed and converted to FastQ format with Illumina Bcl2fastq v2.20.0. After demultiplexing, reads from each of the sample libraries were further processed using 10× Genomics cellranger count (v6.1.2). Analysis of files was performed using 10× Genomics Loupe Browser v6.3.0 using k-means clustering of 8 clusters and UMAP visualization. Enrichr¹⁵⁴⁻¹⁵⁶ was used to assess gene ontologies. Pathview Web was used to generate biological pathway graphs^{101,102}. Cell-cell communication analysis was performed using Liana¹⁵⁷ and Celltalker¹⁵⁸ (https://github.com/arc85/celltalker). To accomplish this task, the counts and clusters data from Loupe Browser were imported into Seurat¹⁵⁹. Both programs perform differential expression between clusters using standard Seurat findmarkers differential expression function and then rank pairs of ligands and receptors by significant p-value and log 2fold change. To identify pairs of ligands and receptors pairs, Liana uses OmniPath database¹⁶⁰, and Celltalker uses Ramilowski-pairs database for ligands-receptors interactions¹⁶¹.

Transmission Electron Microscopy (TEM). Human heart organoids were fixed on Day 15 and Day 30 in 2.5% glutaraldehyde (Electron Microscopy Solutions) in PBS for 45 minutes, washed three times in PBS for 5 minutes each, then stored at 4° C. Samples were then washed with 100 mM phosphate buffer and postfixed with 1% osmium tetroxide in 100 mM phosphate buffer, dehydrated in a gradient series of acetone and infiltrated and embedded in Spurr (Electron Microscopy Sciences). 70 nm thin sections were obtained with a Power Tome Ultramicrotome (RMC, Boeckeler Instruments. Tucson, AZ) and post stained with uranyl acetate and lead citrate. A JEOL 1400Flash Transmission Electron Microscope (Japan Electron Optics Laboratory, Japan) was used to acquire images at an accelerating voltage of 100 k.

Calcium imaging. Calcium transient activity within the human heart organoids was assessed using Fluo-4 AM (Thermo Fisher Scientific). Fluo-4 AM was solubilized in DMSO per the manufacturer's instructions. A 1.5 μM solution of Fluo-4 was prepared in respective medium (Control, MM, EMM1, etc.). Organoids were washed twice using RPMI 1640 basal medium, then Fluo-4 AM was added at a final concentration of 1 μM and incubated for 30 minutes at 37° C. and 5% CO₂. Organoids were then washed twice using their respective medium (Control, MM, EMM1, etc.) and transferred to a chambered coverglass slide (Cellvis) using a cut 200 μL pipette tip. Videos were acquired using a Cellvivo microscope (Olympus) at 100 frames per second over 10 total seconds as an image stack. Samples were excited at 494 nm excitation and 506 nm emission was collected. Data was processed using Fiji and Microsoft Excel. Baseline F₀ of fluorescence intensity F was calculated using the average of the lowest 50 intensity values in the acquired dataset. Fluorescence change ΔF/F₀ was calculated using the equation:

$$\frac{Δ F}{{F}_{0}} = \frac{( F - {F}_{0} )}{{F}_{0}}$$

Voltage imaging. Voltage activity within human heart organoids was assessed using di-8-ANEPPS (Thermo Fisher Scientific). Di-8-ANEPPS was solubilized in DMSO per the manufacturer's instructions. A 15 μM solution of di-8-ANEPPS was prepared in respective medium (Control, MM, EMM1, etc.). Organoids were washed twice using RPMI 1640 basal medium, then di-8-ANEPPS was added at a final concentration of 10 μM and incubated for 30 minutes at 37° C. and 5% CO₂. Organoids were then washed twice using their respective medium (Control, MM, EMM1, etc.) and transferred to a chambered coverglass slide (Cellvis) using a cut 200 μL pipette tip. Videos were acquired using a Cellvivo microscope (Olympus) at 100 frames per second over 10 total seconds as an image stack at either 20× magnification (ondansetron) or 100× magnification (maturation electrophysiology). Samples were excited at 465 nm excitation and 630 nm emission was collected. Data was processed using Fiji and Microsoft Excel. Baseline F0 of fluorescence intensity F was calculated using the average of the lowest 50 intensity values in the acquired dataset. Fluorescence change ΔF/F0 was calculated using the same method as with Calcium imaging presented above. APD30 and APD90 were measured from the midpoint of the upstroke until 30% or 90% repolarization, respectively¹⁶².

Real time RT-PCR. Organoids were collected at day 20, 21, 23, 25, and 30 and stored in RNAprotect (Qiagen) at −20° C. RNA was extracted using the Qiagen RNEasy Mini Kit according largely to the manufacturer's instructions. Organoids were lysed using the Bead Mill 4 Homogenizer (Fisher Scientific) at speed 2 for 30 seconds. RNA concentration was measured using a NanoDrop One (Thermo Fisher Scientific). A minimum threshold of 10 ng/L was required to proceed with reverse transcription. cDNA was generated using the Quantitect Reverse Transcription Kit (Qiagen) and stored at −20° C. Primers for real time qPCR were designed using the Primer Quest tool (Integrated DNA Technologies). SYBR Green (Thermo Fisher Scientific) was used as the DNA intercalating dye and the amplifier for the reaction vessel. Real time qPCR was performed using the QuantStudio 5 Real-Time PCR system (Applied Biosystems) using a total reaction volume of 20 μL. Gene expression levels were normalized to HPRT1 expression in each independent sample. Log base 2 fold change values were obtained using the double delta CT method. At least 4 independent samples were run for each gene expression assay at each timepoint per condition. mRNA expression figures are displayed as log base 2 fold change relative to Control.

Mitochondrial imaging. Intracellular mitochondrial presence within human heart organoids was visualized using Mitotracker Deep Red FM (Thermo Fisher Scientific). Mitotracker was prepared according to the manufacturer's instructions. A 150 nM solution of Mitotracker was prepared in respective medium (Control, MM, EMM1, etc.). Additionally, NucBlue (Thermo Fisher Scientific) was used to visualize cell nuclei. NucBlue was prepared by adding 2 drops per milliliter of 150 nM Mitotracker solution (described above). Organoids were washed twice using 166 μL of RPMI 1640 basal medium, then 166 μL of Mitotracker was added to achieve a final concentration of 100 nM and incubated for 30 minutes at 37° C. and 5% CO₂. Organoids were incubated for 30 minutes at 37° C. and 5% CO₂. Organoids were then washed twice using their respective medium (Control, MM, EMM1, etc.) and transferred to a chambered coverglass slide (Cellvis) using a cut 200 μL pipette tip. Images were acquired using a Cellvivo microscope (Olympus). Data was processed using Fiji.

Raman microscopy. The Raman spectra of the organoids were acquired by using a Renishaw inVia Confocal Raman spectrometer connected to a Leica microscope (Leica DMLM, Leica Microsystems, Buffalo Grove, IL, USA). A 785 nm near-IR laser, Nikon Flour 60×NA=1.00 water immersion objective lens, and 1,000 milli-second exposure time with the average number of 100 accumulations were used for the data acquisition of each scanning position of the organoids. To circumvent a strong background signal, a quartz slide (Chemglass Life Sciences, NJ, USA) was used as a substrate for the Raman spectra acquisition. Organoids were collected on Day 30 for analysis. Optical coherence tomography. A Spectral-Domain Optical Coherence Tomography (SD-OCT) system similar to previous work^{128, 129} was used for label-free longitudinal imaging of the heart organoids. A superluminescent diode (EXALOS, EXC250023-00) was used as the light source with a center wavelength of ~1300 nm and a 3 dB spectrum range of ~180 nm. A spectrometer (Wasatch Photonics, Cobra 1300) based on a 2048-pixel InGaAs line-scan camera (Sensors Unlimited, GL2048) was used to provide a maximum A-scan rate of 147 kHz. A 5× objective lens was used and the transverse and axial resolutions were measured to be ~2.83 μm and ~3.04 μm in tissue, respectively. Longitudinal 3D OCT imaging was performed every other day from Day 20 to Day 30. Each 3D OCT scan comprised 600 A-scans per B scan and 600 B-scans. Each organoid requires ~22 seconds for image acquisition using an exposure time of ~40 μs for each A-scan. Eight organoids from each group were imaged and used for analysis. The media level in each well was adjusted during imaging to reduce image artifacts and minimize light absorption. Re-scaling of acquired OCT images was performed using ImageJ to obtain isotropic pixel size in x-y-z dimensions (Schneider et al., 2012). Registration of the same organoid at different days, cavity segmentation, and 3D rendering were performed using Amira software (Thermo Fisher Scientific). The total volume and cavities inside the organoids were quantified from the segmentation data.

Ondansetron treatment. Ondansetron hydrochloride (Sigma) was prepared at 200 μM in DMSO and was further diluted in DMEM/F12 ((Dulbecco's Modified Eagle Medium/Nutrient Mixture F-12) before being sterile filtered via a 0.22 μm PVDF filter (Sigma). Ondansetron was applied to heart organoids at final concentrations of 1 μM, 10 μM, and 100 μM in EMM2/1 medium, and was applied from Day 9 to Day 20 of culture. Organoids were collected on Day 30 for analysis.

Lentiviral Transduction. HEK293T (Horizon Inspired Cell Solutions) cells were transfected with the Flip-GFP plasmid (VectorBuilder) and the packaging plasmids pMD2 and psPAX2 using lipofectamine with Plus reagent (Thermo) to create a lentivirus. The lentivirus was added to iPSC-L1 cells with 8 μg/ml polybrene (Fisher Scientific) and incubated overnight. Puromycin selection was carried out for 3-5 days until all cells lacking lentivirus were absent from the well. Surviving clones were selected, collected, replated, and further expanded to give rise to the FlipGFP line.

Doxorubicin treatment. Doxorubicin hydrochloride (Sigma) was diluted to 1 mM in DMEM/F12 and was applied to heart organoids at a final concentration of 10 μM for 48 hours from Day 28 to Day 30 of culture.

DEAB and Retinoic Acid treatment. 4-Diethylaminobenzaldehyde (DEAB) (Sigma) was prepared at 1 M in DMSO, diluted further to 10 mM using DMSO, then finally diluted to 1 mM in DMEM/F12. Retinoic Acid (RA) (Sigma) was prepared at 1 M in DMSO, and diluted to 100 μM in DMEM/F12. Diluted solutions of DEAB and RA were sterile filtered via 0.22 μm PVDF filters (Sigma). DEAB was applied to heart organoids at a final concentration of 10 μM. RA was applied to heart organoids at a final concentration of 1 μM. DEAB, RA, and DEAB+RA were applied to heart organoids from Day 20 to Day 30 of culture using the EMM2/1 strategy. Organoids were collected on Day 30 for analysis.

Agilent Seahorse Metabolic Assay. An Agilent Seahorse XFe96 (Agilent) was used to perform real-time extracellular flux assays. The day before the assay, 200 μL of XF Calibrant was loaded into each well of the 96-well utility plate included with the sensor cartridge and the sensors were submerged in a 37° C. non-CO₂ incubator overnight. Also the day before the assay, poly-lysine (Sigma) was used to coat XFe96 spheroid microplates. In brief, poly-lysine was prepared at 100 μg/mL in water and 30 μL of this solution was added to each well of the microplate. After sitting for 20 minutes, the poly-lysine solution was aspirated from the wells and washed two times with sterile water. Then, the plate was allowed to air dry for a minimum of 30 minutes. Then, the plate was warmed for 30 minutes in a 37° C. non-CO₂ incubator for 30 minutes. Finally, 100 μL of 37° C. DMEM/F12 was added to each well of the microplate and the microplate was returned to a 37° C. non-CO₂ incubator overnight. The following steps describe actions performed on the day of the assay, in order. XF RPMI was prepared (phenol red-free) (Agilent) was used as the base medium of the assay which was supplemented with 1 mM pyruvate (Agilent), 2 mM glutamine (Agilent), 11.1 mM glucose (Gibco), and 12.2 μM L-Carnitine (Sigma). Using this prepared XF RPMI, drug solutions from the Cell Mito Stress Test kit (Agilent) were resuspended, vortexed for 1 minute, and let rest at room temperature for 1 hour. In this time, the poly-lysine-coated XFe96 spheroid microplates were removed from the incubator and the DMEM/F12 was removed from the plate, washed 1× with 166 μL of prepared XF RPMI, and finally, 175 μL of prepared XF RPMI was added to each well. Then, day 30 organoids in each condition were washed with 166 L of prepared XF RPMI two times and were transferred to the XFe96 spheroid microplate coated with poly-lysine. Organoids were transferred to the wells using a cut p200 pipette tip. It was ensured that organoids were centered in the well. Following this, the plate was placed in a 37° C. non-CO² incubator for 1 hour. Drug solutions (Oligomycin, FCCP, and Rot/AA) were loaded into ports A, B, and C, respectively. Port concentrations of oligomycin, FCCP, and Rot/AA were 25 μM, 20 μM, and 20 μM, respectively, such that their final concentrations in solution were 2.5 μM, 2 μM, and 2 μM, respectively. The assay was configured such that the baseline phase ran for 6 cycles, and the oligomycin, FCCP, and Rot/AA stages ran for 10 cycles each. Each cycle constituted a 3-minute mixing, a 0-minute waiting, and a 3-minute measuring phase. Data was normalized to organoid area.

Statistics and reproducibility. Microsoft Excel was used to collect raw data. Graphpad Prism 9 software was used for all analyses. Data presented as normal distribution. Statistical significance was evaluated using one-way ANOVA with Dunnett or Brown-Forsyth and Welch post-test corrections, or using unpaired t-tests, when appropriate (p<0.05). All data presented as mean±s.e.m. Statistical methods are specifically indicated in figure legends. Number of independent organoids used for every quantification and every statistical test is indicated in figure legends. When more than one independent experiment (plate/batch of organoids) was performed (a large majority of data in this manuscript), this is indicated appropriately in the figure legend.

### Results

### Extended Heart Developmental Modeling Through Improved Developmental Induction Strategies

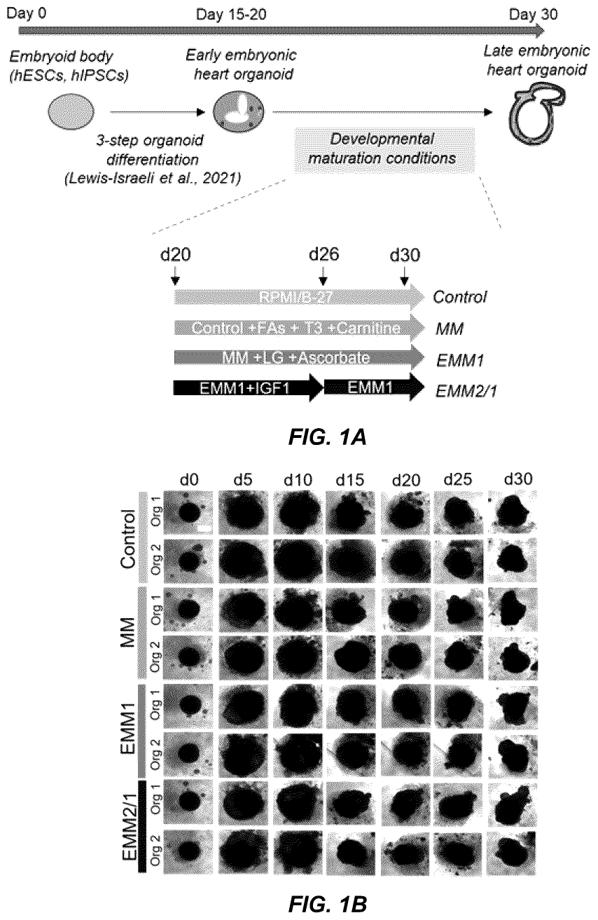

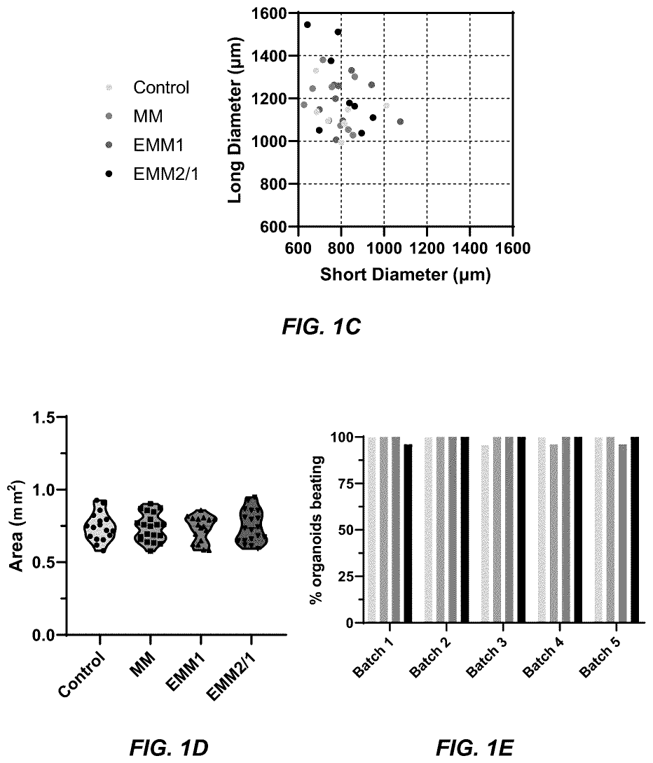

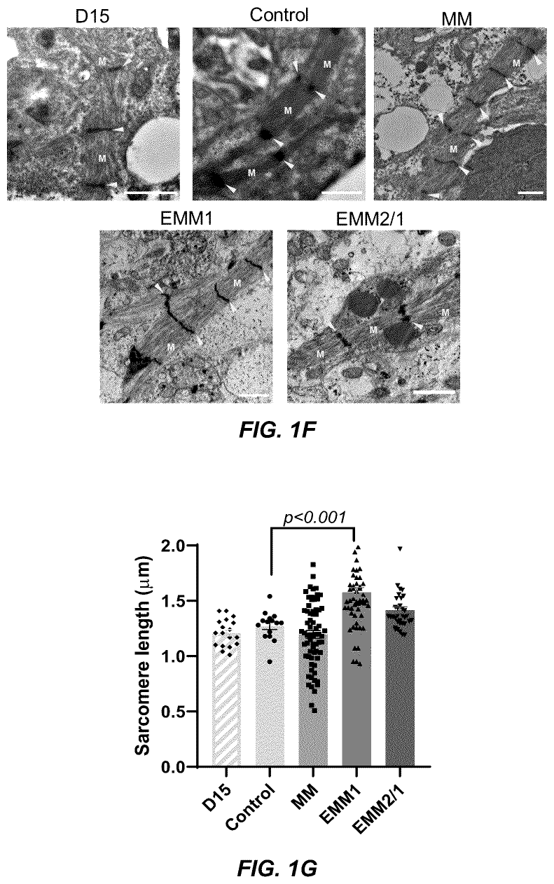

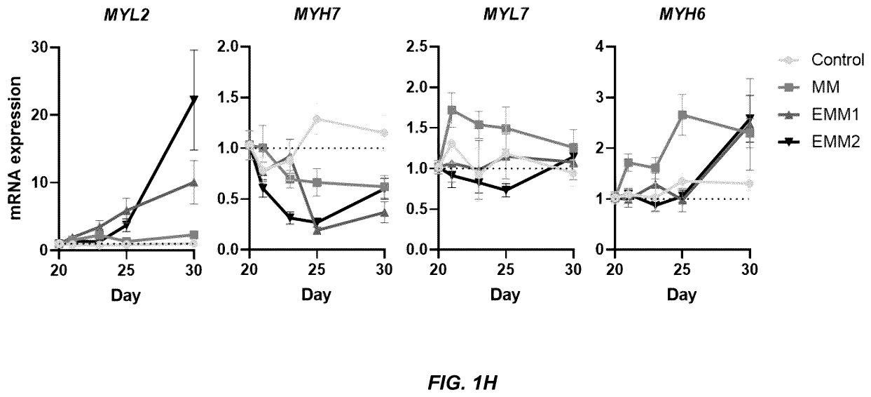

A detailed protocol for the generation of self-organizing early embryonic human heart organoids has been previously described¹⁵ and this protocol constituted the starting step for the methodology described below. In brief, heart organoids were differentiated from hiPSC embryoid bodies to the cardiac lineage between days 0 and 7 through a timewise 3-step Wnt pathway modulation strategy, and then cultured until day 20 in RPMI¹⁵. To examine the effect of more advanced organoid culture strategies mimicking in utero conditions on heart organoid development, day 20 early embryonic-like heart organoids had four different developmental induction strategies from day 20 to day 30 implemented on them (FIG. 1A). These strategies represent gradual increasing steps in complexity relevant to in utero conditions (in order of less complex to more complex: control, maturation medium, enhanced maturation medium 1, enhanced maturation medium 2/1) in comparison to previous human and animal developmental studies²⁰⁻²⁴. The “control strategy” represents a continuation of organoid culture in the base medium used for organoid formation, RPMI/B27. The “maturation medium (MM) strategy” used RPMI/B27 with added fatty acids (an embryonic relevant concentration of oleic acid, linoleic acid, and palmitic acid)^{23,24} and L-carnitine²⁵ to facilitate a developmentally relevant transition from glucose utilization to fatty acid metabolism characteristic of the fetal human heart²⁶⁻³⁰. The MM strategy also used T3 hormone, a potent activator of organ growth during embryonic development and metabolic maturation, which has been shown to stimulate cardiovascular growth^{31,32}. The “enhanced maturation medium 1 (EMM1) strategy” used the same basal composition as MM but decreased the concentration of glucose to cardiac physiological levels³³⁻³⁵ (from 11.1 mM to 4 mM to further encourage the transition to fatty acid oxidation) and added ascorbic acid as reactive oxygen species scavenger to counteract the increased oxidative stress^{36,37}. “Enhanced maturation medium strategy 2/1 (EMM2/1)” utilized a combination of two different media formulations. From days 20-26, EMM2 media was utilized and was the same basal composition as EMM1 with added IGF-1. IGF-1 plays important roles during embryonic and fetal development in tissue growth and maturation, especially in the heart, as proven in murine and human studies³⁸⁻⁴⁰. From day 26 onwards, EMM1 media was utilized in the EMM2/1 strategy. The EMM2/1 strategy represents the most advanced condition and mimicked in utero heart development to the greatest extent. More detailed descriptions of all developmental induction strategies along with concentrations of respective media formulations can be found in the Materials and Methods section above.

Heart organoids treated with the different developmental induction strategies continued to grow and develop, with drastic changes in morphology depending on condition (FIGS. 1B-1D). Organoids experienced a period of rapid growth from day 0 to day 10, increasing in diameter while retaining their spherical structure (FIG. 1B) and continuing to grow until day 30. Organoids developed distinct elliptical morphologies after day 20, elongating and contorting as observed by brightfield microscopy and growing to possess long diameters between 1000 and 1600 μm, while short diameters ranged from 600 to ~1000 μm on day 30 (FIGS. 1B-1C). Organoid area measured by brightfield microscopy revealed similar trends for each condition, between 0.6 mm² to ~0.9 mm² (FIG. 1D). By day 30 of culture, nearly 100% of organoids in every condition were observed to be beating across five independent experiments (n=22-24 organoids per condition per experiment) (FIG. 1E). Transmission electron microscopy (TEM) images indicated the presence of well-developed myofibrils and the formation of sarcomeres within the organoids (FIG. 1F) in all conditions, with sarcomeres in the EMM1 condition displaying a significantly increased sarcomere length of 1.58±0.323 μm relative to control (FIG. 1G). qRT-PCR revealed the expression of hallmark cardiomyocyte sarcomere genes from day 20 to day 30 as expected. Interestingly, different conditions displayed differential expression of MYL2, MYL7, MYH7, and MYH6 at various timepoints of development, indicating that our developmental maturation strategies elicit distinct transcriptomic effects on heart organoid growth (FIG. 1H).

Single Cell RNA Sequencing (scRNA-Seq) of Human Heart Organoids Under Developmental Induction Reveals Cell Type Complexity and Differences in Cellular Compositions

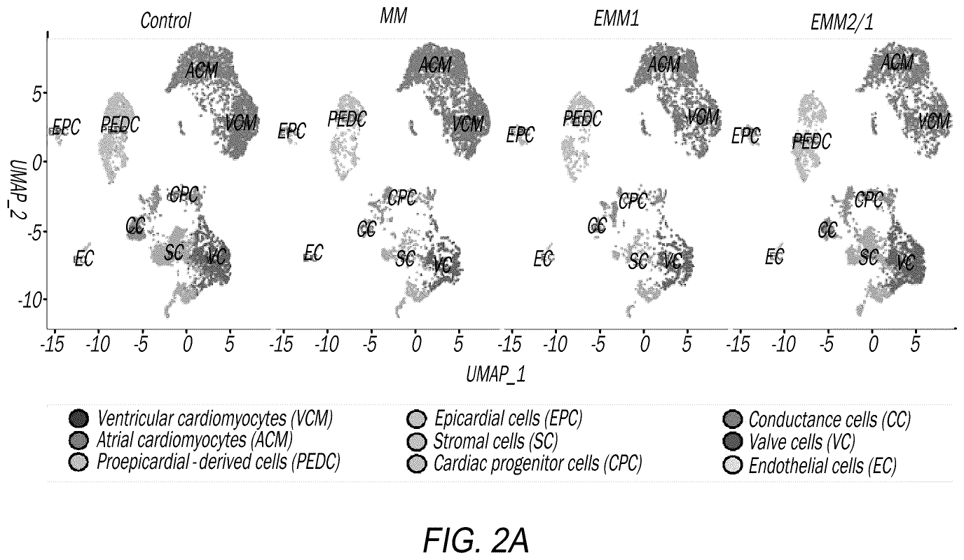

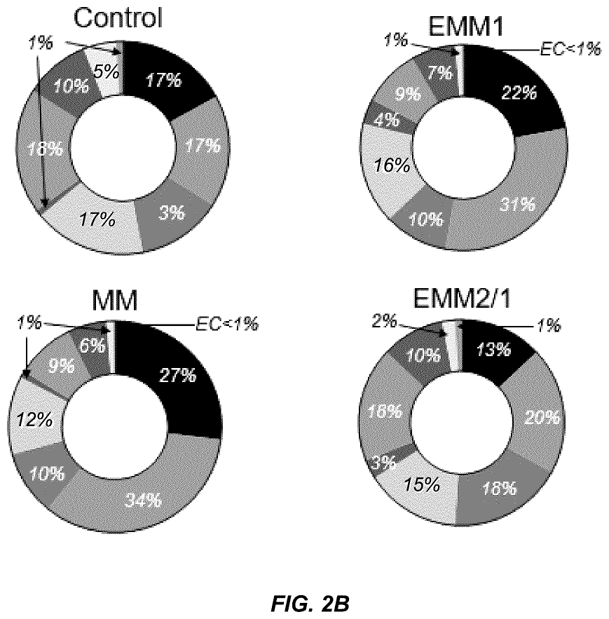

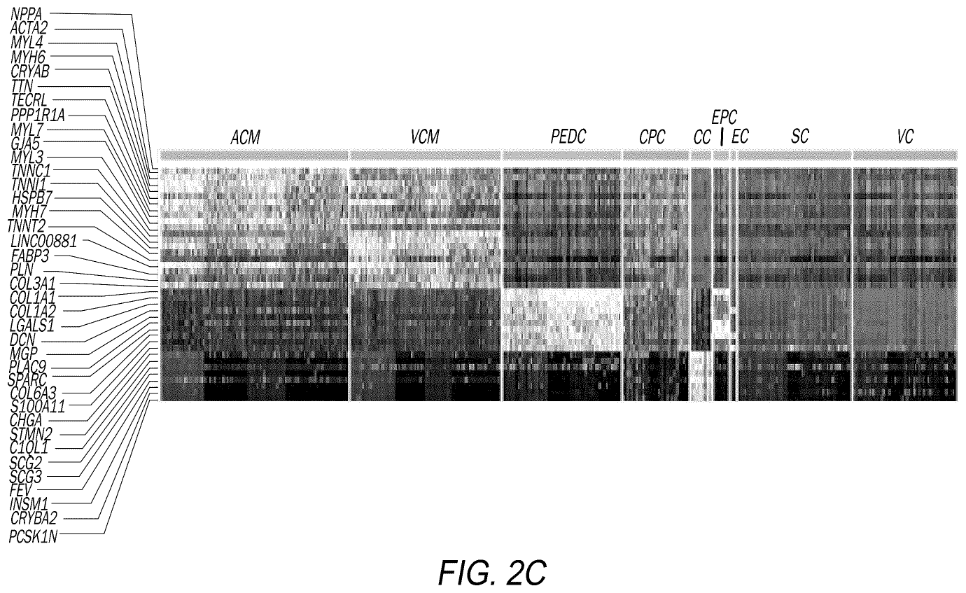

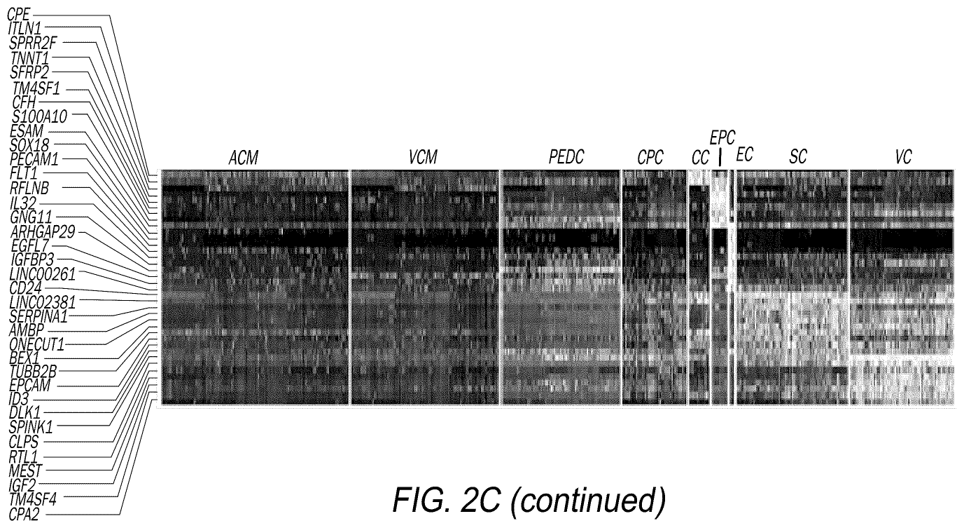

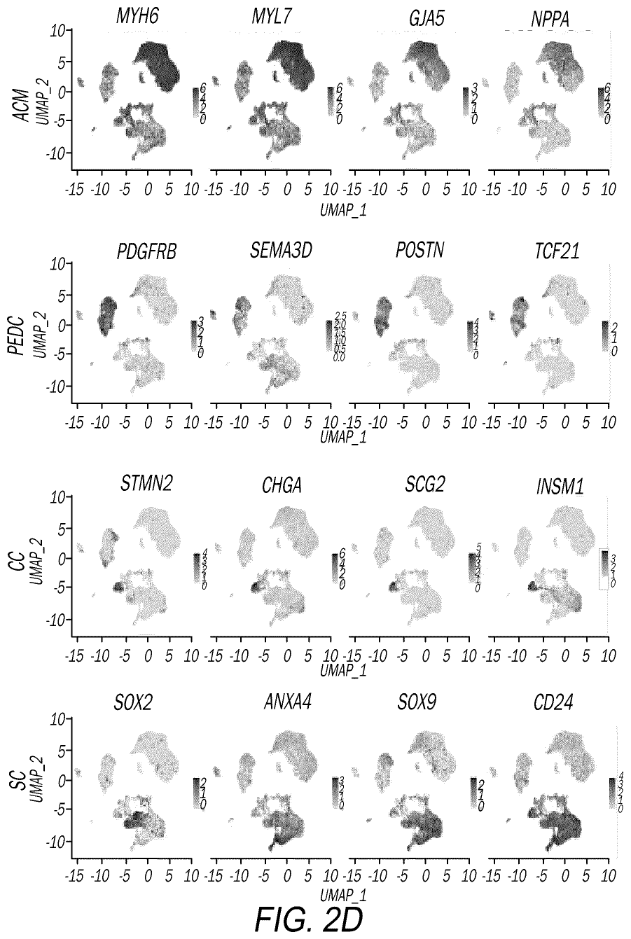

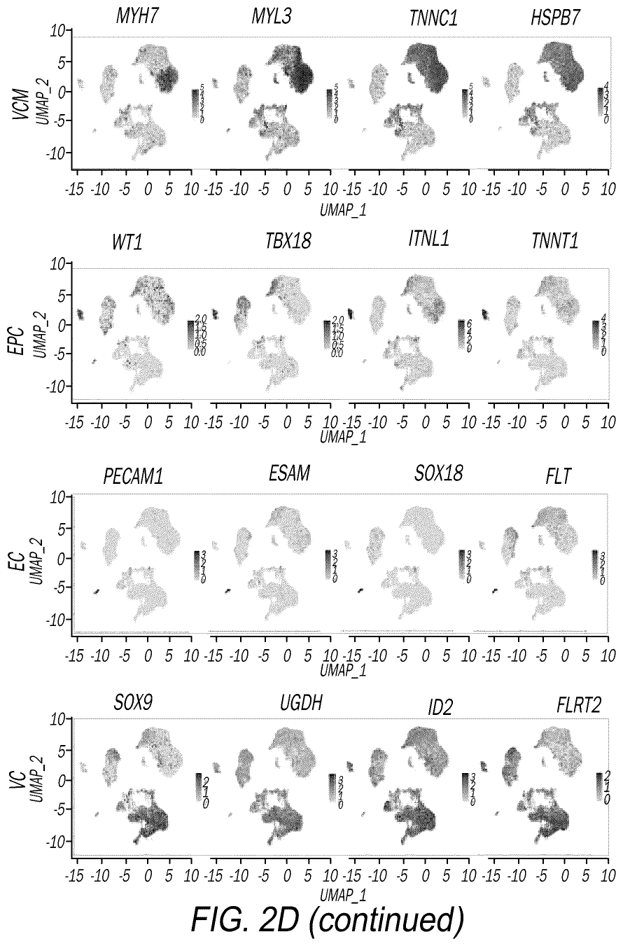

To characterize the cellular and transcriptomic composition of heart organoids in each of the developmental induction conditions, scRNA-seq at day 34 of organoid culture was performed. UMAP projections display unsupervised K-means clustering analyses for each condition (FIG. 2A). Ventricular and atrial cardiomyocytes (VCMs and ACMs, respectively), valve cells (VCs), proepicardial derived cells (PEDCs), epicardial cells (ECs), stromal cells (SCs), cardiac progenitor cells (CPCs), conductance cells (CCs), and endothelial cells (ECs) were revealed in all conditions of the heart organoids. The abundance of several significant cell groups varied according to the developmental medium conditions. Control organoids were composed of 17% VCMs, 17% ACMs, 3% VCs, 17% PEDCs, 1% EPCs, 18% SCs, 10% CPCs, 5% CCs, and 1% ECs (FIG. 2B). Relative to control, MM organoids displayed an increased percentage of both VCMs and ACMs (27% and 34%, respectively), increased VCs (10%), decreased PEDCs (12%), 1% EPCs, decreased SCs (9%), decreased CPCs (6%), and decreased CCs (1%). Relative to control, EMM1 organoids contained an increased percentage of VCMs (22%), increased ACMs (31%), increased VCs (10%), decreased PEDCs (16%), increased EPCs (4%), decreased SCs (9%), decreased CPCs (7%), and decreased CCs (1%). Relative to control, EMM2/1 organoids exhibited a decreased VCM percentage (13%), increased ACMs (20%), increased VCs (18%), decreased PEDCs (15%), increased EPCs (3%), 18% SCs, 10% CPCs, and decreased CCs (2%). Differential gene expression analyses determined signature genes which were used to identify clusters (FIGS. 2C-2D). ACMs possessed high expression of MYH6, MYL7, NPPA, and GJA5^{4,41-43}. VCMs displayed high expression of MYL3, MYH7, TNNC1, and HSPB7^{41,42,44-47}. PEDCs showed high expression of PDGFRB, SEMA3D, POSTN, and TCF21⁴⁸⁻⁵². EPCs shared slight similarity with PEDCs, yet also presented differentially expressed genes including WT1, TBX18, ITLN1, and TNNT1^{41,53-56}. CCs displayed high expression of STMN2, CHGA, SCG2, and INSM1; genes that are involved in neuron growth, development and neuroendocrine signaling⁵⁷⁻⁶² and that share similarity with human embryonic heart datasets in a neural crest cell and Schwann cell cluster⁴¹. ECs possessed high expression of PECAM1, ESAM, SOX18, and FLT^{41,63-66}. SCs were identified by expression of SOX2, ANXA4, SOX9, CD24^{41,67-71}. VCs were identified via the expression of SOX9, UGDH, ID2, and FLRT2^{42,69,72-78}. Taken together, these results show that heart organoids possess similar cell types as those that are found in the developing human heart, and suggest, in agreement with previous studies on cardiac development^{41,42}, that by day 20 the main cardiac cell lineages are already determined and that developmental induction conditions can exert dramatic effects on the expansion and maturation of these cell types to better reflect in vivo heart development.

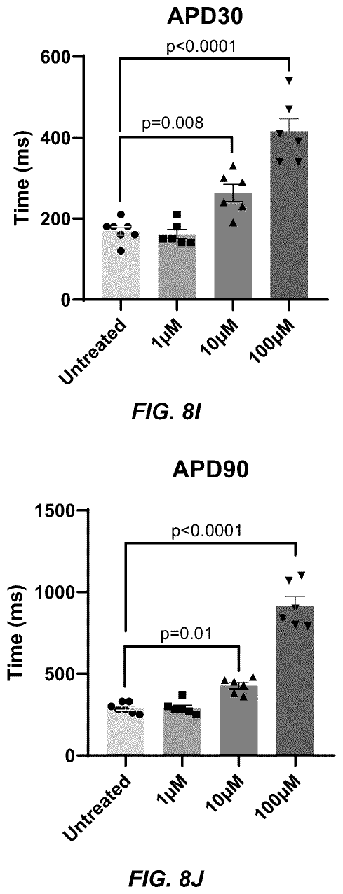

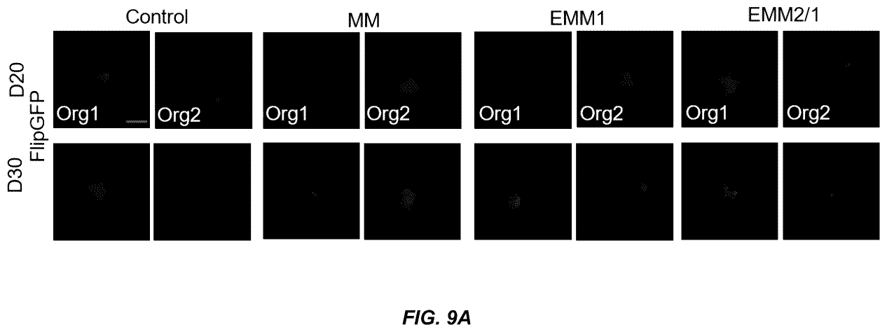

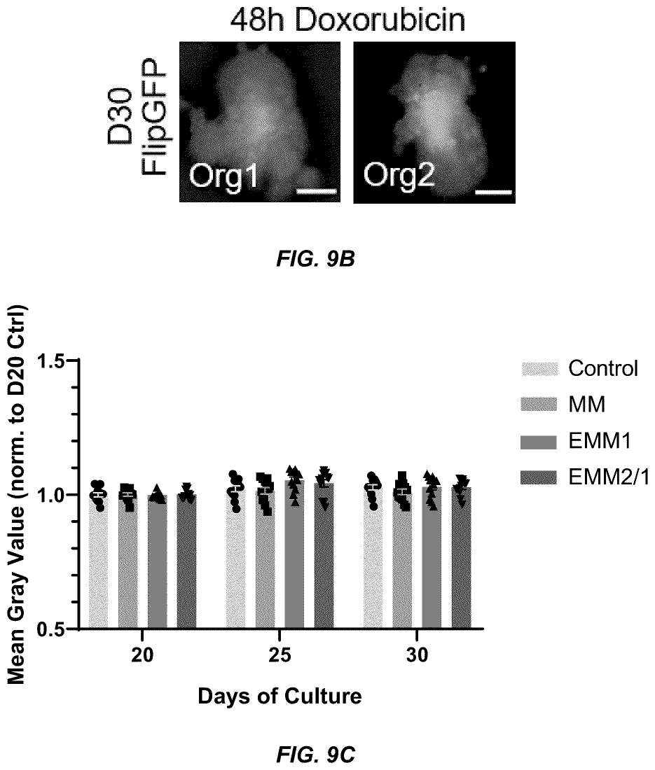

To investigate if differences in cell type proportions in organoids between conditions were caused by apoptosis, a genetic reporter iPSC line named FlipGFP was created that fluoresces when the active form of caspase 3, a master regulator of apoptosis, is present⁷⁹. Very low levels of apoptosis in heart organoids from day 20 to day 30 (FIG. 9A) were found as well as no differences in apoptosis levels between conditions (FIG. 9B). A 48-hour doxorubicin treatment was used as a positive control and displayed high levels of fluorescence (FIG. 9C). This data suggests that cell type proportions in organoids from different conditions is not driven by apoptosis.

A variety of additional, specific marker gene groups were identified in the heart organoid datasets. Cardiac fibroblasts were identified within the PEDC cluster, showing expression of DCN, LUM, OGN, and POSTN, and COL1A1^{41,80} (data not shown). Organoids also recapitulated key genes involved in left-right asymmetry in all conditions such as PITX2, PRRX2, LEFTY1 and PRRX1⁸¹⁻⁸⁴ (data not shown). Additionally, organoids displayed high upregulation of proliferation markers such as MKI67, PCNA, AURKB, and CDK1 in all conditions, indicating that important growth and remodeling are still undergoing at day 34 of differentiation⁸⁵⁻⁸⁷ (data not shown). Cells of the first heart field (FHF) and the second heart field contribute to linear heart tube expansion and subsequent chamber formation and are important for proper cardiac morphogenesis⁸⁸. Various FHF and SHF markers were observed in organoids in all conditions^{89,90} (data not shown). HAND1, HAND2, TBX5, and HCN4 were all upregulated in the VCM and ACM clusters for all conditions. ISLI was upregulated in the VCM and ACM clusters as well as the CC cluster for each condition. In addition, outflow tract markers such as RSPO3⁹¹ and WNT5A^{92,93} were upregulated in the PEDC, ACM, VCM, and SC clusters for all conditions (data not shown).

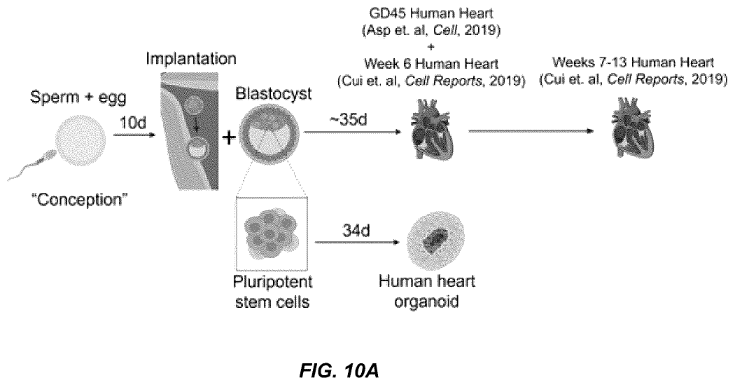

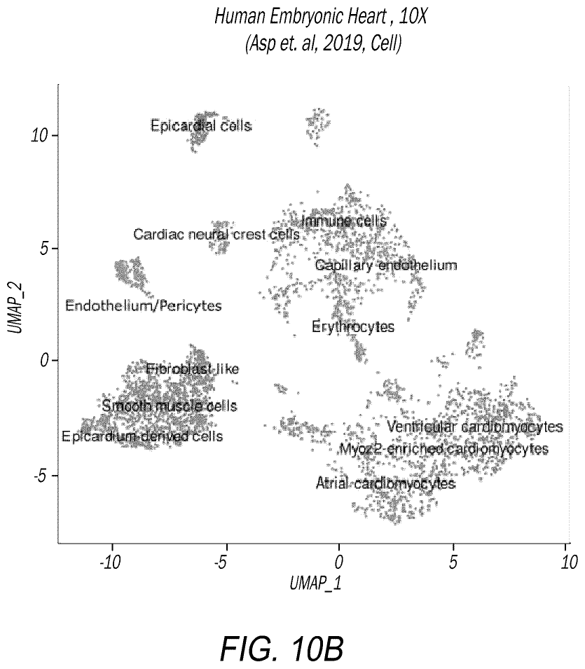

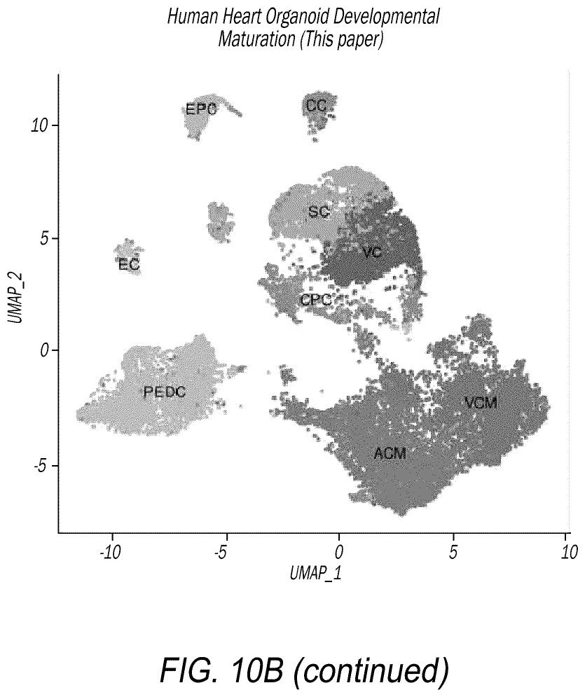

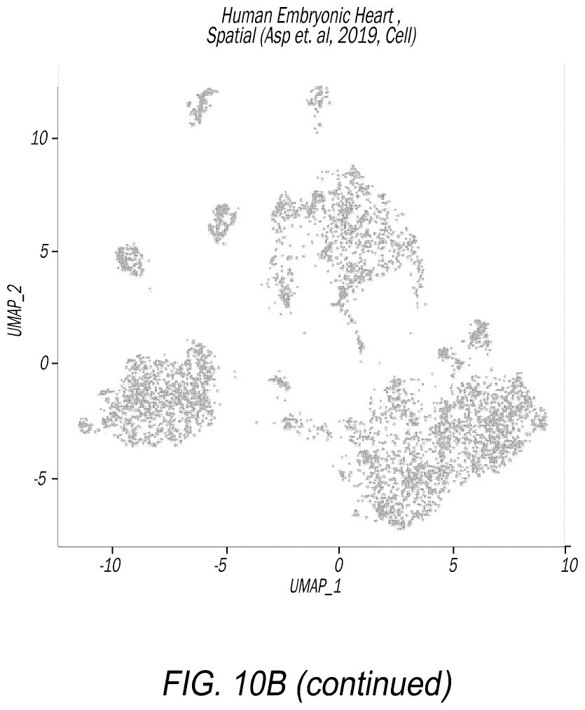

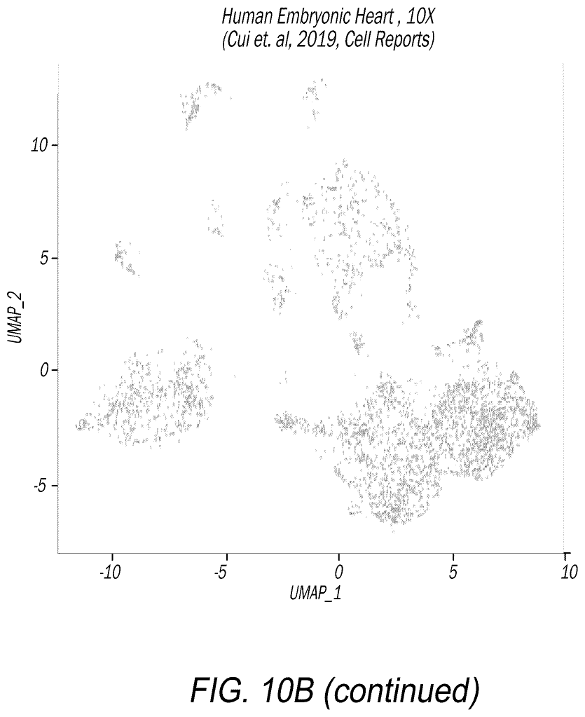

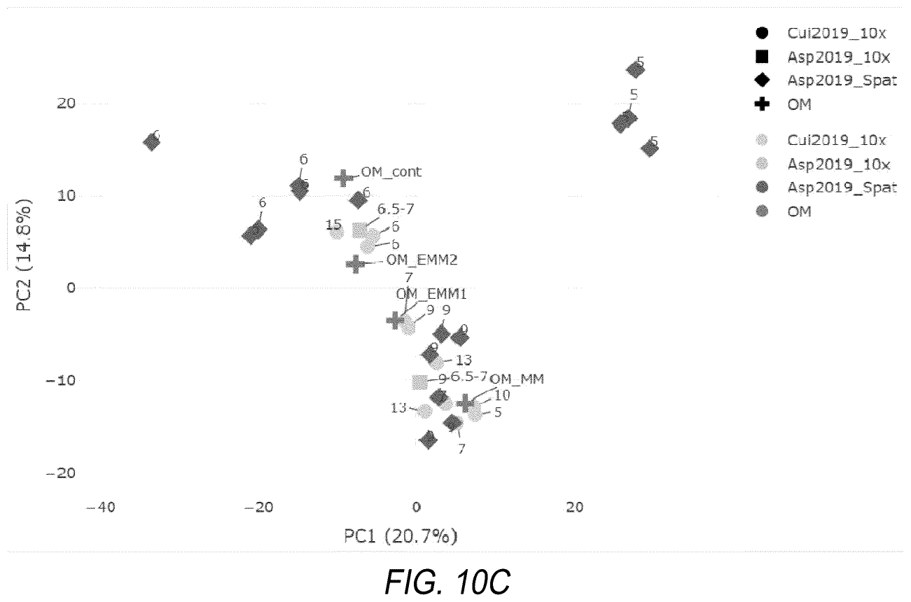

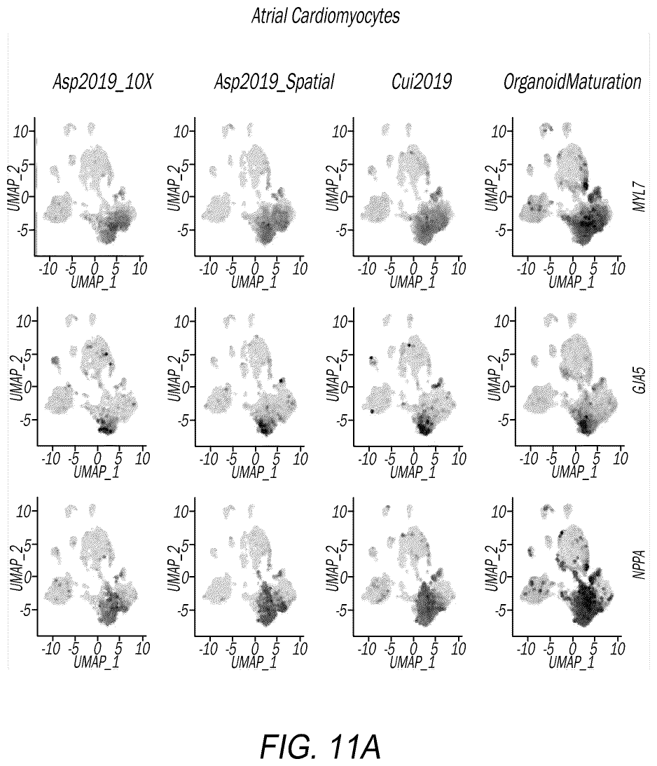

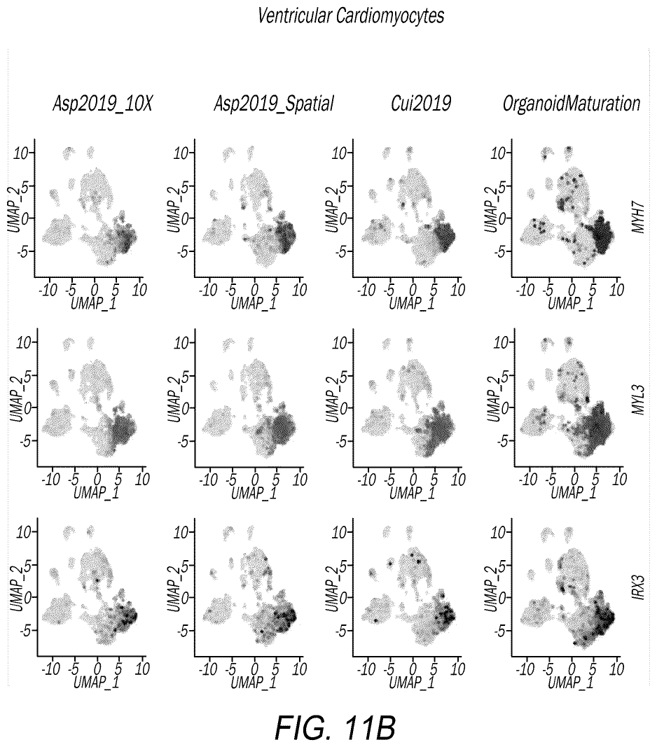

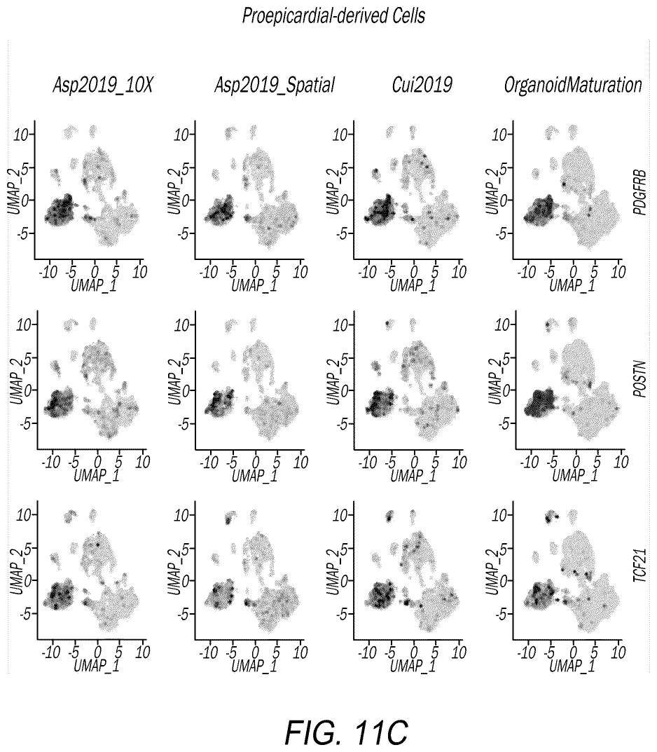

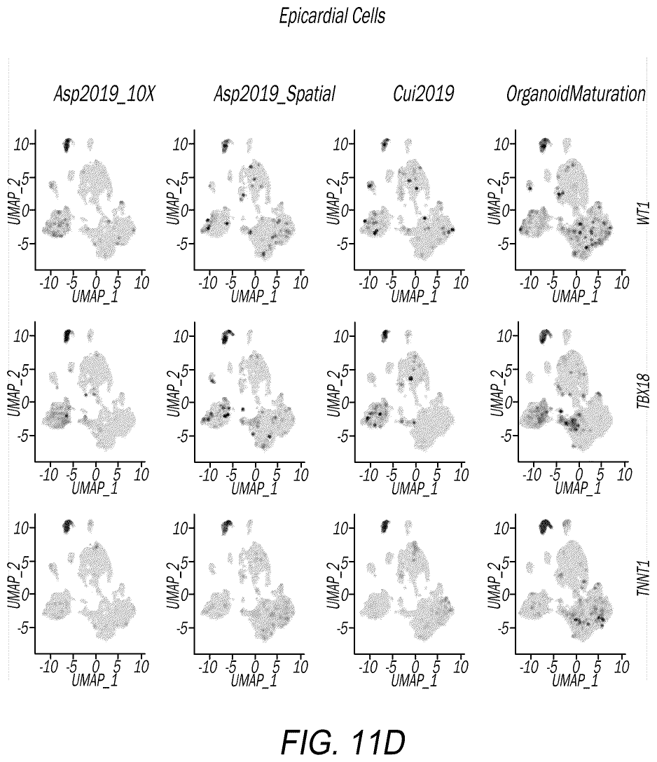

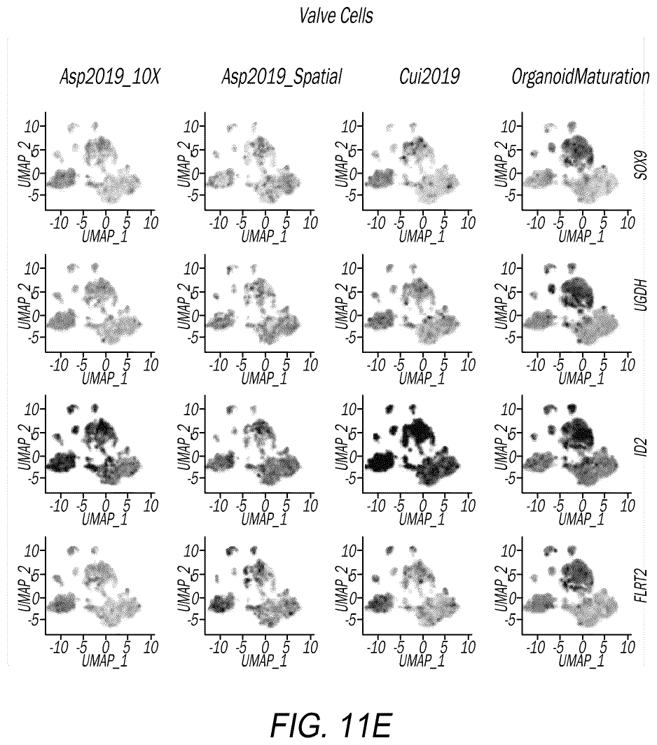

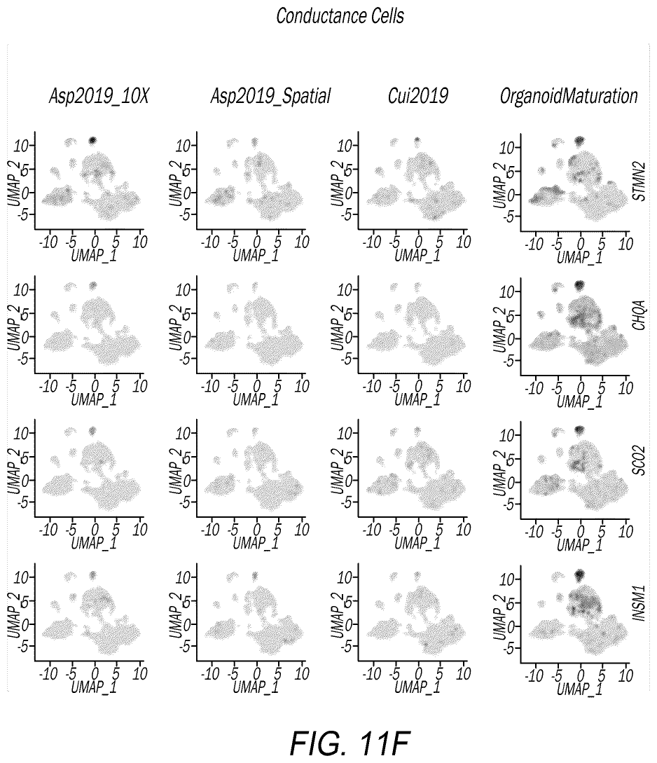

Extending these analyses, publicly available data from the Human Cell Atlas project⁴¹ was utilized from gestational day 45 (GD45) and from human embryonic hearts from 5 weeks to 13 weeks of gestation⁴² to compare the human heart organoids to that of developing human hearts (FIGS. 10A-10C). Based on their time in culture, human heart organoids should be closest to GD45 or 6-7 gestational week human fetal hearts (FIG. 10A). These scRNAseq datasets were integrated and high overlap between cell type annotations and that presented from the Human Cell Atlas project was discovered (FIG. 10B), with atrial and ventricular cardiomyocytes, proepicardial-derived cells (named fibroblast-like, smooth muscle cells, and epicardium-derived cells in the Human Cell Atlas project dataset), endothelial cells, and epicardial cells displaying a high degree of clustering between datasets. Interestingly, the valve cells mapped closely to capillary endothelial cells, our stromal cells mapped closely to immune cells, and the conductance cells did not have a clear mapping correlation, even though the conductance cell cluster displays similar gene expression profiles to the cardiac neural crest cluster in the Human Cell Atlas project dataset⁴¹. These datasets were then used to compare gene expression profiles at the single-cell level (FIG. 10C and FIGS. 11A-11F). Using the top 1000 differentially expressed genes in each dataset from the VCM, ACM, PEDC, and EPC-mapped regions, a high degree of similarity between organoids from each condition and that from embryonic hearts (FIG. 10C) was shown, with Control organoids and EMM2/1 organoids clustering closely to week 6 embryonic hearts, whereas MM and EMM1 organoids clustered closer to weeks 7-9 embryonic hearts, which may indicate that the MM and EMM1 maturation strategies accelerate the developmental transcriptomes in the organoids at a rate that surpasses traditional developmental paradigms compared to Control and EMM2/1 organoids, which stay true to anticipated developmental stages. Also assessed was individual gene expression levels in clusters across embryonic hearts and human heart organoids and a high degree of similarity across all major clusters was shown (FIGS. 11A-11F).

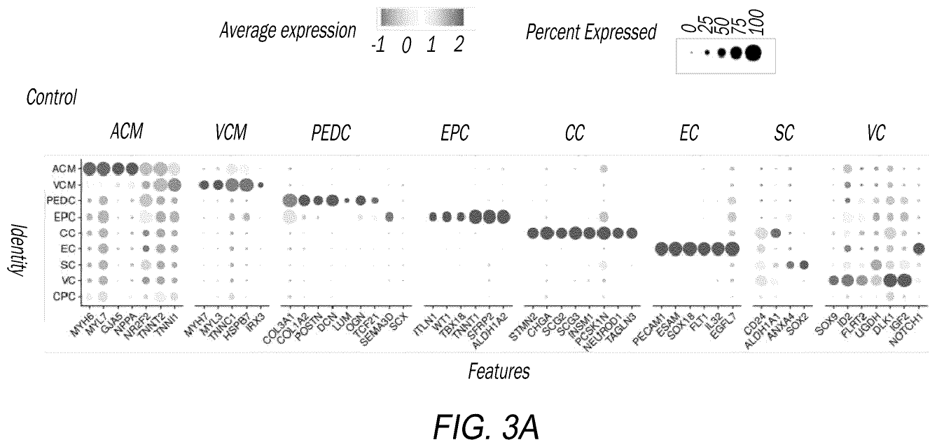

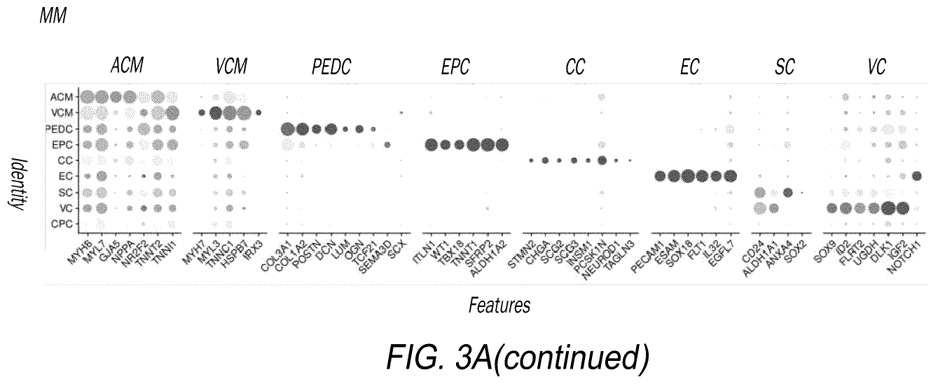

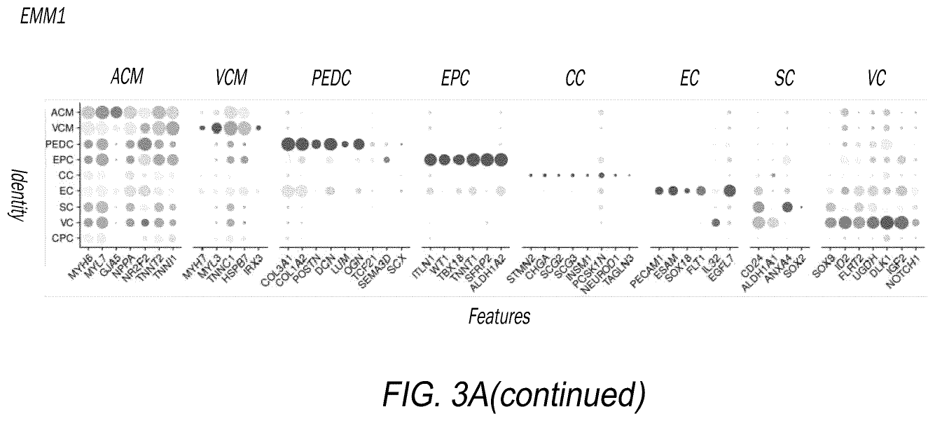

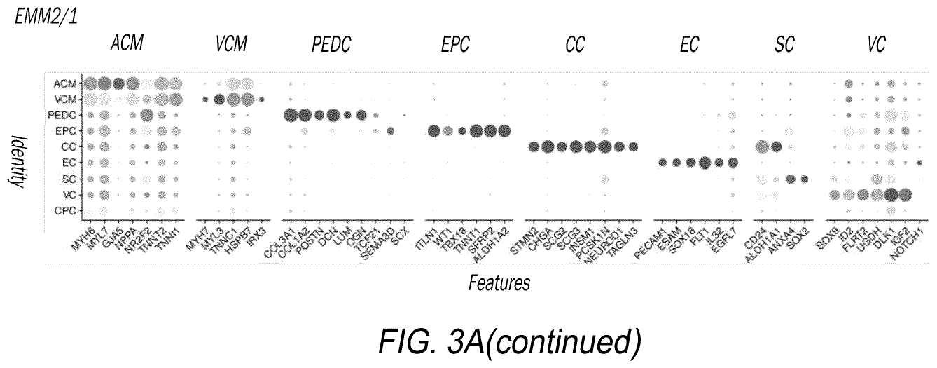

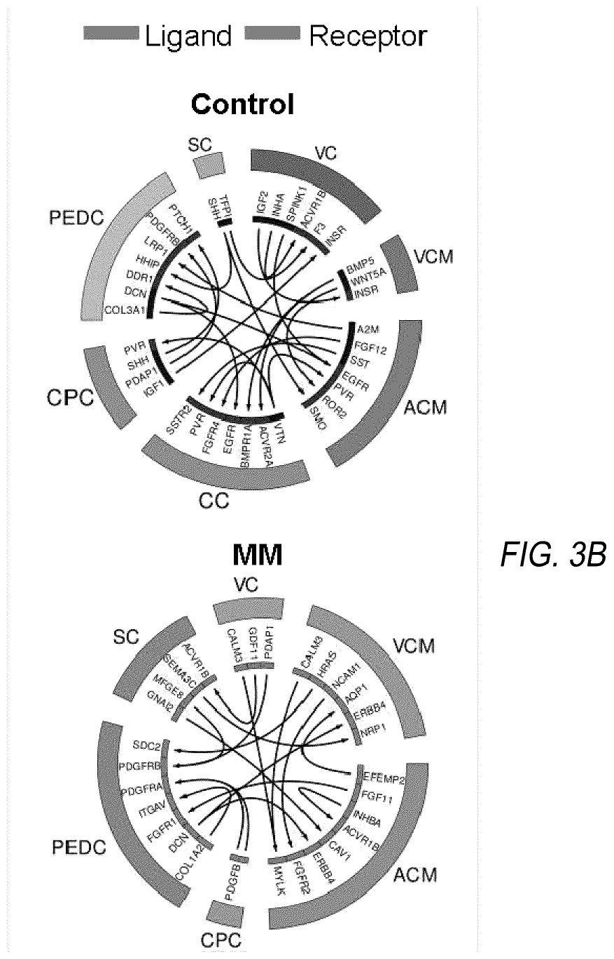

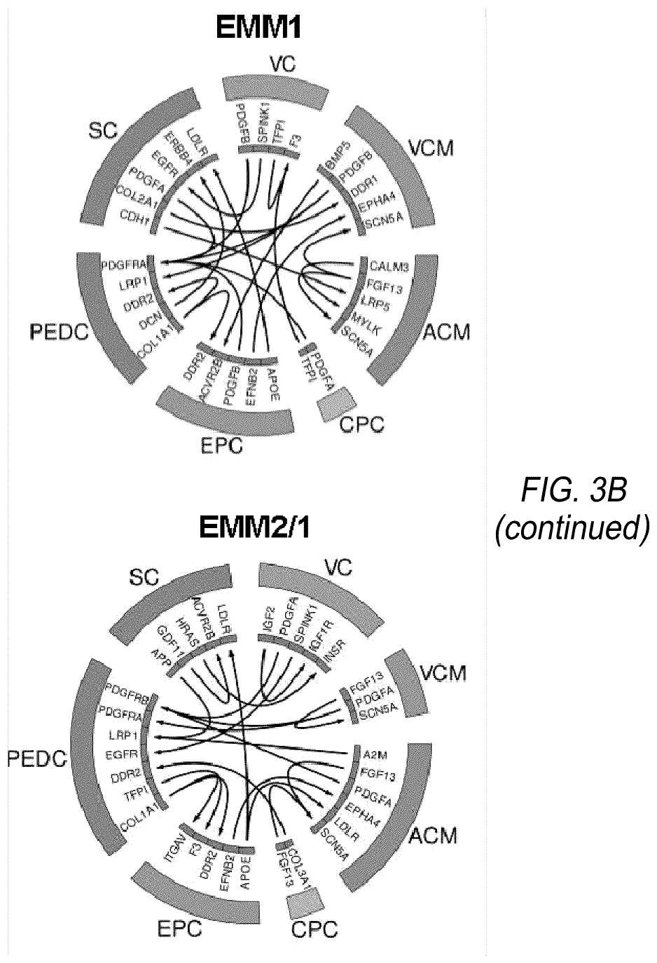

To complement the above scRNA-seq analyses, dot plots describing the average and percent expression of key lineage-defining differentially expressed genes for individual clusters are depicted for each developmental induction condition illustrating the cellular complexity of the obtained heart organoids at day 34 (FIG. 3A). As has been shown before^{13-15,17,94}, the high cellular complexity of the organoids drives self-organization and cell-cell communication. Computational analysis of cell-cell communication networks for key genes found in the organoids was performed. Various complex receptor-ligand communication pathways were identified within the obtained human heart organoids in each condition (FIG. 3B). Receptor-ligand networks include JAG1-NOTCH1, PDGFRs, IGF2-IGF2R, INSR, and VEGF, among others. Gene Ontology (GO) analysis was also performed for biological process terms corresponding to top differentially expressed genes contributing to the ontology for each cluster, as well as top shared genes between all four conditions per cluster (data not shown). To further investigate cell-cell communication networks, scRNA-seq data was utilized to highlight key receptor-ligand pairs within the organoids from each maturation condition (data not shown). This data highlights the ability and sensitivity of the obtained organoids to respond to various developmental maturation stimuli surrounding cell-cell communication paradigms.

### Mitochondrial Maturation and Oxidative Metabolism in Human Heart Organoids Under Developmental Induction Conditions

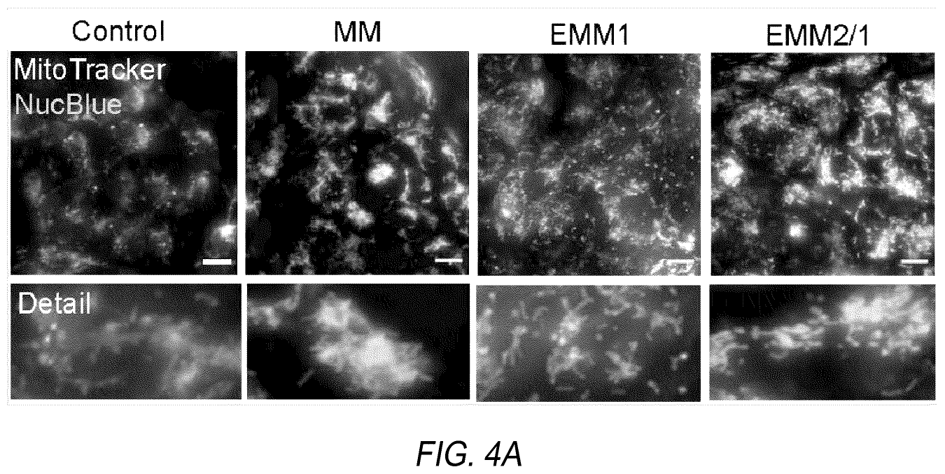

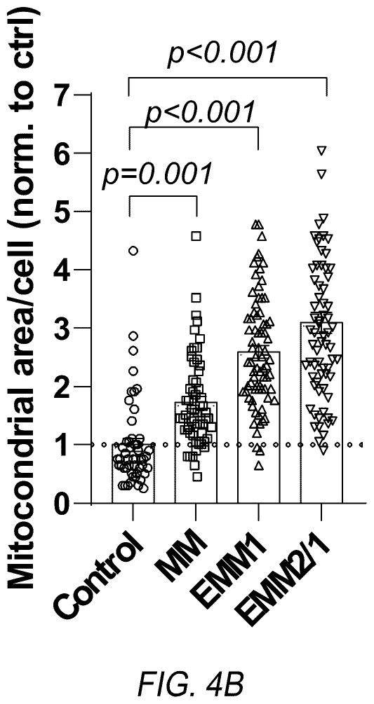

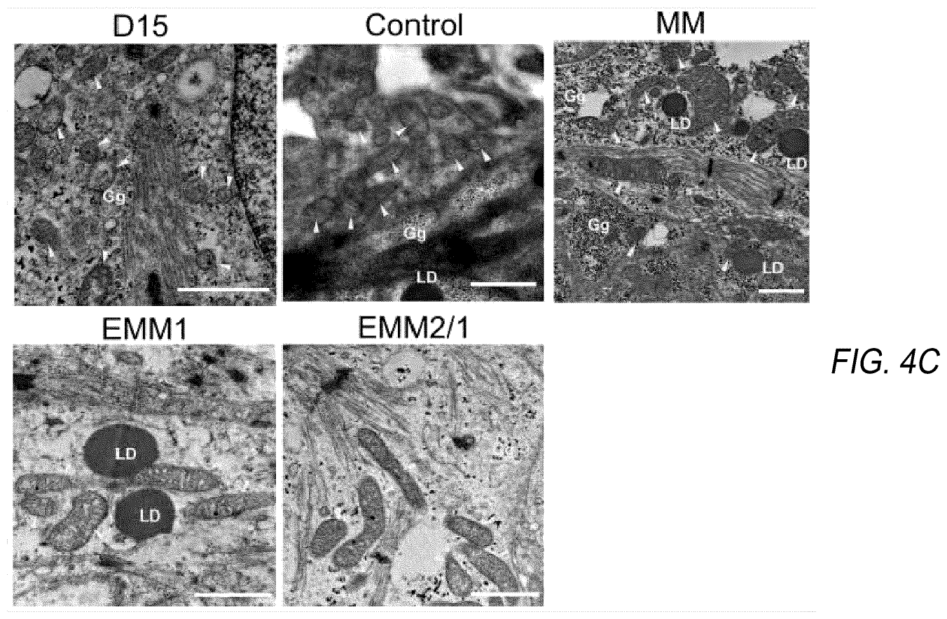

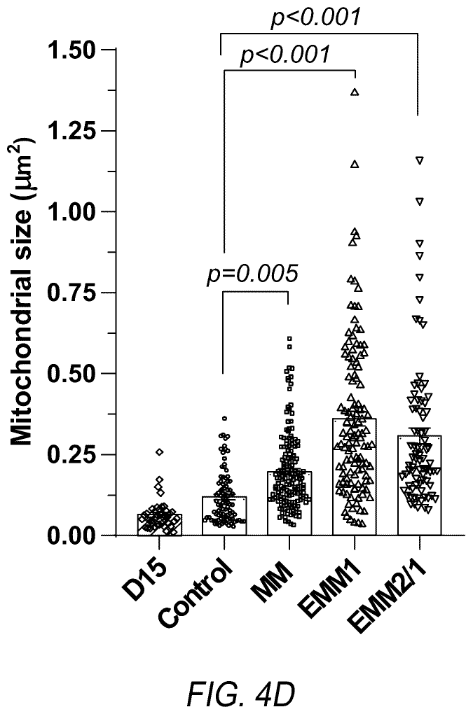

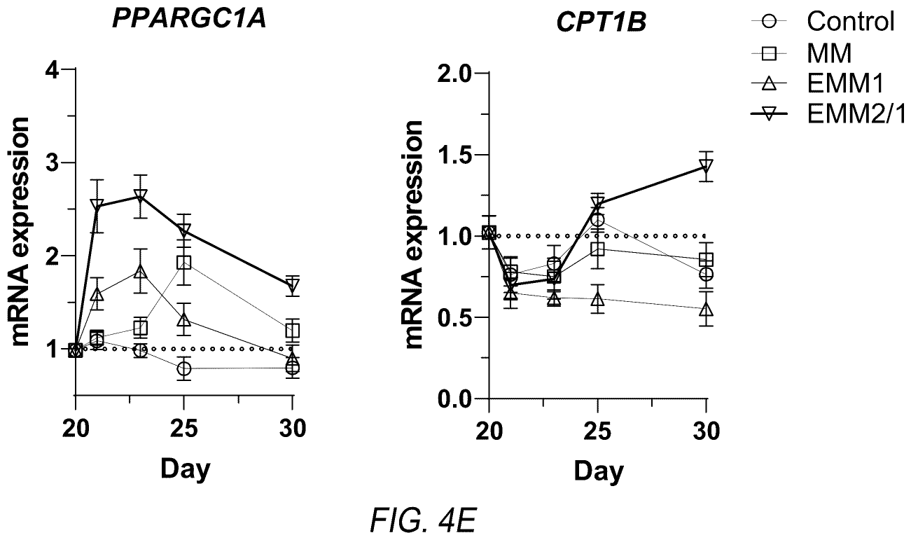

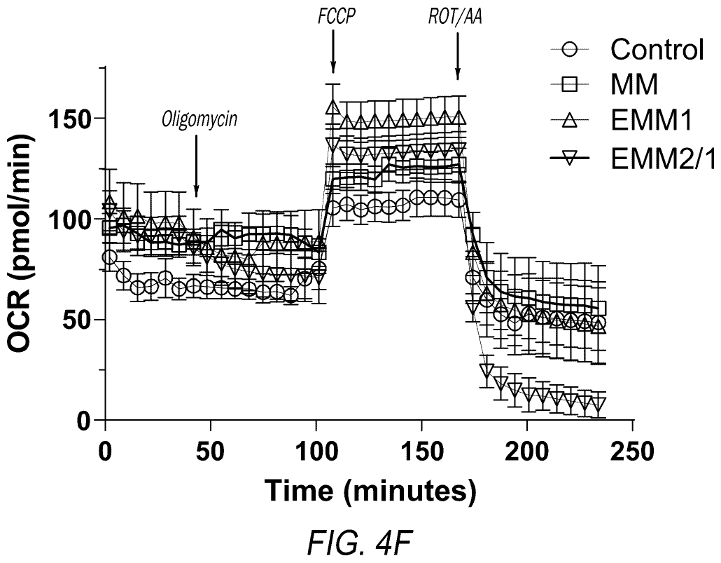

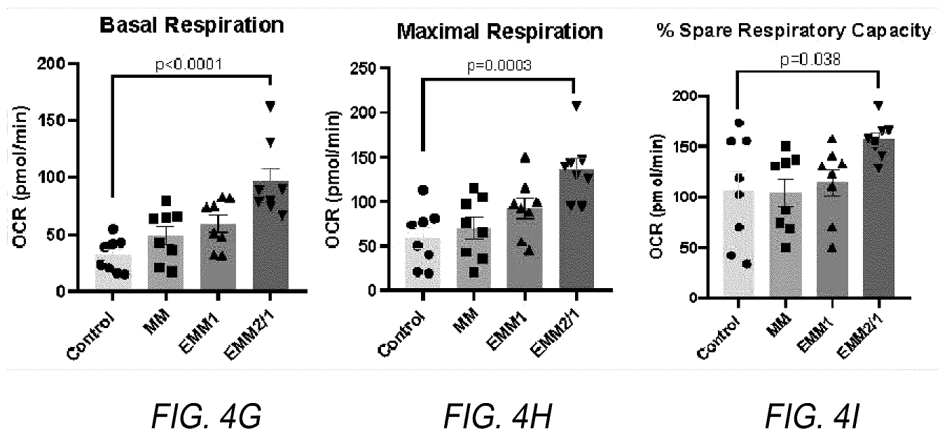

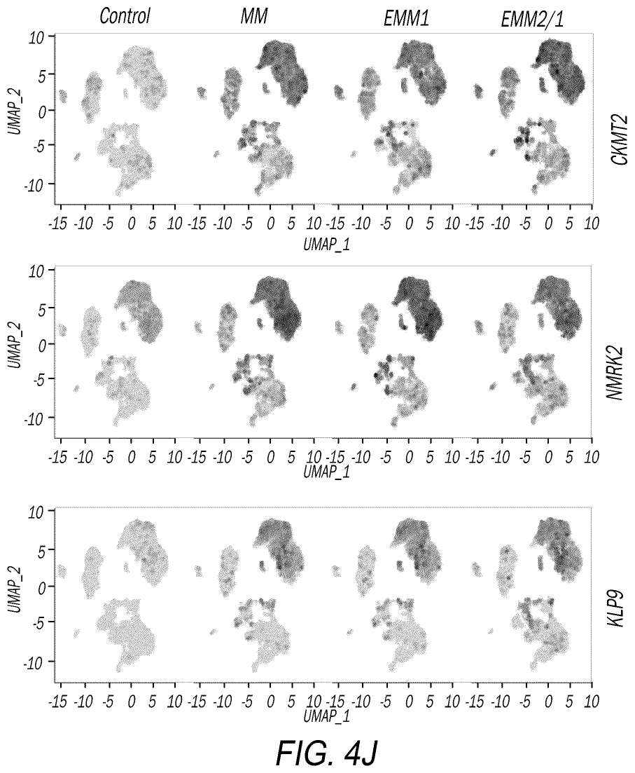

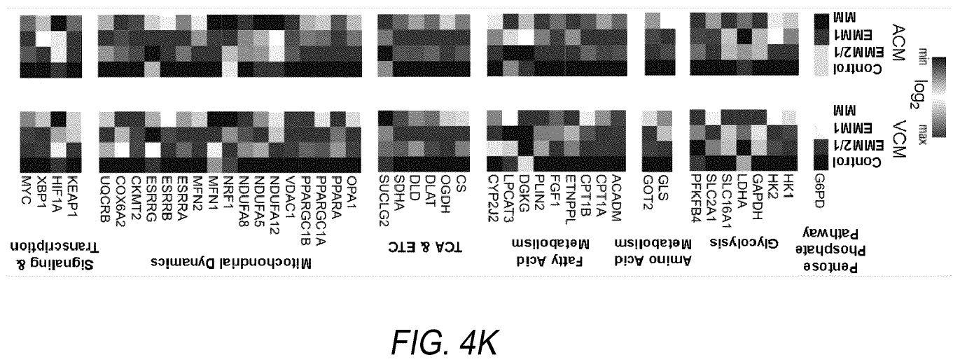

The early developing human heart relies heavily on glycolysis for energy expenditure. As it continues to grow, it decreases its reliance on glycolysis and switches to fatty acid oxidation for the bulk of energy consumption^{28,30,95-97}. Therefore, it was sought to determine the effect of the disclosed developmental induction conditions, and particularly EMM2/1, exerted on mitochondrial growth and metabolic transcriptional activity within heart organoids. Through the addition of MitoTracker, a mitochondrial-permeable fluorescent, live mitochondrial content within heart organoids at day 30 of culture was visualized (FIG. 4A). Control organoids displayed few and diffuse mitochondria, while EMM2/1 organoids possessed the most developed mitochondrial content of all conditions (abundance, morphology) (FIGS. 4A-4B). An increasing trend of mitochondrial content in MM, EMM1, and EMM2/1 organoids (fold change of 1.73±0.10, 2.60±0.11, and 3.10±0.18, respectively) was quantified relative to control, suggesting that developmentally maturated organoids had an increasingly higher capacity for aerobic respiration and responded positively to maturation stimuli (FIG. 4B). TEM revealed high-magnification detail on mitochondrial presence within organoids at day 30 of culture (FIG. 4C). Compared to day 15 mitochondrial size, Control organoid mitochondrial size was similar (FIG. 4D). However, mitochondrial size within MM, EMM1 and EMM2/1 organoids was dramatically increased relative to that of Control organoids. qRT-PCR was employed at different timepoints from day 20 to day 30 of organoid culture to explore the differential gene expression of two key OXPHOS genes in cardiac metabolic maturation: PPARGC1A, a master regulator of mitochondrial biogenesis⁹⁸, and CPT1B, a critical rate-limiting fatty acid transporter element^{99,100} (FIG. 4E). CPT1B expression increased 1.5-fold at day 30 in the EMM2/1 condition relative to control, yet expression in MM and EMM1 organoids decreased ~1-2-fold. PPARGC1A levels were up to 2.5-fold higher in EMM2/1 organoids from days 21 to 25 relative to Control and ended at a fold change of 1.5-fold higher by day 30. Expression for MM and EMM1 organoids also exhibited an increase from days 21 to 25 relative to Control, albeit not as high as EMM2/1. By day 30, expression in EMM1 organoids remained similar to Control while MM and EMM2/1 organoids displayed 1.2-fold and 1.7-fold higher levels, respectively. CPT1B expression increased 1.5-fold at day 30 in the EMM2/1 condition relative to Control, yet expression in MM remained similar or decreased for EMM1 organoids.

To investigate real-time metabolic parameters, Agilent Seahorse Mito Stress Test assays were performed with organoids in each condition (FIG. 4F). Organoids in the EMM2/1 condition displayed marked increases in basal respiration (FIG. 4G), maximal respiration (FIG. 4H), and percent spare respiratory capacity (FIG. 4I) compared to Control; aligning closely with the metabolic enhancement present in EMM2/1 organoid displayed in previous mitochondrial and metabolic data.

Supporting these findings using scRNAseq data, key genes involved in cardiac metabolism were found to be upregulated in organoids from the MM, EMM1, and EMM2/1 conditions (FIG. 4J), including: CKMT2, a gene that encodes a mitochondrial creatine kinase and is important for metabolic efficiency and implicated in cardiac maturation; NMRK2, involved in cardiac maturation and lipid metabolism, and is activated in high energy states; and KLF9, a gene related to adipogenesis and cardiac metabolic maturation. These genes were largely upregulated in the ACM and VCM clusters. Gene expression data from the ACM and VCM clusters was then used to look for a wider set of metabolic markers as the organoids developed in the different conditions. It was found that organoids in the EMM2/1 condition expressed much higher levels of key metabolic genes compared to Control, including those involved in fatty acid metabolism, amino acid metabolism, TCA cycle, and mitochondrial dynamics (FIG. 4K). Furthermore, computational transcriptomic analysis and mapping was performed to KEGG metabolic pathways using Pathview^{101,102} (data not shown). In agreement with the other metabolic data, EMM2/1 organoids showed reduced activity of glycolytic complexes (data not shown) and increased activity of mitochondrial respiratory complexes (data not shown), indicative of progressive developmental maturation. Overall, these results suggested that EMM2/1 organoids recapitulate significant aspects of cardiac metabolism in vitro reminiscent of fetal cardiac development at similar stages.

### Developmental Induction Conditions Promote Progressive Electrophysiological Maturation in Human Heart Organoids.

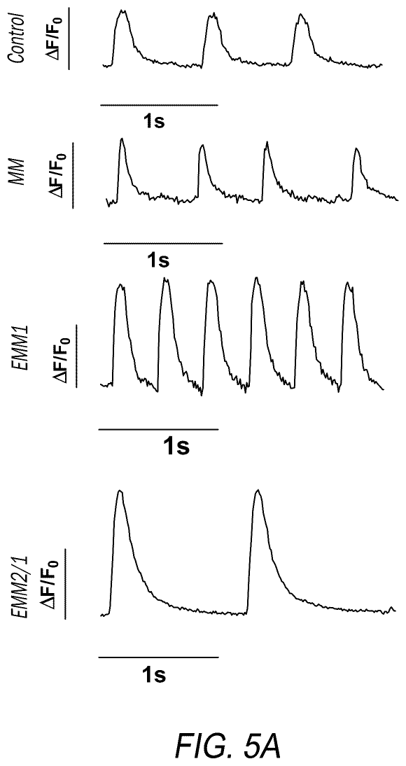

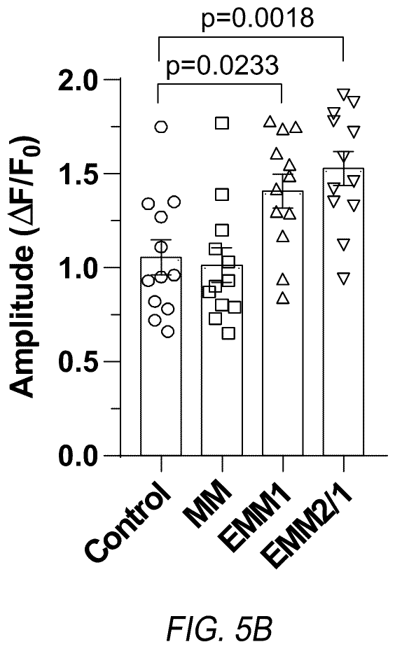

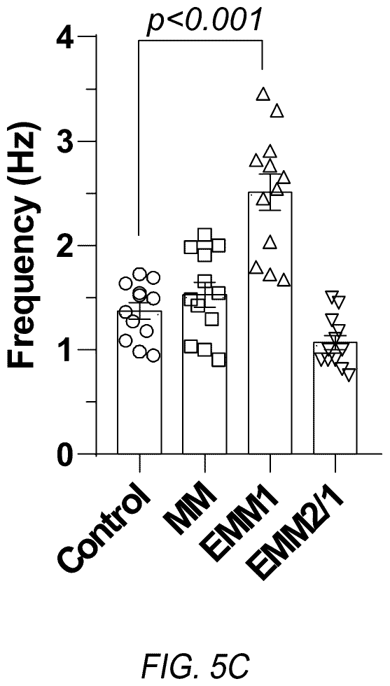

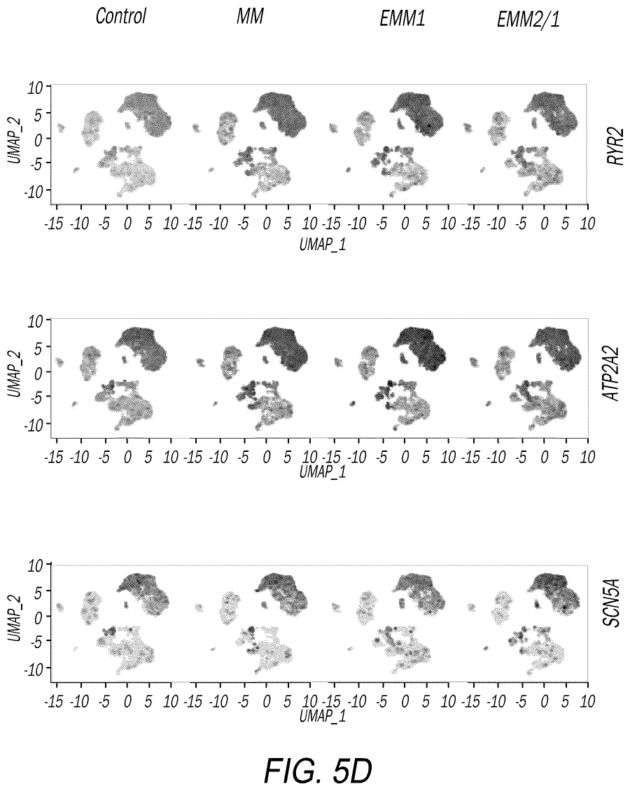

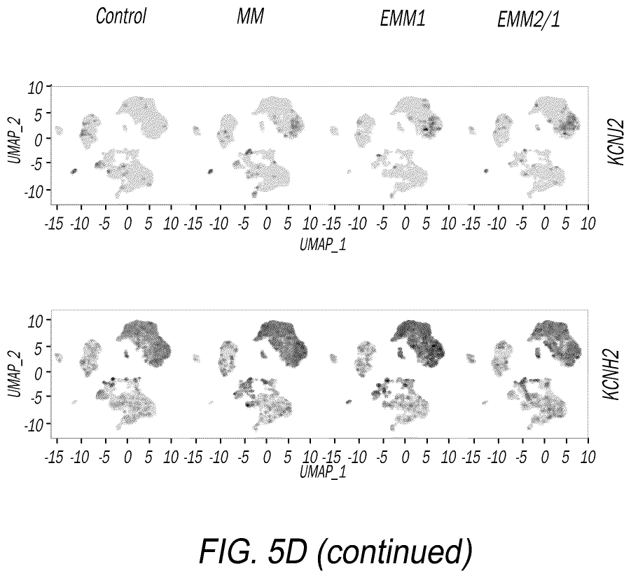

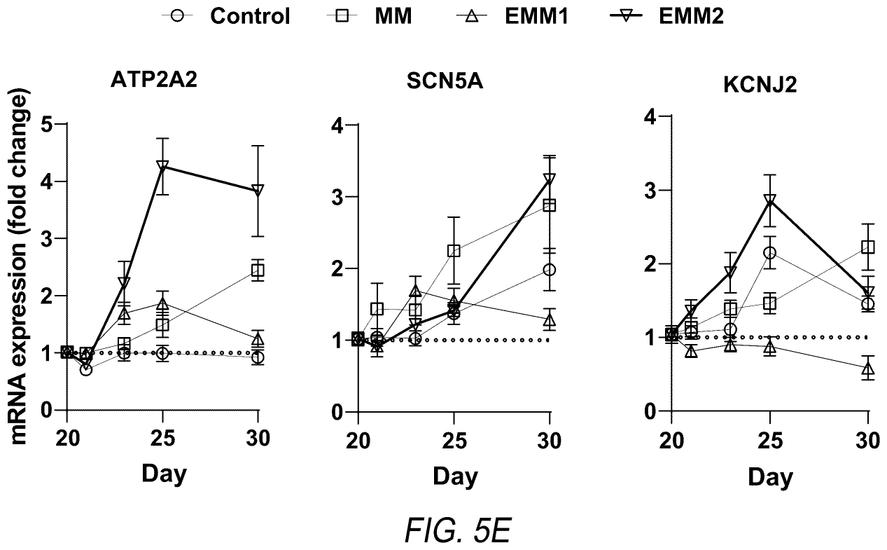

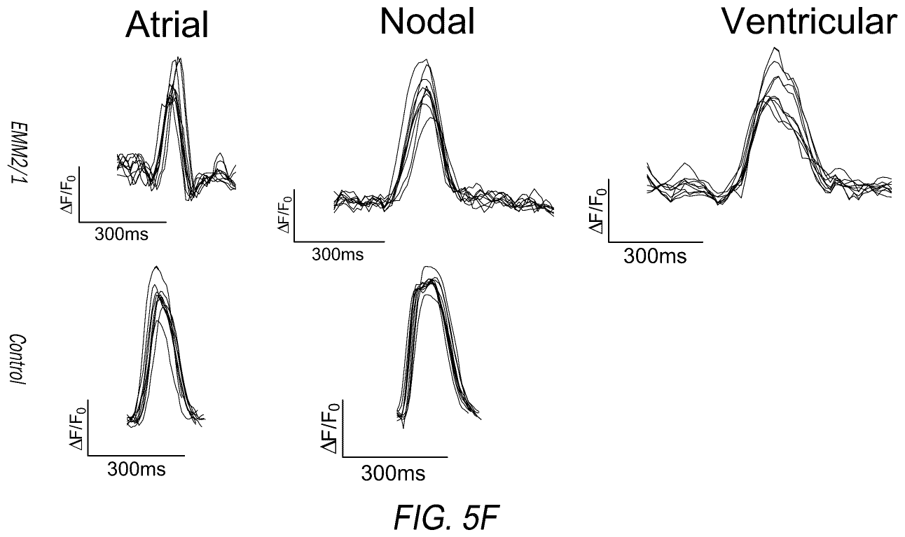

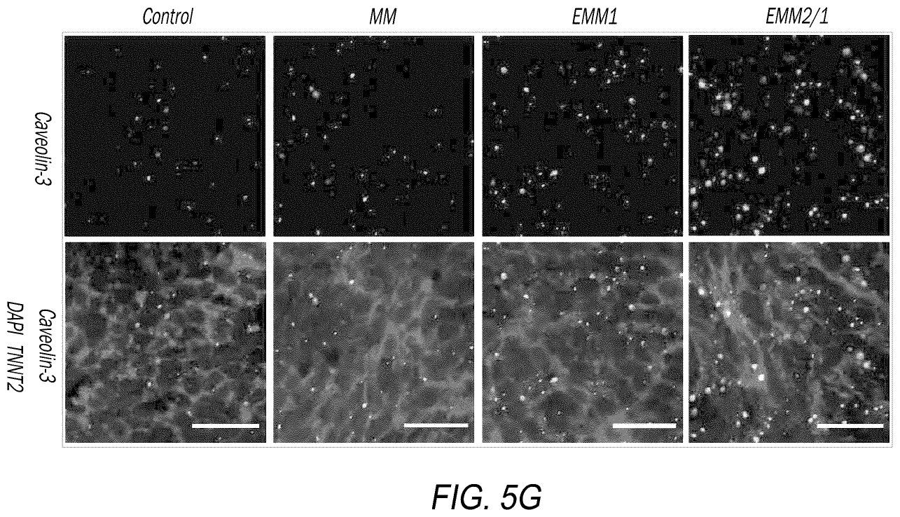

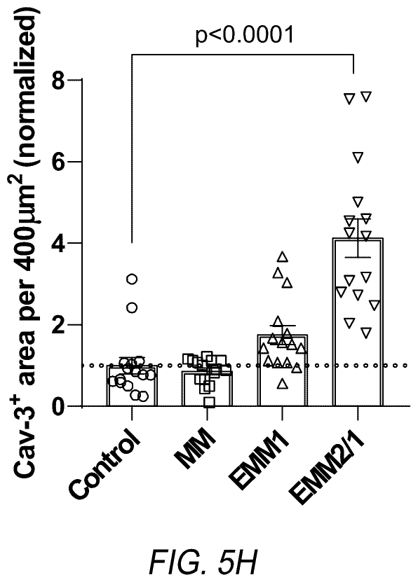

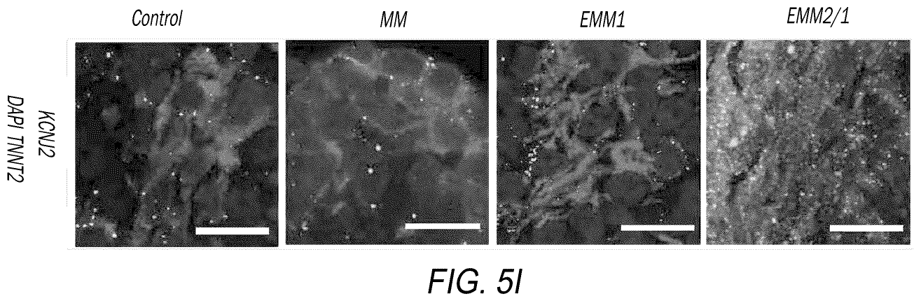

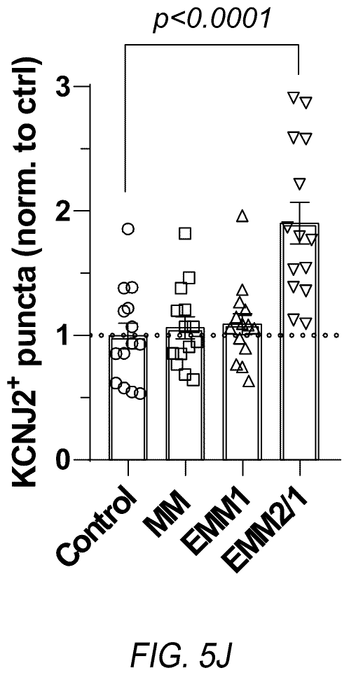

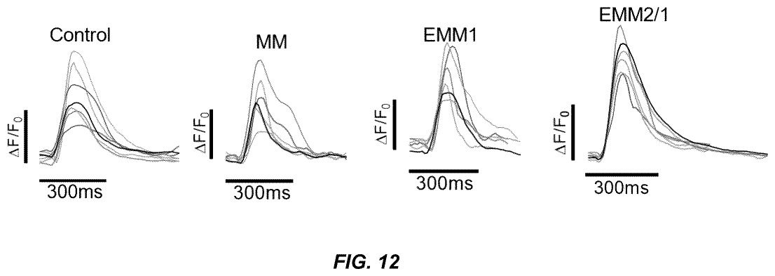

The emergence and presence of the cardiac conduction system, including specific ion channels and membrane receptors, such as those surrounding calcium, potassium, and sodium currents, represent critical elements of the cardiomyocyte action potential¹⁰³⁻¹⁰⁵ and fetal heart development^{106,107}. It was sought to characterize the functionality of heart organoids under developmental induction conditions through electrophysiology and immunofluorescence for key markers. Calcium transient activity of individual cardiomyocytes within human heart organoids at day 30 was assessed using the membrane-permeable dye Fluo-4 (FIG. 5A). Organoids in all conditions exhibited distinct and regular calcium transient activities with varying peak amplitude and action potential frequencies (FIGS. 5B-5C). Control and MM organoids presented smaller peak amplitudes when compared to EMM1 and EMM2/1 organoids, indicating less robust contractions (FIG. 5B), and also presented similar beat frequencies ~1.5 Hz. EMM1 organoids displayed abnormally high beating rates (~2.5 Hz) for the heart at this stage, while EMM2/1 organoids showed beat frequencies at ~1-1.5 Hz (FIG. 5C). In general, and except for EMM1 organoids, developmentally induced organoids presented beating rates compatible with what has been described for early human embryos at GD45^{108,109} (60-80 beats per minute). Calcium traces from organoids in all conditions were shown to be reproducible (FIG. 12A).

The total electrophysiological activity encompassing the cardiomyocyte action potential involves the complex orchestration of various ion currents, such as calcium, potassium and sodium, and supporting channels such as ryanodine receptors. The expression levels of various electrophysiological genes in heart organoids were investigated and a robust expression pattern in ACM and VCM clusters across all conditions was discovered (FIG. 5D), including RYR, ATP2A2, SCN5A, KCNJ2, and KCNH2. Expression levels for all genes appeared to increase slightly to moderately for the EMM2/1 condition relative to Control. Notably, KCNJ2 expression increased dramatically for all maturation conditions relative to Control, particularly in the EMM2/1 condition. An additional ion channel of critical importance is the hERG channel¹¹⁰⁻¹¹² encoded by the gene KCNH2. Mutations and perturbations in this channel can lead to shortening or prolongation of the QT intervall^{111,113-115}, and drug interactions with this channel can lead to cardiac arrythmia which represents a critical bottleneck surrounding drug discovery and development^{116,117}. KCNH2 expression within the organoids displayed high expression levels within the ACM and VCM clusters in all conditions.

Further, autonomic control of the cardiac conduction system through adrenergic signaling plays a large role in physiological functionality¹¹⁸⁻¹²⁰, and underlies a range of CVDs from heart failure and hypertension to arrythmia^{121,122}. The presence of critical beta-adrenergic receptor genes, ADRB1 and ADRB2, encoding beta-adrenergic receptors 1 and 2 was identified within the organoids in each condition (data not shown). While ADRB2 was expressed in both the ACM and VCM clusters in each condition, ADRB1 showed expression within the ACM and VCM clusters in MM, EMM1 and EMM2/1 conditions, but was only expressed in the ACM cluster in the Control condition. ADRB3 was sparsely expressed relative to ADRB1 and ADRB2, which stays true to cardiac physiology^{41,123-125}.

To investigate the temporal dynamics of key ion channels through the application of our developmental maturation strategies, qRT-PCR was utilized from day 20 to day 30 of organoid culture to assess levels of calcium (ATP2A2), sodium (SCN5A), and potassium (KCNJ2) transporters (FIG. 5E). ATP2A2 expression increased in all conditions relative to Control, with EMM2/1 exhibiting the most marked upregulation of 4-fold at day 25 and day 30. SCN5A expression was upregulated for all conditions from day 21 to day 30. Notably, MM and EMM2/1 organoids displayed a 3-fold increase at day 30 relative to Control organoids only displaying a 2-fold increase. KCNJ2 expression steadily decreased in the EMM1 condition relative to Control, with MM organoids exhibiting upregulation at day 30 and EMM2/1 displaying upregulation compared to Control throughout the culture period.

The voltage activity within Control and EMM2/1 heart organoids via the potentiometric dye di-8-ANEPPS was investigated and unique actional potentials within individual cardiomyocytes indicative of the presence of specialized atrial-, and nodal-like cells were identified but, interestingly, ventricular-like action potentials were only observed in EMM2/1 organoids (FIG. 5F).

Furthermore, proper excitation-contraction coupling, and depolarization and repolarization of cardiomyocytes depends on specialized invaginations of the sarcolemma (t-tubules), which are indicative of cardiomyocyte maturation^{126,127}. T-tubule presence was assessed in human heart organoids at day 30 via Caveolin-3 immunofluorescence imaging (FIG. 5G) and t-tubules were discovered among and surrounding sarcomeres (TNNT2+) within organoids in each condition, with increasing t-tubule density quantified in the EMM2/1 condition (FIG. 5H). Fluorescently labeled wheat germ agglutinin (WGA) was also utilized to assess t-tubules in human heart organoids at day 30 (data not shown) and discovered similar results indicating EMM2/1 organoids possessed marked increases in t-tubule density. The presence of the potassium ion channel KCNJ2 via confocal microscopy was also assessed (FIG. 5I). KCNJ2⁺ puncta were observed in each condition, with a 2-fold increased presence in the EMM2/1 condition relative to Control (FIG. 5J), supporting previous data displaying increased amounts of KCNJ2 transcripts in EMM2/1 organoids. To investigate the temporal dynamics of key ion channels through the application of the developmental maturation strategies, qRT-PCR was utilized from day 20 to day 30 of organoid culture to assess levels of calcium (ATP2A2), potassium (KCNJ2), and sodium (SCN5A) transporters (FIG. 11A). ATP2A2 expression increased in all conditions relative to control, with EMM2/1 exhibiting the most marked upregulation of 7-fold. KCNJ2 expression steadily decreased in the EMM1 condition relative to control, with MM organoids exhibiting upregulation at day 30 and EMM2/1 displaying upregulation throughout the culture period. SCN5A expression showed upregulation for the MM condition at day 25 and 30, while EMM2/1 also displayed upregulation at day 30 relative to control. EMM1 expression remained consistent with control until day 25 and was downregulated at day 30. Together, this data shows that the developmentally maturated organoid platform, specifically the EMM2/1 strategy, produces organoids that recapitulate significant electrophysiological aspects of cardiac development, physiology, and disease.

### Developmental Induction Promotes the Emergence of a Proepicardial Organ and Formation of Atrial and Ventricular Chambers by Self-Organization.

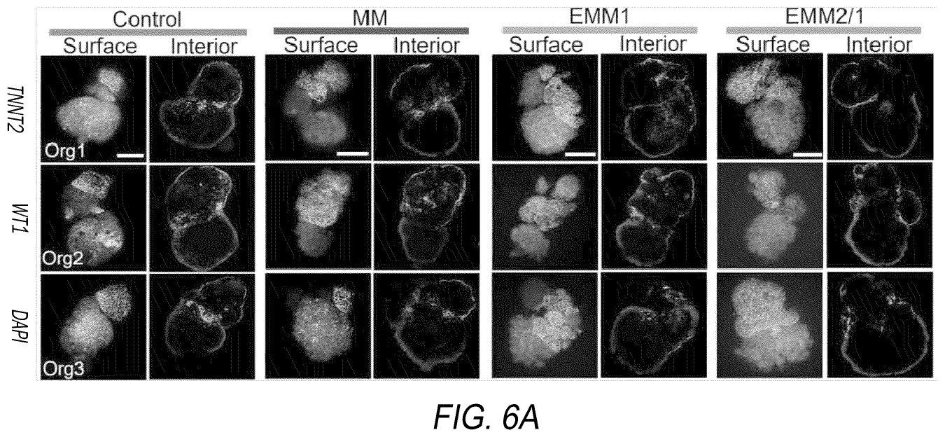

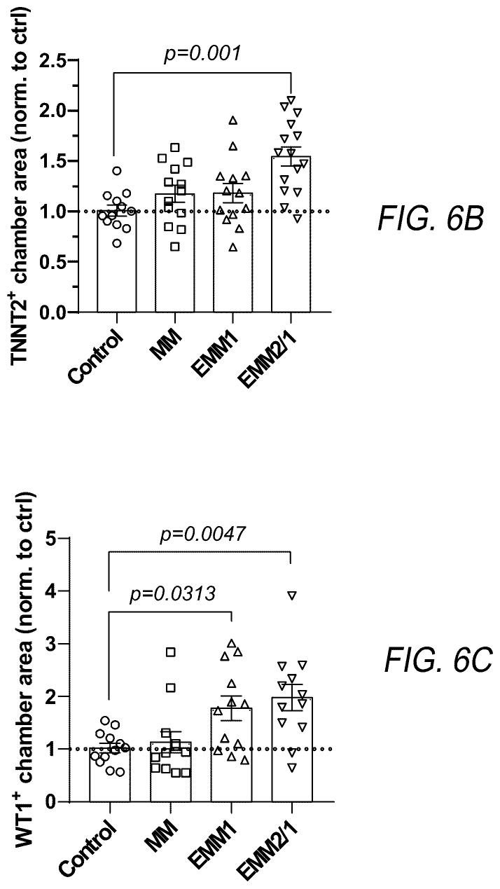

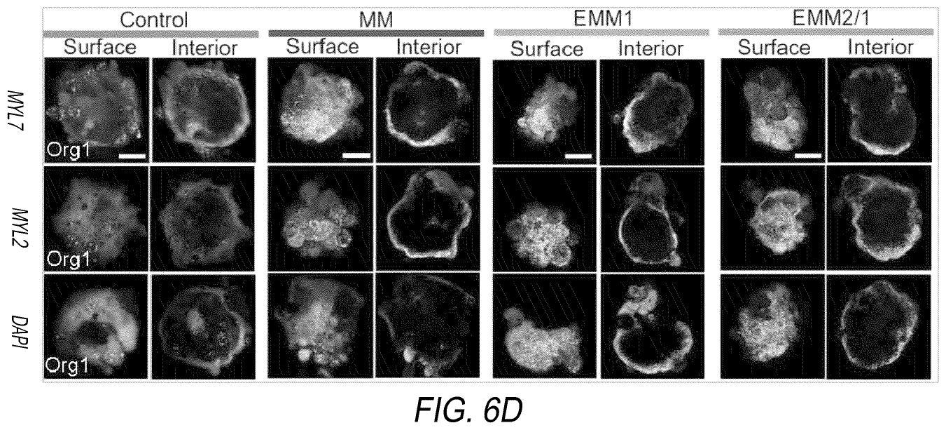

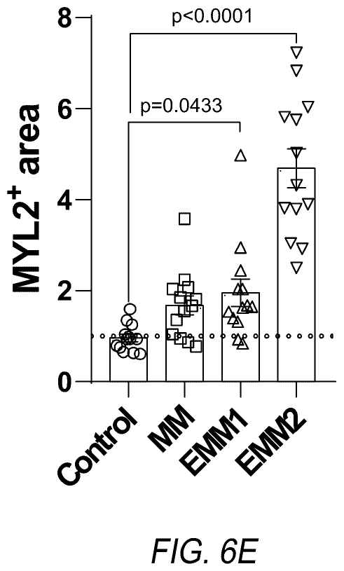

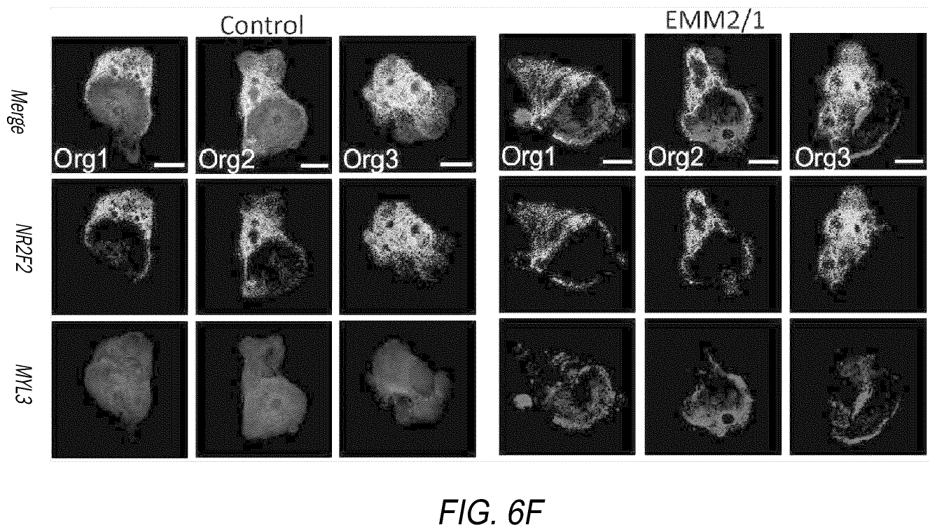

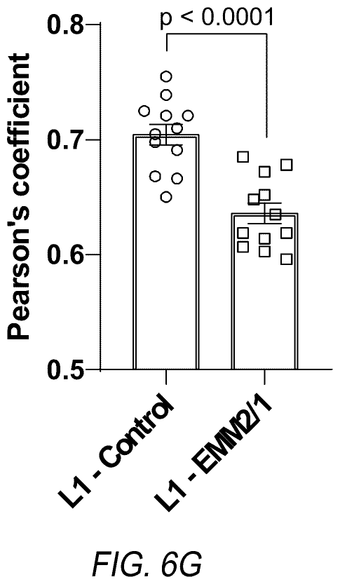

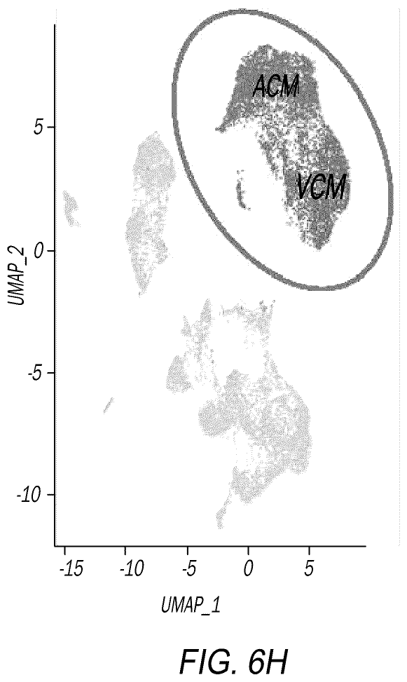

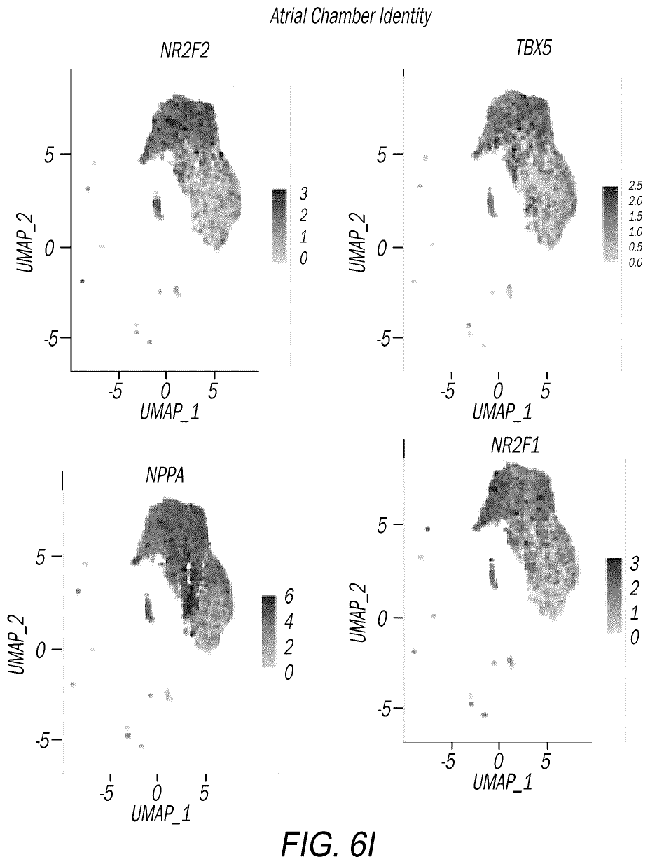

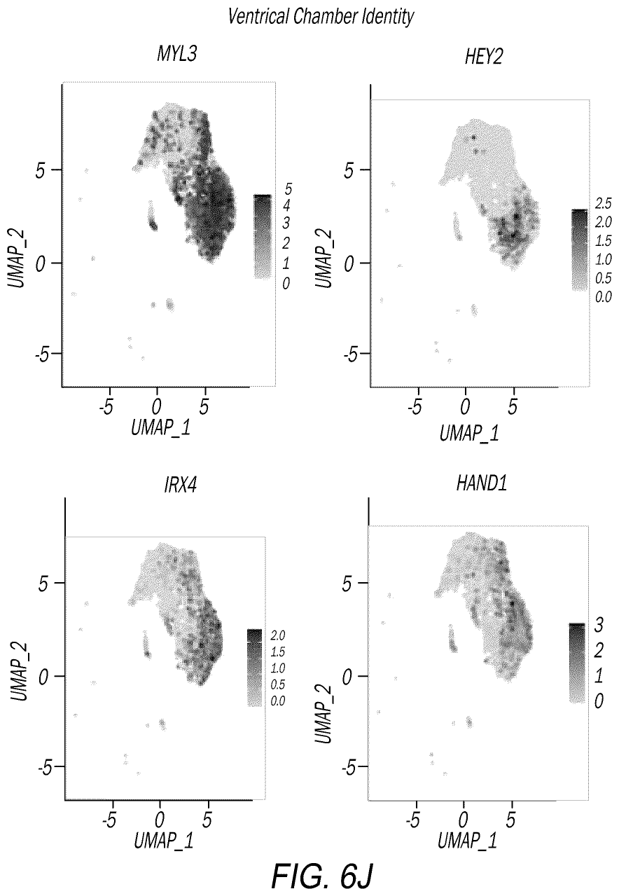

It was shown that developmentally-induced heart organoids presented improved cellular, biochemical and functional properties when compared to their control counterparts, and exhibited multiple features reminiscent of GD45 human fetal hearts. However previous heart organoid attempts have been lacking in producing anatomically relevant cardiac structure and morphology to a great extent, including previous work¹⁵⁻¹⁷ Given the significant changes observed through applying the EMM2/1 strategy, it was decided to characterize morphological changes that took place under this improved conditions. Organoids were harvested on day 30 of culture and stained for WT1 (proepicardium and epicardial cells) and TNNT2 (cardiomyocytes) (FIG. 6A). Organoids in each developmental induction condition displayed TNNT2⁺ and WT1⁺ cells, consistent with previous observations¹⁵, indicating the presence of epicardial and cardiomyocyte populations widely distributed through the organoids. Assessing both surface and interior planes of the organoids, organoids in all conditions were observed to possess two distinct “chambers” marked via WT1⁺ and TNNT2⁺ cells. TNNT2⁺ cells were densely packed and formed a thick myocardial wall in the lower chamber, while also present in the upper region in a less dense arrangement directly underneath WT1⁺ cells. In EMM2/1, WT1⁺ cells were found densely covering the outer surface of the budding region, while existing in scattered, distant populations on the surface of the lower region. These staining patterns were not observed in the control, MM or EMM1 culture conditions. The area of WT1⁺ and TNNT2⁺ chambers was quantified across all maturation conditions (FIGS. 6B-6C). No difference was found in TNNT2⁺ chamber area in MM and EMM1 organoids relative to Control but it was found that EMM2/1 organoids display an increased area (fold change) of 1.54 relative to Control. Additionally, no difference was found in WT1⁺ chamber area in MM organoids relative to Control, whereas EMM1 and EMM2/1 organoids displayed increased areas (fold change) of 1.77 and 1.98, respectively. This data shows that organoids in all conditions undergo significant morphological changes that lead to highly specific and reproducible cellular organization, including the emergence of an organoid with advanced myocardial dual-chamber morphology as well as a proepicardial pole.

Upon further examination, it could be determined that ventricular (MYL2) and atrial (MYL7) myosins, indicative of ventricular- and atrial cardiomyocyte subpopulations respectively, were spatially restricted to a great extent, particularly in EMM2/1 organoids. (FIG. 6D). All organoids expressed MYL7 throughout the bulk of the organoid, but expression was stronger in the upper chamber in EMM2/1, suggesting an atrial-like chamber. In the Control and MM conditions, organoids possessed MYL2 in a high variety of locations that were not restricted to a polar end of the organoid or to either chamber in particular. On the other hand, organoids in the EMM1 and EMM2/1 conditions displayed an increasing prominence of MYL2⁺ staining and degree of organization, showcasing MYL2 restricted to one polar end of the organoids and with EMM2/1 organoids displaying a 5.5-fold increase in MYL2⁺ area (FIG. 6E); suggesting the formation of a ventricular-like chamber. These findings were particularly interesting because in these EMM2/1 organoids the proepicardial region was directly located over the atrial chamber, with the ventricular chamber in the opposite side of the organoid. Overall, this organization is comparable to the anterior-posterior patterning axis present in in utero in the forming heart tube (see FIG. 7A for a schematic).

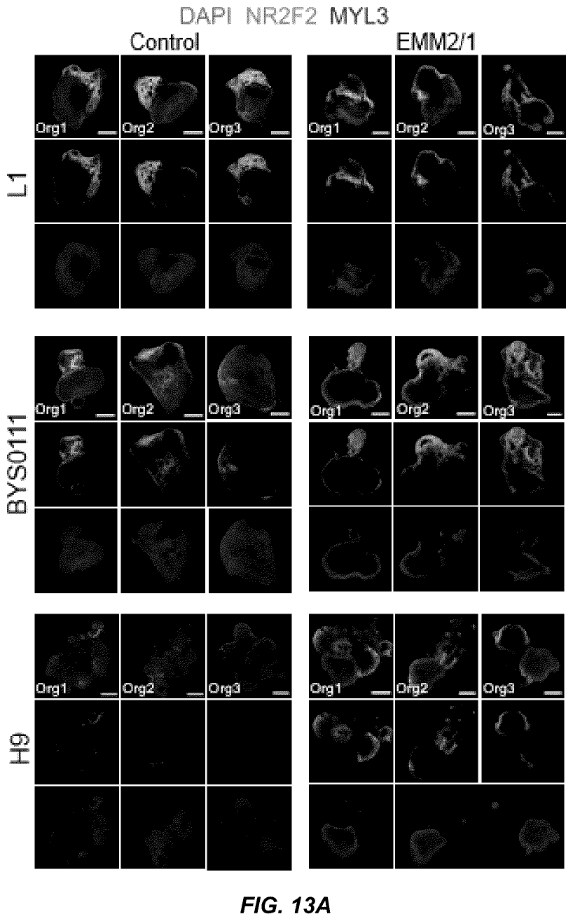

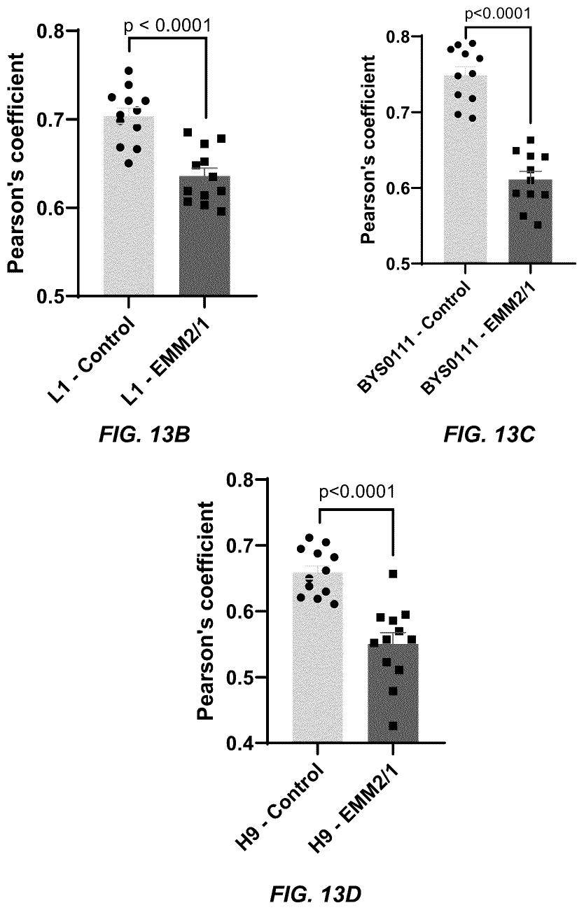

To further investigate the identity of ventricular- and atrial-like chambers in the human heart organoids, staining for additional atrial and ventricular chamber markers NR2F2 (atrial) and MYL3 (ventricular) (FIG. 6F) was performed. EMM2/1 organoids displayed a distinct, increased degree of separation between the two chambers, while Control organoids showed a larger overlap of these two proteins (FIG. 6G); indicating that EMM2/1 organoids possess a greater degree of specification and maturity of chamber development. Remarkably, these results were reproduced in two additional PSC lines: BYS0111 (iPSC) and H9 (ESC) (FIG. 13A). While L1 Control and EMM2/1 organoids were showed again for reproducibility and comparative purposes (FIG. 13B), Control BYS0111 organoids displayed similar overlap of NR2F2 and MYL3, while EMM2/1 BYS0111 organoids showed distinct separation of NR2F2 and MYL3 (FIGS. 13A and 13C), with MYL3⁺ cells highlighting thick myocardial walls in the EMM2/1 condition. Control H9 organoids displayed marked decreased expression of both NR2F2 and MYL3 compared to EMM2/1 H9 organoids, with EMM2/1 H9 organoids displaying distinct separation of NR2F2⁺ and MYL3⁺ chambers (FIGS. 13A and 13D). To support these immunofluorescence results describing the potential identity of atrial and ventricular chambers in our heart organoids, gene expression patterns using scRNAseq data in the ACM and VCM clusters (FIGS. 6H-6J) investigated. ACMs displayed increased gene expression for hallmark atrial chamber identity markers such as NR2F2, TBX5, NPPA, and NR2F1 (sources) compared to VCMs (FIG. 6I). Meanwhile, VCMs showcased increased gene expression for hallmark ventricular chamber identity markers such as MYL3, HEY2, IRX4, and HAND1 (sources) compared to ACMs (FIG. 6J). These results highlight not only the power of the present heart organoid platform to recapitulate significant structural events in heart development, but also the ability of the EMM2/1 strategy to elicit distinct cell types, morphological profiles, and chamber identity across multiple PSC lines.

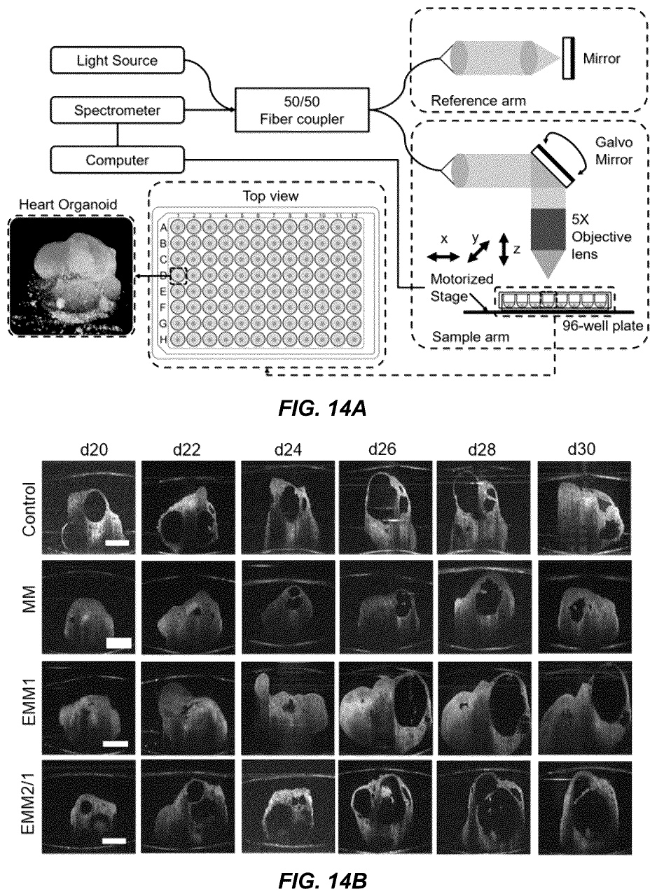

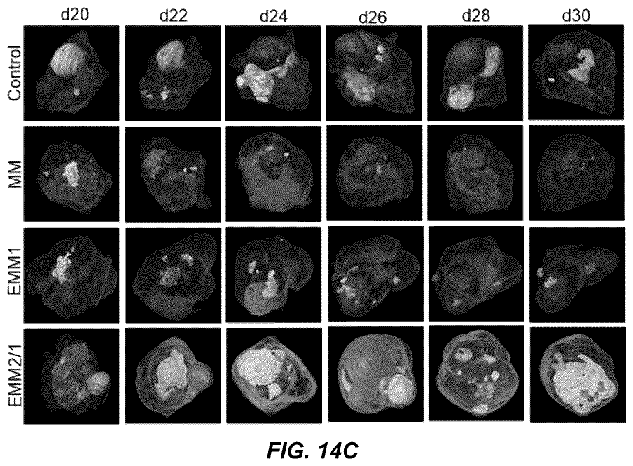

To continue an investigation into heart organoid chamber morphology and profiles, optical coherence tomography (OCT) was used to live image organoids over time, and to measure the growth and monitor dynamics of chamber development under developmental induction conditions via a custom-made OCT microscopy system amenable to high-content screening^{128,129} (FIG. 14A). It was found that chambers exhibited a highly dynamic behavior initially and coalesced into larger structures as time passed. EMM2/1 conditions led to the largest internal chambers within the present human heart organoids between day 20 and day 30 of culture, with typically two large internal chambers as previously observed by confocal microscopy (FIG. 14B-14C). While MM organoids displayed a single internal chamber, organoids grown in the control, EMM1 and EMM2/1 conditions possessed multiple, smaller, interconnected chambers. Control and EMM2/1 organoids possessed chambers throughout the bulk of the organoids while EMM1 organoids showed chambers predominantly towards one side of the organoid. These data confirmed the formation of well-established cardiac chambers and further supported the observations on the effects of developmental induction conditions.

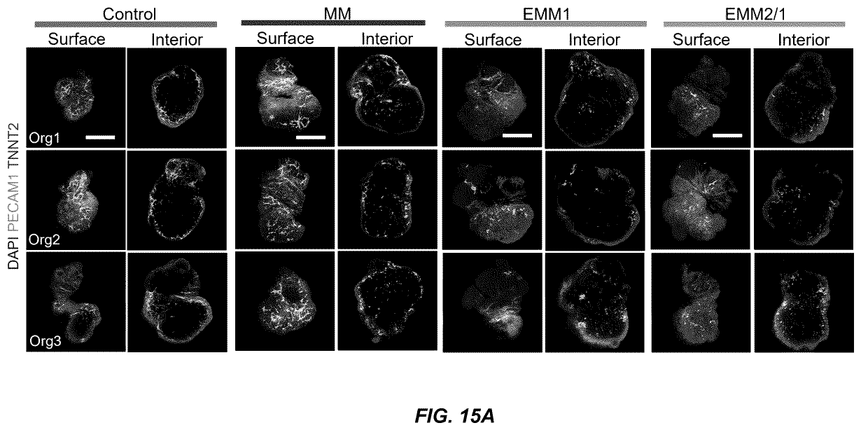

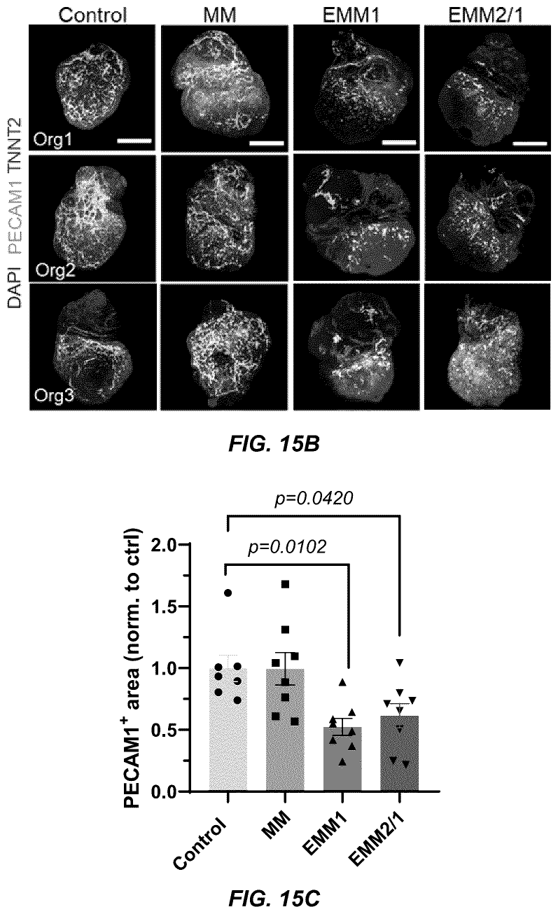

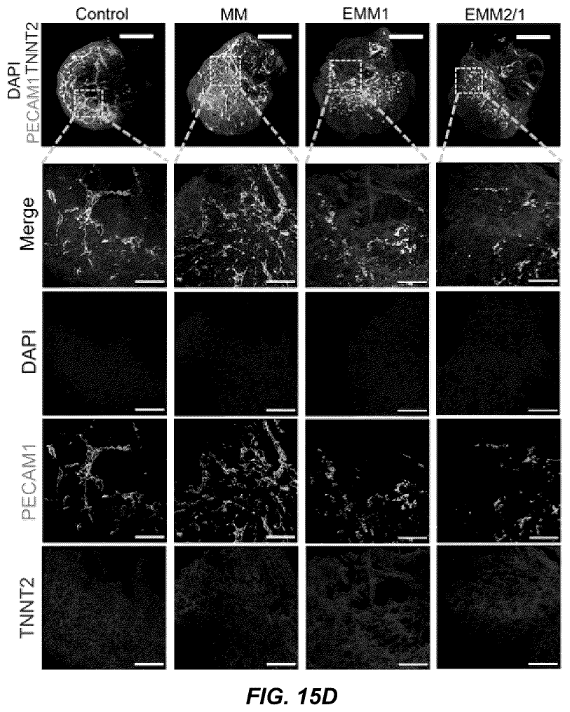

Vasculature formation in developmental induction conditions was also assessed. Endothelial cell (PECAM1+) vasculature formation was examined at day 30 of culture via immunofluorescence and confocal microscopy (FIGS. 15A-15D). Assessment of organoids on surface and interior planes revealed the presence of endothelial cells amongst the myocardial regions of all organoids (FIG. 15A). Organoids in the EMM1 and EMM2/1 conditions presented less PECAM1+ cells than control and MM organoids. Control and MM organoids displayed robust, interconnected endothelial cell networks and throughout myocardial (TNNT2+) tissue (FIG. 15B). Total PECAM1+ area was quantified, and MM organoids showed no significant difference compared to control organoids, while EMM1 and EMM2/1 organoids possessed only 52% and 61% of PECAM1+ area compared to controls, respectively (FIG. 15C). High magnification images of organoids further show the morphological transitory state of the endothelial cells within cardiomyocyte-rich regions (FIG. 15B). Overall, this data suggests that vascularization of the organoids might be partially trumped by factors in EMM1 and EMM2/1 conditions, possibly due to timing or concentration of growth factors, and will require further investigation to fine tune medium conditions.

### An Endogenous Retinoic Acid Gradient is Responsible for the Spontaneous Anterior-Posterior Heart Tube Patterning in EMM2/1 Organoids.

The emergence of a spatially restricted retinoic acid gradient originating at the posterior pole of the heart tube (produced by the epicardium and primitive atrium) is a critical developmental step in heart development in mammals. This gradient establishes the anterior-posterior axis that provides cues for the formation of the ventricles and the inflow and outflow tract, while also contributing to the specification of cardiogenic progenitors, and possible other structures^{130,131} (FIG. 7A). To determine whether the heart tube-like structure observed in EMM2/1 conditions (FIG. 6) was indeed reminiscent of retinoic acid-mediated heart patterning, Raman microscopy was performed to detect its molecular signature using a microscope designed for this purpose (data not shown). The presence of myosin, troponin T, tropomyosin, collagen I and other related molecular signatures were identified in organoids in all conditions as expected, and the presence of retinoic acid specifically in EMM2/1 (FIG. 7B). Retinoic acid synthesis is carried out largely by retinaldehyde dehydrogenase 2 (ALDH1A2) during embryogenesis¹³¹⁻¹³³. qRT-PCR was utilized from at day 30 of organoid culture to assess levels of ALDH1A2 in all conditions (FIG. 7C). ALDH1A2 expression was shown to increase by ~2.2-fold in the EMM2/1 condition relative to Control, with no significant changes in expression displayed in the MM or EMM1 conditions.

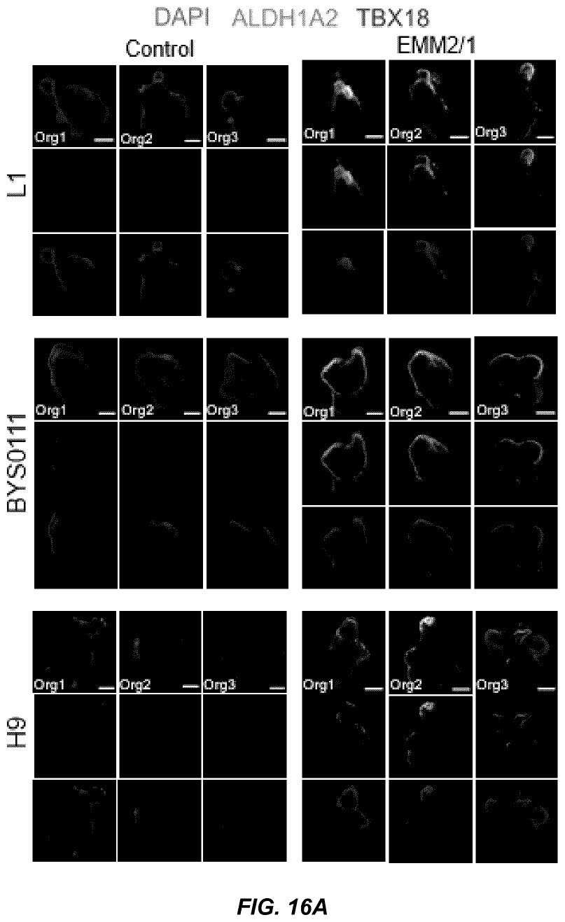

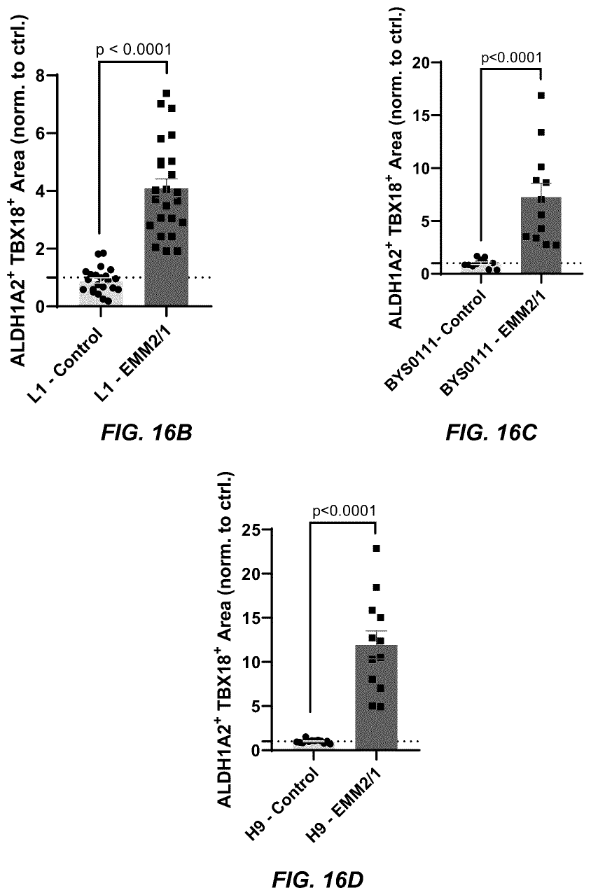

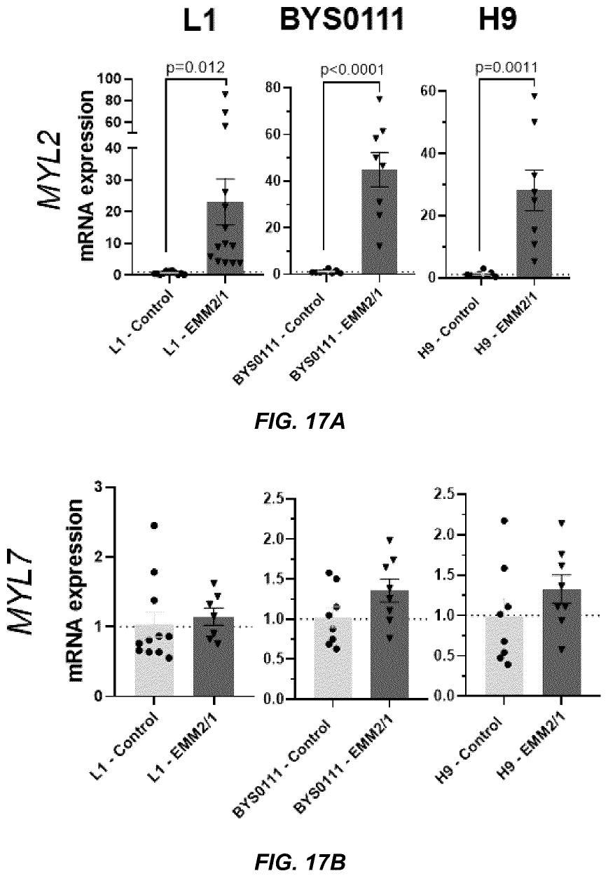

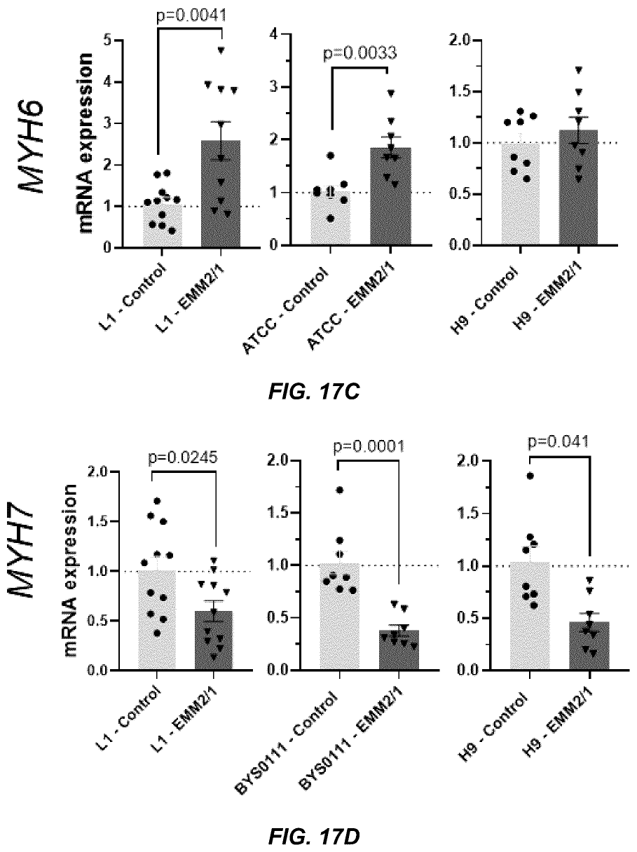

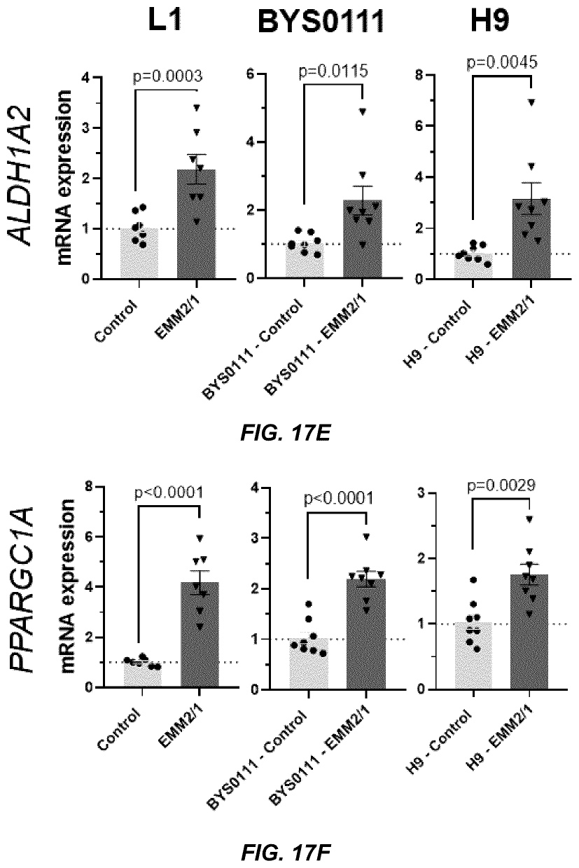

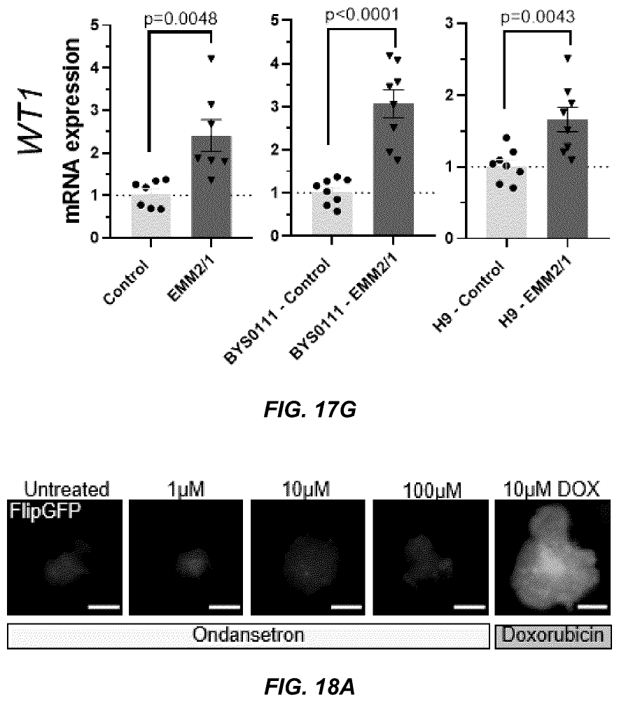

To assess the cell-specific dynamics of retinoic acid production in the organoids, scRNAseq data was used to show that ALDH1A2 is expressed in EPCs, PEDCs, and ACMs in the organoids (FIG. 7D); consistent with expression patterns reported in vivo. To complement this analysis and to further investigate the localization of retinoic acid production in the organoid, immunostaining with antibodies was performed for ALDH1A2 and for TBX18 (an epicardial transcription factor, to label the proepicardial organ/atrial pole)^{56,134,135} for organoids in all conditions at day 30. It was found that organoids in the EMM2/1 condition possess a localized, polarized expression of ALDH1A2 which colocalizes with TBX18⁺ cells, confirming that the retinoic acid gradient patterning the organoids was coming from the proepicardial/atrial pole (posterior pole of the heart tube in utero) (FIGS. 7A, 7E, and 7F). Control, MM and EMM1 organoids did not display ALDH1A2 expression. The area of colocalization between ALDH1A2 and TBX18 was quantified and showed that organoids in the EMM2/1 condition are significantly more responsive to the induction of retinoic acid synthesis (FIG. 7G). Remarkably, these results were reproduced in the two additional PSC lines, BYS0111 and H9 (FIG. 16A). While L1 Control and EMM2/1 organoids were displayed once again for reproducibility and comparative purposes (FIG. 16B), Control and EMM2/1 BYS0111 organoids displayed similar patterns of ALDH1A2 and TBX18 expression as was shown for L1 organoids; EMM2/1 BYS0111 organoids displayed markedly increased amounts of polarized ALDH1A2⁺TBX18⁺ cells compared to Control BYS0111 organoids (FIGS. 16A and 16C). H9 organoids displayed a similar degree of recapitulation, with EMM2/1 H9 organoids showcasing marked increases in ALDH1A2⁺TBX18⁺ cells compared to Control H9 organoids (FIGS. 16A and 16D). Control and EMM2/1 organoids from all three cell lines also displayed similarly robust and reproducible transcriptomes (FIGS. 17A-17G) for ALDH1A2 and other important genes such as MYL2, MYL7, WT1, and PPARGC1A, as determined by qRT-PCR.

To more robustly prove that retinoic acid contributes to patterning in EMM2/1 organoids, ALDH1A2 was inhibited using DEAB (sources), and through immunostaining for NR2F2 and MYL3, it was shown that ALDH1A2 inhibition leads to diminished heart organoid patterning (FIGS. 7H-7J). Organoids with inhibited ALDH1A2 displayed 0.35-fold and 0.42-fold reductions in MYL3⁺ and NR2F2⁺ areas relative to Untreated organoids, respectively. It was also shown that addition of exogenous retinoic acid does not lead to differences in patterning, suggesting that EMM2/1 organoids are already endogenously producing optimal amounts of retinoic acid.

Together, this data shows the ability of EMM2/1 organoids to endogenously synthesize retinoic acid in a spatially restricted manner colocalized with the epicardium (TBX18), a phenomenon that closely mimics the processes observed in in utero heart development and heart tube patterning.

### Ondansetron Treatment During Heart Organoid Development Captures Congenital Heart Disease Phenotypes.

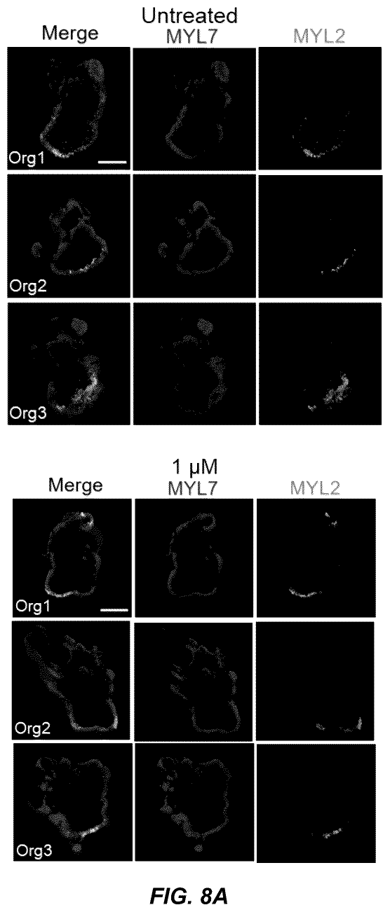

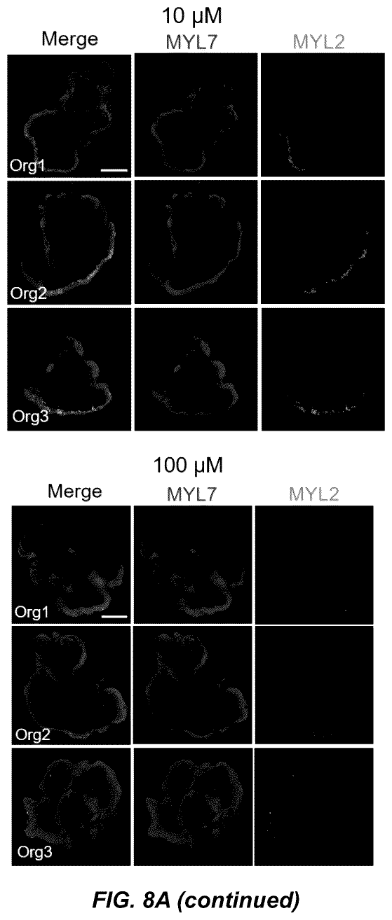

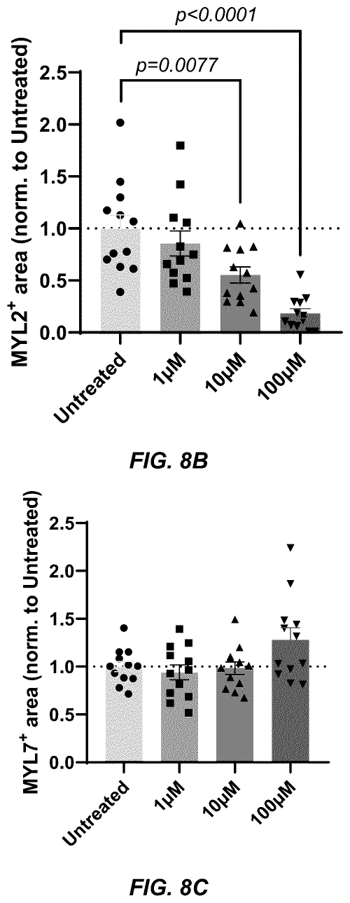

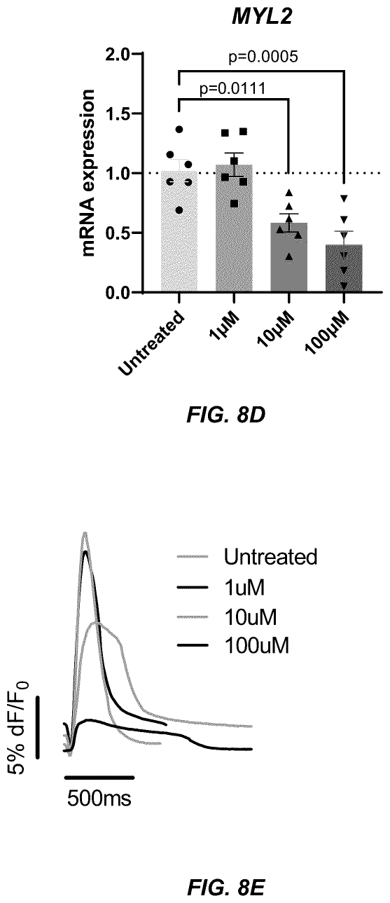

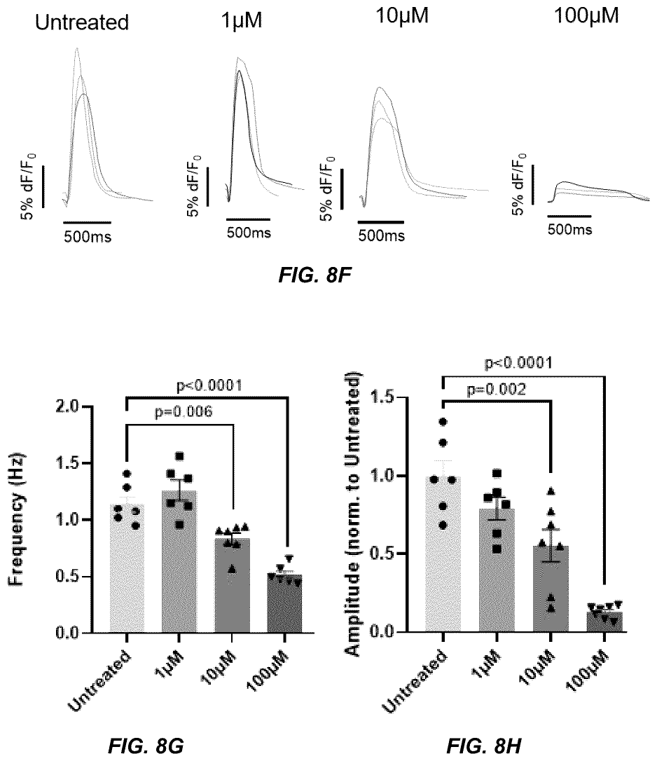

Organoids possess the unique capacity to better model and investigate human development, organogenesis and disease modeling at an unprecedented scale and precision. However, in the contexts of organogenesis and disease modeling, until now, human heart organoids have only been used to model developmental perturbations in diabetes-induced cardiomyopathy during pregnancy (Yoni), gene knockout studies (Drakhlis), developmental cryoinjuries (Hofbaur), and hypertrophic and fibrotic remodeling (Meier Epicardioid). Therefore, while heart organoids show promise towards unraveling unanswered questions surrounding cardiac organogenesis and pathology, critical areas such as investigating developmental drug toxicity and broader morphological perturbations in cardiopathologies remain ripe for discovery. During pregnancy, women are routinely prescribed the anti-emetic Ondansetron, also known as Zofran, for off-label use, with X % of pregnant women reported to take Ondansetron at some point during pregnancy. Nonetheless, Ondansetron has been implicated in causing congenital heart defects and orofacial defects, although the consensus in the field is divided and well-designed studies to investigate its safety are largely lacking. The difficulty and unethical nature of studying human heart development and human congenital heart defects represents a critical bottleneck surrounding the investigation of many facets of cardiac research. In this way, it was sought to investigate the effects of ondansetron during human heart organoid development (FIGS. 8A-8J). Clinical data was used on ondansetron plasma blood levels to determine relevant concentrations for the studies¹³⁶. Ondansetron was applied at three concentrations to heart organoids from day 9 until day 30 and applied the EMM2/1 strategy, then their morphology for MYL7 and MYL2 was assessed (FIG. 8A), two critical myosin proteins heavily involved in heart development (source), at day 30. Untreated organoids displayed the previously-shown morphology and patterning for MYL7 staining throughout the whole organoid and for MYL2 staining localized to one end of the organoid, reminiscent of a ventricular-like chamber. MYL2 is also a protein involved in ventricular septal defects, the principal heart defect implicated with Ondansetron use. Strikingly, as ondansetron concentration was increased, MYL2⁺ cells began to diminish, particularly in the 10 μM and 100 μM conditions (FIG. 8A). These results were quantified and showed that MYL2⁺ area decreased to 0.55-fold and 0.18-fold relative to Untreated (FIG. 8B), while MYL7⁺ area remained unchanged across all conditions (FIG. 8C). Organoids in the 100 μM condition also appeared to be structurally less organized with less defined chamber walls and loose chamber separation compared to Untreated. To support these results, qRT-PCR was performed on organoids in all conditions and showed that MYL2 expression decreases to 0.58-fold and 0.40-fold in the 10 and 100 μM conditions, respectively, relative to Untreated (FIG. 8D). Together, this data suggests that ondansetron may disturb critical steps of heart development through the repression of MYL2 at both a protein and transcriptional level.

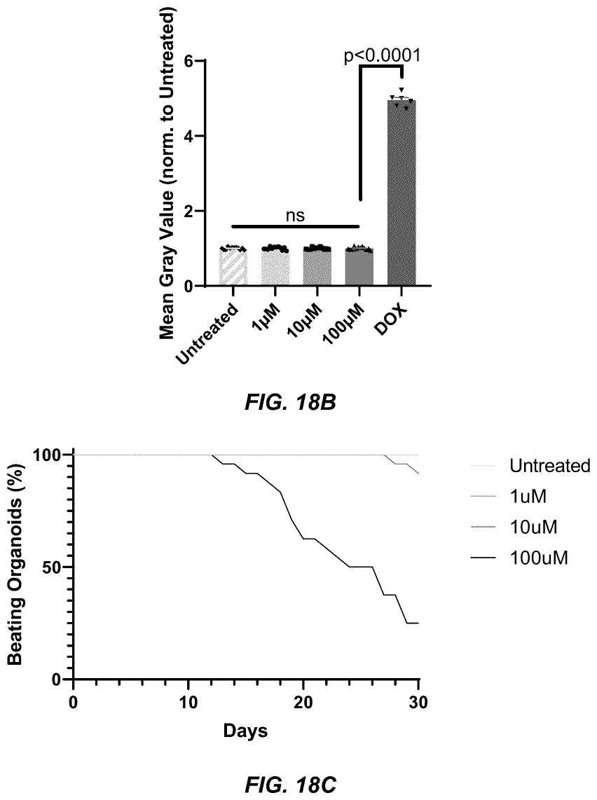

Ondansetron has been implicated in prolonging the QT interval, a potentially deadly phenomenon. The electrophysiological effects of ondansetron on heart organoid development were also investigated (FIGS. 8E-8J) via the potentiometric dye di-8-ANEPPS. Action potentials for organoids in the 10 and 100 μM conditions were markedly different compared to Untreated (FIGS. 8E-8F), displaying decreased frequencies FIG. 8G), amplitudes (FIG. 8H), and increased APD30/90 (FIGS. 8I-8J), suggesting that ondansetron elicits a progressive electrophysiological pathological phenotype during heart development. Interestingly, however, it was shown that, while ondansetron does not contribute to apoptosis in human heart organoids (FIG. 18A-18B), over time, ondansetron contributes to progressive loss of beating in heart organoids with the 100 μM condition exhibiting the most marked loss in cardiac activity (FIG. 18C).

Collectively, this data provides a first-of-its-kind insight into the morphological and electrophysiological effects of ondansetron during human development and provides a framework for future investigations towards safety and efficacy of gestational medications and pathology of congenital heart diseases.

All publications, patent applications, issued patents and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### Further Embodiments

Embodiment 1. A maturation medium comprising: a cell growth medium containing a medium supplement, wherein the medium supplement comprises one or more fatty acid, triiodothyronine (T3) growth hormone, insulin, one or more antioxidant, a sugar, and carnitine; one or more additional fatty acid; an additional carnitine or creatine; and an additional T3 growth hormone.

Embodiment 2. The maturation medium of embodiment 1, wherein the cell growth medium comprises Roswell Park Memorial Institute (RPMI) medium; Dulbecco's Modified Eagle's Medium (DMEM); a derivative of DMEM such as Iscove's Modified Dulbecco's Medium (IMDM) or Advanced Dulbecco's Modified Eagle's Medium (ADMEM); or a combination thereof.

Embodiment 3. The maturation medium of embodiment 1 or 2, wherein the additional carnitine or creatine is present in an amount of about 60-160 μM and/or wherein a total amount of carnitine or creatine present in the maturation medium is about 60-200 μM.

Embodiment 4. The maturation medium of any one of embodiments 1 to 3, wherein the additional T3 growth hormone is present in an amount of about 10-50 nM and/or wherein a total amount of T3 growth hormone present in the maturation medium is about 10-60 nM.

Embodiment 5. The maturation medium of any one of embodiments 1 to 4, wherein the one or more additional fatty acid comprises palmitic acid, oleic acid, linoleic acid, stearic acid, or a combination thereof.

Embodiment 6. The maturation medium of embodiment 5, wherein the maturation medium comprises a total amount of about 20-80 μM palmitic acid, about 20-80 μM oleic acid, and about 10-60 μM linoleic acid.

Embodiment 7. The maturation medium of any one of embodiments 1 to 6, wherein the maturation medium comprises the additional carnitine, the additional carnitine comprising L-carnitine, acetyl-L-carnitine, propionyl-L-carnitine, or a combination thereof.

Embodiment 8. The maturation medium of any one of embodiments 1 to 7, further comprising an additional sugar, such as fructose, galactose, or glucose.

Embodiment 9. The maturation medium of embodiment 8, wherein the additional sugar comprises glucose, such as about 2-6 mM glucose.

Embodiment 10. The maturation medium of any one of embodiments 1 to 9, further comprising an additional antioxidant, such as ascorbic acid (vitamin C), glutathione, lipoic acid, uric acid, a carotene, a tocopherol (vitamin E), and ubiquinol.

Embodiment 11. The maturation medium of any one of embodiments 1 to 10, further comprising ascorbic acid (vitamin C), such as about 0.1-1 mM ascorbic acid (vitamin C).

Embodiment 12. The maturation medium of any one of embodiments 1 to 11, further comprising a growth factor, such as IGF-1 or IGF-2.

Embodiment 13. The maturation medium of any one of embodiments 1 to 12, further comprising IGF-1 or IGF-2, for example, about 10-100 ng/mL.

Embodiment 14. The maturation medium of any one of embodiments 1 to 13, wherein the maturation medium does not include an extracellular matrix material and/or exogenous retinoic acid.

Embodiment 15. A method for maturing an early embryonic human heart organoid into a mature human heart organoid, the method comprising contacting the early embryonic human heart organoid with a maturation medium comprising: a cell growth medium containing a medium supplement, wherein the medium supplement comprises one or more fatty acid, triiodothyronine (T3) growth hormone, insulin, one or more antioxidant, a sugar, and carnitine; one or more additional fatty acid; an additional carnitine or creatine; and an additional T3 growth hormone.

Embodiment 16. The method of embodiment 15, wherein the cell growth medium comprises Roswell Park Memorial Institute (RPMI) medium; Dulbecco's Modified Eagle's Medium (DMEM); a derivative of DMEM such as Iscove's Modified Dulbecco's Medium (IMDM) or Advanced Dulbecco's Modified Eagle's Medium (ADMEM); or a combination thereof.

Embodiment 17. The method of embodiment 15 or 16, wherein the additional carnitine or creatine is present in an amount of about 60-160 μM and/or wherein a total amount of carnitine or creatine present in the maturation medium is about 60-200 PM.

Embodiment 18. The method of any one of embodiments 15 to 17, wherein the additional T3 growth hormone is present in an amount of about 10-50 nM and/or wherein a total amount of T3 growth hormone present in the maturation medium is about 10-60 nM.

Embodiment 19. The method of any one of embodiments 15 to 18, wherein the one or more additional fatty acid comprises palmitic acid, oleic acid, linoleic acid, stearic acid, of a combination thereof.

Embodiment 20. The method of embodiment 19, wherein the maturation medium comprises about 20-80 μM palmitic acid, about 20-80 μM oleic acid, and 10-60 μM linoleic acid.

Embodiment 21. The method of any one of embodiments 15 to 20, wherein the maturation medium comprises the additional carnitine, the additional carnitine comprising L-carnitine, acetyl-L-carnitine, propionyl-L-carnitine, or a combination thereof.

Embodiment 22. The method of any one of embodiments 15 to 21, wherein the maturation medium further comprises an additional sugar, such as fructose, galactose, or glucose.

Embodiment 23. The method of embodiment 22, wherein the additional sugar comprises glucose, such as about 2-6 mM glucose.

Embodiment 24. The method of any one of embodiments 15 to 23, wherein the maturation medium further comprises an additional antioxidant, such as ascorbic acid (vitamin C), glutathione, lipoic acid, uric acid, a carotene, a tocopherol (vitamin E), and ubiquinol.

Embodiment 25. The method of any one of embodiments 15 to 24, wherein the maturation medium further comprises ascorbic acid (vitamin C), such as about 0.1-1 mM ascorbic acid (vitamin C).

Embodiment 26. The method of any one of embodiments 15 to 25, wherein the maturation medium further comprises a growth factor, such as IGF-1 or IGF-2.

Embodiment 27. The method of any one of embodiments 15 to 26, wherein the maturation medium further comprises IGF-1 or IGF-2, for example, about 10-100 ng/mL.

Embodiment 28. The method of embodiment 15 or embodiment 25, wherein the early embryonic human heart organoid is formed from differentiation of human induced pluripotent stem cells (hiPSCs) and contacted with the maturation medium on day 20 following day zero of start of the differentiation of the hiPSCs.

Embodiment 29. The method of embodiment 15, wherein
from day 20 to day 26 the early embryonic human heart organoid is contacted with the maturation medium, wherein the maturation medium further comprises an additional antioxidant, such as ascorbic acid, an additional sugar, such as glucose, and a growth factor, such as IGF-1, and
from day 26 to day 30 the early embryonic heart organoid is contacted with the maturation medium, wherein the maturation medium further comprises an additional antioxidant, such as ascorbic acid, an additional sugar, such as glucose, and does not contain IGF-1; and
wherein the maturation medium is changed on day 26.

Embodiment 30. The method of embodiment 29, wherein a portion of the maturation medium from day 20 to day 26 contacts the early embryonic heart organoid from day 26 to day 30.

Embodiment 31. The method of embodiment 15, wherein the contacting occurs for 9, 10, 11, 12, or more than 12 days, preferably 10 days.

Embodiment 32. The method of any one of embodiments 15 to 31, wherein about every 48 hours the maturation medium contacting the early embryonic heart organoid is replaced with fresh maturation medium.

Embodiment 33. The method of any one of embodiments 15 to 32, wherein exogenous retinoic acid and/or an extracellular matrix material is not added.

Embodiment 34. The method of any one of embodiments 15 to 33, wherein the mature human heart organoid comprises one or more of the following:
(i) endogenous retinoic acid;
(ii) at least two cardiac chambers, one atrial and one ventricular;
(iii) a proepicardial organ; and
(iv) anterior-posterior heart tube patterning.

Embodiment 35. The method of any one of embodiments 15 to 34, wherein the mature human heart organoid is capable of beating.

Embodiment 36. A method for maturing an early embryonic human heart organoid into a mature human heart organoid, the method comprising contacting the early embryonic human heart organoid with one or more maturation mediums comprising:
(1) about 97% RPMI 1640 medium; about 2% medium supplement; about 1% penicillin streptomycin; about 52.5 μM palmitic acid in total; about 43.95 μM oleic acid in total; about 26 μM linoleic acid in total; about 132.2 μM L-carnitine in total, and about 33.01 nM T3 hormone in total; or
(2) about 97% RPMI 1640 medium having no glucose; about 2% medium supplement; about 1% penicillin streptomycin; about 52.5 μM palmitic acid in total; about 43.95 μM oleic acid in total; about 26 μM linoleic acid in total; about 132.2 μM L-carnitine in total, about 33.01 nM T3 hormone in total, about 0.4 mM ascorbic acid in total; and about 4 mM glucose in total; or
(3) about 97% RPMI 1640 medium having no glucose; about 2% medium supplement; about 1% penicillin streptomycin; about 52.5 μM palmitic acid in total; about 43.95 μM oleic acid in total; about 26 μM linoleic acid in total; about 132.2 μM L-carnitine in total, about 33.01 nM T3 hormone in total, about 0.4 mM ascorbic acid in total; about 4 mM glucose in total, and about 50 ng/mL IGF-1.

Embodiment 37. A mature human heart organoid produced by any one of the methods of embodiments 15-36.

Embodiment 38. The mature human heart organoid of embodiment 37, wherein the mature human heart organoid comprises one or more of the following:
(i) endogenous retinoic acid;
(ii) at least two cardiac chambers, one atrial and one ventricular;
(iii) a proepicardial organ; and
(iv) anterior-posterior heart tube patterning.

Embodiment 39. The mature human heart organoid of embodiment 37 or 38, wherein the mature human heart is capable of beating.

### REFERENCES

1. Mensah, G. A., Roth, G. A. & Fuster, V. The Global Burden of Cardiovascular Diseases and Risk Factors. J. Am. Coll. Cardiol. 74, 2529-2532 (2019).
2. Moreira, L. M. et al. Paracrine signalling by cardiac calcitonin controls atrial fibrogenesis and arrhythmia. Nature 587, 460-465 (2020).
3. Lee, J. et al. Activation of PDGF pathway links LMNA mutation to dilated cardiomyopathy. Nature 572, 335-340 (2019).
4. Lee, J. H., Protze, S. I., Laksman, Z., Backx, P. H. & Keller, G. M. Human Pluripotent Stem Cell-Derived Atrial and Ventricular Cardiomyocytes Develop from Distinct Mesoderm Populations. Cell Stem Cell 21, 179-194.e4 (2017).
5. Richards, D. J. et al. Human cardiac organoids for the modelling of myocardial infarction and drug cardiotoxicity. Nat. Biomed. Eng. 4, 446-462 (2020).
6. MacQueen, L. A. et al. A tissue-engineered scale model of the heart ventricle. Nat. Biomed. Eng. 2, 930-941 (2018).
7. Tiburcy, M. et al. Defined Engineered Human Myocardium With Advanced Maturation for Applications in Heart Failure Modeling and Repair. Circulation 135, 1832-1847 (2017).
8. Ingber, D. E. Human organs-on-chips for disease modelling, drug development and personalized medicine. Nat. Rev. Genet. 23, 467-491 (2022).
9. Gintant, G. et al. Use of Human Induced Pluripotent Stem Cell-Derived Cardiomyocytes in Preclinical Cancer Drug Cardiotoxicity Testing: A Scientific Statement From the American Heart Association. Circ. Res. 125, (2019).
10. Chen, G. et al. Chemically defined conditions for human iPSC derivation and culture. Nat. Methods 8, 424-429 (2011).
11. Xu, X. et al. Uncompensated mitochondrial oxidative stress underlies heart failure in an iPSC-derived model of congenital heart disease. Cell Stem Cell 29, 840-855.e7 (2022).
12. Theodoris, C. V. et al. Network-based screen in iPSC-derived cells reveals therapeutic candidate for heart valve disease. Science 371, eabd0724 (2021).
13. Clevers, H. Modeling Development and Disease with Organoids. Cell 165, 1586-1597 (2016).
14. Lancaster, M. A. & Knoblich, J. A. Organogenesis in a dish: Modeling development and disease using organoid technologies. Science 345, (2014).
15. Lewis-Israeli, Y. R. et al. Self-assembling human heart organoids for the modeling of cardiac development and congenital heart disease. Nat. Commun. 12, 5142 (2021).
16. Hofbauer, P. et al. Cardioids reveal self-organizing principles of human cardiogenesis. Cell 184, 3299-3317.e22 (2021).
17. Drakhlis, L. et al. Human heart-forming organoids recapitulate early heart and foregut development. Nat. Biotechnol. 39, 737-746 (2021).
18. Andersen, P. et al. Precardiac organoids form two heart fields via Bmp/Wnt signaling. Nat. Commun. 9, 3140 (2018).
19. Richards, D. J. et al. Inspiration from heart development: Biomimetic development of functional human cardiac organoids. Biomaterials 142, 112-123 (2017).
20. Feyen, D. A. M. et al. Metabolic Maturation Media Improve Physiological Function of Human iPSC-Derived Cardiomyocytes. Cell Rep. 32, 107925 (2020).
21. Correia, C. et al. Distinct carbon sources affect structural and functional maturation of cardiomyocytes derived from human pluripotent stem cells. Sci. Rep. 7, 8590 (2017).
22. Yang, X. et al. Tri-iodo-1-thyronine promotes the maturation of human cardiomyocytes-derived from induced pluripotent stem cells. J. Mol. Cell. Cardiol. 72, 296-304 (2014).
23. Yang, X. et al. Fatty Acids Enhance the Maturation of Cardiomyocytes Derived from Human Pluripotent Stem Cells. Stem Cell Rep. 13, 657-668 (2019).
24. Alvino, G. et al. Maternal and Fetal Fatty Acid Profile in Normal and Intrauterine Growth Restriction Pregnancies With and Without Preeclampsia. Pediatr. Res. 64, 615-620 (2008).
25. Longo, N., Frigeni, M. & Pasquali, M. Carnitine transport and fatty acid oxidation. Biochim. Biophys. Acta BBA—Mol. Cell Res. 1863, 2422-2435 (2016).
26. Oey, N. A. et al. Long-Chain Fatty Acid Oxidation during Early Human Development. Pediatr. Res. 57, 755-759 (2005).
27. Iruretagoyena, J. I. et al. Metabolic gene profile in early human fetal heart development. Mol. Hum. Reprod. 20, 690-700 (2014).
28. Chung, S. et al. Mitochondrial oxidative metabolism is required for the cardiac differentiation of stem cells. Nat. Clin. Pract. Cardiovasc. Med. 4, S60-S67 (2007).
29. Gaspar, J. A. et al. Unique Metabolic Features of Stem Cells, Cardiomyocytes, and Their Progenitors. Circ. Res. 114, 1346-1360 (2014).
30. Chung, S., Arrell, D. K., Faustino, R. S., Terzic, A. & Dzeja, P. P. Glycolytic network restructuring integral to the energetics of embryonic stem cell cardiac differentiation. J. Mol. Cell. Cardiol. 48, 725-734 (2010).
31. Klein, I. & Ojamaa, K. Thyroid Hormone and the Cardiovascular System. N. Engl. J. Med. 344, 501-509 (2001).
32. Chattergoon, N. N. Thyroid hormone signaling and consequences for cardiac development. J. Endocrinol. 242, T145-T160 (2019).
33. Marconi, A. M. et al. The impact of gestational age and fetal growth on the maternal-fetal glucose concentration difference. Obstet. Gynecol. 87, 937-942 (1996).
34. Bozzetti, P. et al. The relationship of maternal and fetal glucose concentrations in the human from midgestation until term. Metab.—Clin. Exp. 37, 358-363 (1988).
35. Kilby, M. D., Neary, R. H., Mackness, M. I. & Durrington, P. N. Fetal and Maternal Lipoprotein Metabolism in Human Pregnancy Complicated by Type I Diabetes Mellitus1. J. Clin. Endocrinol. Metab. 83, 1736-1741 (1998).
36. Sugamura, K. & Keaney, John F. Reactive oxygen species in cardiovascular disease. Free Radic. Biol. Med. 51, 978-992 (2011).
37. He, F. & Zuo, L. Redox Roles of Reactive Oxygen Species in Cardiovascular Diseases. Int. J. Mol. Sci. 16, 27770-27780 (2015).
38. Li, P. et al. IGF signaling directs ventricular cardiomyocyte proliferation during embryonic heart development. Development 138, 1795-1805 (2011).
39. Liu, J.-P., Baker, J., Perkins, A. S., Robertson, E. J. & Efstratiadis, A. Mice carrying null mutations of the genes encoding insulin-like growth factor I (Igf-1) and type 1 IGF receptor (Igf1r). Cell 75, 59-72 (1993).
40. Ren, J., Samson, W. K. & Sowers, J. R. Insulin-like growth factor I as a cardiac hormone: physiological and pathophysiological implications in heart disease. J. Mol. Cell. Cardiol. 31, 2049-2061 (1999).
41. Asp, M. et al. A Spatiotemporal Organ-Wide Gene Expression and Cell Atlas of the Developing Human Heart. Cell 179, 1647-1660.e19 (2019).
42. Cui, Y. et al. Single-Cell Transcriptome Analysis Maps the Developmental Track of the Human Heart. Cell Rep. 26, 1934-1950.e5 (2019).
43. Gollob, M. H. et al. Somatic Mutations in the Connexin 40 Gene (GJA5) in Atrial Fibrillation. N. Engl. J. Med. 354, 2677-2688 (2006).
44. Takasaki, A. et al. Sarcomere gene variants act as a genetic trigger underlying the development of left ventricular noncompaction. Pediatr. Res. 84, 733-742 (2018).
45. Martins, A. S. et al. In Vivo Analysis of Troponin C Knock-In (A8V) Mice. Circ. Cardiovasc. Genet. 8, 653-664 (2015).
46. Rosenfeld, G. E., Mercer, E. J., Mason, C. E. & Evans, T. Small heat shock proteins Hspb7 and Hspb12 regulate early steps of cardiac morphogenesis. Dev. Biol. 381, 389-400 (2013).
47. Wu, T. et al. HSPB7 is indispensable for heart development by modulating actin filament assembly. Proc. Natl. Acad. Sci. 114, 11956-11961 (2017).
48. Smith, C. L., Baek, S. T., Sung, C. Y. & Tallquist, M. D. Epicardial-Derived Cell Epithelial-to-Mesenchymal Transition and Fate Specification Require PDGF Receptor Signaling. Circ. Res. 108, e15-e26 (2011).
49. Mellgren, A. M. et al. Platelet-Derived Growth Factor Receptor R Signaling Is Required for Efficient Epicardial Cell Migration and Development of Two Distinct Coronary Vascular Smooth Muscle Cell Populations. Circ. Res. 103, 1393-1401 (2008).
50. Katz, T. C. et al. Distinct Compartments of the Proepicardial Organ Give Rise to Coronary Vascular Endothelial Cells. Dev. Cell 22, 639-650 (2012).
51. Kanisicak, O. et al. Genetic lineage tracing defines myofibroblast origin and function in the injured heart. Nat. Commun. 7, 12260 (2016).
52. Tandon, P., Miteva, Y. V., Kuchenbrod, L. M., Cristea, I. M. & Conlon, F. L. Tcf21 regulates the specification and maturation of proepicardial cells. Development 140, 2409-2421 (2013).
53. Knight-Schrijver, V. R. et al. A single-cell comparison of adult and fetal human epicardium defines the age-associated changes in epicardial activity. Nat. Cardiovasc. Res. 1, 1215-1229 (2022).
54. Meier, A. B. et al. Epicardioid single-cell genomics uncovers principles of human epicardium biology in heart development and disease. Nat. Biotechnol. 1-14 (2023) doi:10.1038/s41587-023-01718-7.
55. Moore, A. W., McInnes, L., Kreidberg, J., Hastie, N. D. & Schedl, A. YAC complementation shows a requirement for Wt1 in the development of epicardium, adrenal gland and throughout nephrogenesis. Development 126, 1845-1857 (1999).
56. Greulich, F., Farin, H. F., Schuster-Gossler, K. & Kispert, A. Tbx18 function in epicardial development. Cardiovasc. Res. 96, 476-483 (2012).
57. Di Paolo, G. et al. The phosphoprotein stathmin is essential for nerve growth factor-stimulated differentiation. J. Cell Biol. 133, 1383-1390 (1996).
58. Mori, N. & Morii, H. SCG10-related neuronal growth-associated proteins in neural development, plasticity, degeneration, and aging. J. Neurosci. Res. 70, 264-273 (2002).
59. Guerra San Juan, I. et al. Loss of mouse Stmn2 function causes motor neuropathy. Neuron 110, 1671-1688.e6 (2022).
60. Taupenot, L., Harper, K. L. & O'Connor, D. T. The Chromogranin-Secretogranin Family. N. Engl. J. Med. 348, 1134-1149 (2003).
61. Kim, H. J. et al. REST Regulates Non-Cell-Autonomous Neuronal Differentiation and Maturation of Neural Progenitor Cells via Secretogranin II. J. Neurosci. 35, 14872-14884 (2015).
62. Lan, M. S. & Breslin, M. B. Structure, expression, and biological function of INSM1 transcription factor in neuroendocrine differentiation. FASEB J. 23, 2024-2033 (2009).
63. Privratsky, J. R. & Newman, P. J. PECAM-1: regulator of endothelial junctional integrity. Cell Tissue Res. 355, 607-619 (2014).
64. Hirata, K. et al. Cloning of an Immunoglobulin Family Adhesion Molecule Selectively Expressed by Endothelial Cells*. J. Biol. Chem. 276, 16223-16231 (2001).
65. Downes, M. & Koopman, P. SOX18 and the Transcriptional Regulation of Blood Vessel Development. Trends Cardiovasc. Med. 11, 318-324 (2001).
66. Fong, G.-H., Rossant, J., Gertsenstein, M. & Breitman, M. L. Role of the Flt-1 receptor tyrosine kinase in regulating the assembly of vascular endothelium. Nature 376, 66-70 (1995).
67. Kang, I. S. et al. Characterization of human cardiac mesenchymal stromal cells and their extracellular vesicles comparing with human bone marrow derived mesenchymal stem cells. BMB Rep. 53, 118-123 (2020).
68. Eirin, A. et al. Comparative proteomic analysis of extracellular vesicles isolated from porcine adipose tissue-derived mesenchymal stem/stromal cells. Sci. Rep. 6, 36120 (2016).
69. Lincoln, J., Kist, R., Scherer, G. & Yutzey, K. E. Sox9 is required for precursor cell expansion and extracellular matrix organization during mouse heart valve development. Dev. Biol. 305, 120-132 (2007).
70. Li, Y., Lui, K. O. & Zhou, B. Reassessing endothelial-to-mesenchymal transition in cardiovascular diseases. Nat. Rev. Cardiol. 15, 445-456 (2018).
71. Schack, L. M. et al. Expression of CD24 in Human Bone Marrow-Derived Mesenchymal Stromal Cells Is Regulated by TGFβ3 and Induces a Myofibroblast-Like Genotype. Stem Cells Int. 2016, e1319578 (2015).
72. Garside, V. C. et al. SOX9 modulates the expression of key transcription factors required for heart valve development. Development 142, 4340-4350 (2015).
73. Walsh, E. C. & Stainier, D. Y. R. UDP-Glucose Dehydrogenase Required for Cardiac Valve Formation in Zebrafish. Science 293, 1670-1673 (2001).
74. Hyde, A. S. et al. UDP-glucose Dehydrogenase Polymorphisms from Patients with Congenital Heart Valve Defects Disrupt Enzyme Stability and Quaternary Assembly*. J. Biol. Chem. 287, 32708-32716 (2012).
75. Moskowitz, I. P. et al. Transcription factor genes Smad4 and Gata4 cooperatively regulate cardiac valve development. Proc. Natl. Acad. Sci. 108, 4006-4011 (2011).
76. Alfieri, C. M., Cheek, J., Chakraborty, S. & Yutzey, K. E. Wnt signaling in heart valve development and osteogenic gene induction. Dev. Biol. 338, 127-135 (2010).
77. Queen, R. et al. Spatial transcriptomics reveals novel genes during the remodelling of the embryonic human arterial valves. 2023.05.09.539950 Preprint at https://doi.org/10.1101/2023.05.09.539950 (2023).
78. Cheng, L. et al. Generation and characterization of cardiac valve endothelial-like cells from human pluripotent stem cells. Commun. Biol. 4, 1-15 (2021).
79. Zhang, Q. et al. Designing a Green Fluorogenic Protease Reporter by Flipping a Beta Strand of GFP for Imaging Apoptosis in Animals. J. Am. Chem. Soc. 141, 4526-4530 (2019).
80. Hortells, L. et al. A specialized population of Periostin-expressing cardiac fibroblasts contributes to postnatal cardiomyocyte maturation and innervation. Proc. Natl. Acad. Sci. 117, 21469-21479 (2020).
81. Ryan, A. K. et al. Pitx2 determines left-right asymmetry of internal organs in vertebrates. Nature 394, 545-551 (1998).
82. Ai, D. et al. Pitx2 regulates cardiac left-right asymmetry by patterning second cardiac lineage-derived myocardium. Dev. Biol. 296, 437-449 (2006).
83. Ocaña, O. H. et al. A right-handed signalling pathway drives heart looping in vertebrates. Nature 549, 86-90 (2017).
84. Meno, C. et al. lefty-1 Is Required for Left-Right Determination as a Regulator of lefty-2 and nodal. Cell 94, 287-297 (1998).
85. Uxa, S. et al. Ki-67 gene expression. Cell Death Differ. 28, 3357-3370 (2021).
86. Whitfield, M. L., George, L. K., Grant, G. D. & Perou, C. M. Common markers of proliferation. Nat. Rev. Cancer 6, 99-106 (2006).
87. Nigg, E. A. Mitotic kinases as regulators of cell division and its checkpoints. Nat. Rev. Mol. Cell Biol. 2, 21-32 (2001).
88. Kelly, R. G., Buckingham, M. E. & Moorman, A. F. Heart fields and cardiac morphogenesis. Cold Spring Harb. Perspect. Med. 4, a015750 (2014).
89. Cai, C.-L. et al. Isl1 Identifies a Cardiac Progenitor Population that Proliferates Prior to Differentiation and Contributes a Majority of Cells to the Heart. Dev. Cell 5, 877-889 (2003).
90. McFadden, D. G. et al. The Hand1 and Hand2 transcription factors regulate expansion of the embryonic cardiac ventricles in a gene dosage-dependent manner. Development 132, 189-201 (2005).
91. Vrljicak, P. et al. Twist1 Transcriptional Targets in the Developing Atrio-Ventricular Canal of the Mouse. PLOS ONE 7, e40815 (2012).
92. Schleiffarth, J. R. et al. Wnt5a Is Required for Cardiac Outflow Tract Septation in Mice. Pediatr. Res. 61, 386-391 (2007).
93. Gessert, S. & Kuhl, M. The Multiple Phases and Faces of Wnt Signaling During Cardiac Differentiation and Development. Circ. Res. 107, 186-199 (2010).
94. Hofbauer, P. et al. Cardioids reveal self-organizing principles of human cardiogenesis. bioRxiv 2020.07.06.189431 (2020) doi:10.1101/2020.07.06.189431.
95. Lopaschuk, G. D. & Jaswal, J. S. Energy metabolic phenotype of the cardiomyocyte during development, differentiation, and postnatal maturation. J. Cardiovasc. Pharmacol. 56, 130-140 (2010).
96. Piquereau, J. & Ventura-Clapier, R. Maturation of Cardiac Energy Metabolism During Perinatal Development. Front. Physiol. 9, 959 (2018).
97. Barger, P. M. & Kelly, D. P. PPAR signaling in the control of cardiac energy metabolism. Trends Cardiovasc. Med. 10, 238-245 (2000).
98. LeBleu, V. S. et al. PGC-la mediates mitochondrial biogenesis and oxidative phosphorylation in cancer cells to promote metastasis. Nat. Cell Biol. 16, 992-1003 (2014).
99. Jogl, G. & Tong, L. Crystal Structure of Carnitine Acetyltransferase and Implications for the Catalytic Mechanism and Fatty Acid Transport. Cell 112, 113-122 (2003).
100. Weis, B. C., Cowan, A. T., Brown, N., Foster, D. W. & McGarry, J. D. Use of a selective inhibitor of liver carnitine palmitoyltransferase I (CPT I) allows quantification of its contribution to total CPT I activity in rat heart. Evidence that the dominant cardiac CPT I isoform is identical to the skeletal muscle enzyme. J. Biol. Chem. 269, 26443-26448 (1994).
101. Luo, W., Pant, G., Bhavnasi, Y. K., Blanchard, S. G., Jr & Brouwer, C. Pathview Web: user friendly pathway visualization and data integration. Nucleic Acids Res. 45, W501-W508 (2017).
102. Luo, W. & Brouwer, C. Pathview: an R/Bioconductor package for pathway-based data integration and visualization. Bioinformatics 29, 1830-1831 (2013).
103. Bers, D. M. Cardiac excitation-contraction coupling. Nature 415, 198-205 (2002).
104. Giudicessi, J. R. & Ackerman, M. J. Potassium-channel mutations and cardiac arrhythmias-diagnosis and therapy. Nat. Rev. Cardiol. 9, 319-332 (2012).
105. Leblanc, N. & Hume, J. R. Sodium current-induced release of calcium from cardiac sarcoplasmic reticulum. Science 248, 372-376 (1990).
106. Pennisi, D. J., Rentschler, S., Gourdie, R. G., Fishman, G. I. & Mikawa, T. Induction and patterning of the cardiac conduction system. Int. J. Dev. Biol. 46, 765-775 (2004).
107. van Weerd, J. H. & Christoffels, V. M. The formation and function of the cardiac conduction system. Development 143, 197-210 (2016).
108. Schats, R., Jansen, C. A. & Wladimiroff, J. W. Embryonic heart activity: appearance and development in early human pregnancy. Br. J. Obstet. Gynaecol. 97, 989-994 (1990).
109. Manner, J. When Does the Human Embryonic Heart Start Beating? A Review of Contemporary and Historical Sources of Knowledge about the Onset of Blood Circulation in Man. J. Cardiovasc. Dev. Dis. 9, 187 (2022).
110. Warmke, J. W. & Ganetzky, B. A family of potassium channel genes related to eag in Drosophila and mammals. Proc. Natl. Acad. Sci. U.S.A 91, 3438-3442 (1994).
111. Sanguinetti, M. C. & Tristani-Firouzi, M. hERG potassium channels and cardiac arrhythmia. Nature 440, 463-469 (2006).
112. Carss, K. J. et al. Using human genetics to improve safety assessment of therapeutics. Nat. Rev. Drug Discov. 1-18 (2022) doi:10.1038/s41573-022-00561-w.
113. Holbrook, M., Malik, M., Shah, R. R. & Valentin, J.-P. Drug induced shortening of the QT/QTc interval: An emerging safety issue warranting further modelling and evaluation in drug research and development? J. Pharmacol. Toxicol. Methods 59, 21-28 (2009).
114. Sanguinetti, M. C., Jiang, C., Curran, M. E. & Keating, M. T. A mechanistic link between an inherited and an acquired cardiac arrhythmia: HERG encodes the IKr potassium channel. Cell 81, 299-307 (1995).
115. Curran, M. E. et al. A molecular basis for cardiac arrhythmia: HERG mutations cause long QT syndrome. Cell 80, 795-803 (1995).
116. De Bruin, M. L., Pettersson, M., Meyboom, R. H. B., Hoes, A. W. & Leufkens, H. G. M. Anti-HERG activity and the risk of drug-induced arrhythmias and sudden death. Eur. Heart J. 26, 590-597 (2005).
117. Fenichel, R. R. et al. Drug-induced torsades de pointes and implications for drug development. J. Cardiovasc. Electrophysiol. 15, 475-495 (2004).
118. Ahlquist, R. P. A study of the adrenotropic receptors. Am. J. Physiol.-Leg. Content 153, 586-600 (1948).
119. Rockman, H. A., Koch, W. J. & Lefkowitz, R. J. Seven-transmembrane-spanning receptors and heart function. Nature 415, 206-212 (2002).
120. Logothetis, D. E., Kurachi, Y., Galper, J., Neer, E. J. & Clapham, D. E. The βγ subunits of GTP-binding proteins activate the muscarinic K+ channel in heart. Nature 325, 321-326 (1987).
121. Florea, V. G. & Cohn, J. N. The Autonomic Nervous System and Heart Failure. Circ. Res. 114, 1815-1826 (2014).
122. Lymperopoulos, A., Rengo, G. & Koch, W. J. Adrenergic Nervous System in Heart Failure. Circ. Res. 113, 739-753 (2013).
123. Uhl\tilde{e}n, M. et al. Proteomics. Tissue-based map of the human proteome. Science 347, 1260419 (2015).
124. Karlsson, M. et al. A single-cell type transcriptomics map of human tissues. Sci. Adv. 7, eabh2169 (2021).
125. Lohse, M. J., Engelhardt, S. & Eschenhagen, T. What Is the Role of β-Adrenergic Signaling in Heart Failure?Circ. Res. 93, 896-906 (2003).
126. Brette, F. & Orchard, C. T-tubule function in mammalian cardiac myocytes. Circ. Res. 92, 1182-1192 (2003).
127. Ibrahim, M., Gorelik, J., Yacoub, M. H. & Terracciano, C. M. The structure and function of cardiac t-tubules in health and disease. Proc. Biol. Sci. 278, 2714-2723 (2011).
128. Huang, Y. et al. Optical Coherence Tomography Detects Necrotic Regions and Volumetrically Quantifies Multicellular Tumor Spheroids. Cancer Res. 77, 6011-6020 (2017).
129. Ming, Y. et al. Longitudinal morphological and functional characterization of human heart organoids using optical coherence tomography. Biosens. Bioelectron. 207, 114136 (2022).
130. Rhinn, M. & Dollé, P. Retinoic acid signalling during development. Development 139, 843-858 (2012).
131. Wiesinger, A., Boink, G. J. J., Christoffels, V. M. & Devalla, H. D. Retinoic acid signaling in heart development: Application in the differentiation of cardiovascular lineages from human pluripotent stem cells. Stem Cell Rep. 16, 2589-2606 (2021).
132. Niederreither, K. & Doll6, P. Retinoic acid in development: towards an integrated view. Nat. Rev. Genet. 9, 541-553 (2008).
133. Duester, G. Retinoic Acid Synthesis and Signaling during Early Organogenesis. Cell 134, 921-931 (2008).
134. Cai, C.-L. et al. A myocardial lineage derives from Tbx18 epicardial cells. Nature 454, 104-108 (2008).
135. Lupu, I.-E., Redpath, A. N. & Smart, N. Spatiotemporal Analysis Reveals Overlap of Key Proepicardial Markers in the Developing Murine Heart. Stem Cell Rep. 14, 770-787 (2020).
136. Lemon, L. S. et al. Ondansetron use in the first trimester of pregnancy and the risk of neonatal ventricular septal defect. Int. J. Epidemiol. 49, 648-656 (2020).
137. Lee, J. et al. In vitro generation of functional murine heart organoids via FGF4 and extracellular matrix. Nat. Commun. 11, 4283 (2020).
138. Liang, P. et al. Drug Screening Using a Library of Human Induced Pluripotent Stem Cell-Derived Cardiomyocytes Reveals Disease-Specific Patterns of Cardiotoxicity. Circulation 127, 1677-1691 (2013).
139. Kopljar, I. et al. Development of a Human iPSC Cardiomyocyte-Based Scoring System for Cardiac Hazard Identification in Early Drug Safety De-risking. Stem Cell Rep. 11, 1365-1377 (2018).
140. Kodo, K. et al. iPSC-derived cardiomyocytes reveal abnormal TGF-β signalling in left ventricular non-compaction cardiomyopathy. Nat. Cell Biol. 18, 1031-1042 (2016).
141. Kolwicz, S. C., Purohit, S. & Tian, R. Cardiac Metabolism and its Interactions With Contraction, Growth, and Survival of Cardiomyocytes. Circ. Res. 113, 603-616 (2013).
142. Mills, R. J. et al. Functional screening in human cardiac organoids reveals a metabolic mechanism for cardiomyocyte cell cycle arrest. Proc. Natl. Acad. Sci. 114, E8372-E8381 (2017).
143. Duenas, A., Aranega, A. E. & Franco, D. More than Just a Simple Cardiac Envelope; Cellular Contributions of the Epicardium. Front. Cell Dev. Biol. 5, (2017).
144. Furtado, M. B., Nim, H. T., Boyd, S. E. & Rosenthal, N. A. View from the heart: cardiac fibroblasts in development, scarring and regeneration. Development 143, 387-397 (2016).
145. Christoffels, V. M. & Moorman, A. F. M. Development of the Cardiac Conduction System. Circ. Arrhythm. Electrophysiol. 2, 195-207 (2009).
146. Ribeiro, A. J. S. et al. Contractility of single cardiomyocytes differentiated from pluripotent stem cells depends on physiological shape and substrate stiffness. Proc. Natl. Acad. Sci. 112, 12705-12710 (2015).
147. Pioner, J. M. et al. Isolation and Mechanical Measurements of Myofibrils from Human Induced Pluripotent Stem Cell-Derived Cardiomyocytes. Stem Cell Rep. 6, 885-896 (2016).
148. Goversen, B., van der Heyden, M. A. G., van Veen, T. A. B. & de Boer, T. P. The immature electrophysiological phenotype of iPSC-CMs still hampers in vitro drug screening: Special focus on IK1. Pharmacol. Ther. 183, 127-136 (2018).
149. Hibino, H. et al. Inwardly Rectifying Potassium Channels: Their Structure, Function, and Physiological Roles. Physiol. Rev. 90, 291-366 (2010).
150. Gittenberger-de Groot, A. C., Bartelings, M. M., Deruiter, M. C. & Poelmann, R. E. Basics of Cardiac Development for the Understanding of Congenital Heart Malformations. Pediatr. Res. 57, 169-176 (2005).
151. Hikspoors, J. P. J. M. et al. A pictorial account of the human embryonic heart between 3.5 and 8 weeks of development. Commun. Biol. 5, 1-22 (2022).
152. Meilhac, S. M. & Buckingham, M. E. The deployment of cell lineages that form the mammalian heart. Nat. Rev. Cardiol. 15, 705-724 (2018).
153. Dekkers, J. F. et al. High-resolution 3D imaging of fixed and cleared organoids. Nat. Protoc. 14, 1756-1771 (2019).
154. Chen, E. Y. et al. Enrichr: interactive and collaborative HTML5 gene list enrichment analysis tool. BMC Bioinformatics 14, 128 (2013).
155. Kuleshov, M. V. et al. Enrichr: a comprehensive gene set enrichment analysis web server 2016 update. Nucleic Acids Res. 44, W90-97 (2016).
156. Xie, Z. et al. Gene Set Knowledge Discovery with Enrichr. Curr. Protoc. 1, e90 (2021).
157. Dimitrov, D. et al. Comparison of methods and resources for cell-cell communication inference from single-cell RNA-Seq data. Nat. Commun. 13, 3224 (2022).
158. Zhou, J. et al. The Expression of Cytokine Profiles and Related Receptors in Idiopathic Inflammatory Myopathies. Front. Pharmacol. 13, 852055 (2022).
159. Hao, Y. et al. Integrated analysis of multimodal single-cell data. Cell 184, 3573-3587.e29 (2021).
160. Turei, D. et al. Integrated intra- and intercellular signaling knowledge for multicellular omics analysis. Mol. Syst. Biol. 17, e9923 (2021).
161. Ramilowski, J. A. et al. A draft network of ligand-receptor-mediated multicellular signalling in human. Nat. Commun. 6, 7866 (2015).
162. Hondeghem, L. M., Carlsson, L. & Duker, G. Instability and Triangulation of the Action Potential Predict Serious Proarrhythmia, but Action Potential Duration Prolongation Is Antiarrhythmic. Circulation 103, 2004-2013 (2001).

What is claimed is:

1. A maturation medium comprising:
a cell growth medium containing a medium supplement, wherein the medium supplement comprises one or more fatty acid, triiodothyronine (T3) growth hormone, insulin, one or more antioxidant, a sugar, and carnitine;
one or more additional fatty acid;
an additional carnitine or creatine; and
an additional T3 growth hormone.

2. The maturation medium of claim 1, wherein the cell growth medium comprises Roswell Park Memorial Institute (RPMI) medium; Dulbecco's Modified Eagle's Medium (DMEM); a derivative of DMEM such as Iscove's Modified Dulbecco's Medium (IMDM) or Advanced Dulbecco's Modified Eagle's Medium (ADMEM); or a combination thereof.

3. The maturation medium of claim 1, wherein the additional carnitine or creatine is present in an amount of about 60-160 μM and/or wherein a total amount of carnitine or creatine present in the maturation medium is about 60-200 μM.

4. The maturation medium of claim 1, wherein the additional T3 growth hormone is present in an amount of about 10-50 nM and/or wherein a total amount of T3 growth hormone present in the maturation medium is about 10-60 nM.

5. The maturation medium of claim 1, wherein the one or more additional fatty acid comprises palmitic acid, oleic acid, linoleic acid, stearic acid, or a combination thereof.

6. The maturation medium of claim 5, wherein the maturation medium comprises a total amount of about 20-80 μM palmitic acid, about 20-80 μM oleic acid, and about 10-60 μM linoleic acid.

7. The maturation medium of claim 1, wherein the maturation medium comprises the additional carnitine, the additional carnitine comprising L-carnitine, acetyl-L-carnitine, propionyl-L-carnitine, or a combination thereof.

8. The maturation medium of claim 1, further comprising an additional sugar, such as fructose, galactose, or glucose.

9. The maturation medium of claim 8, wherein the additional sugar comprises glucose, such as about 2-6 mM glucose.

10. The maturation medium of claim 1, further comprising an additional antioxidant, such as ascorbic acid (vitamin C), glutathione, lipoic acid, uric acid, a carotene, a tocopherol (vitamin E), and ubiquinol.

11. The maturation medium of claim 1, further comprising ascorbic acid (vitamin C), such as about 0.1-1 mM ascorbic acid (vitamin C).

12. The maturation medium of claim 1, further comprising a growth factor, such as IGF-1 or IGF-2.

13. The maturation medium of claim 1, further comprising IGF-1 or IGF-2, for example, about 10-100 ng/mL.

14. The maturation medium of claim 1, wherein the maturation medium does not include an extracellular matrix material and/or exogenous retinoic acid.

15. A method for maturing an early embryonic human heart organoid into a mature human heart organoid, the method comprising contacting the early embryonic human heart organoid with a maturation medium comprising:
a cell growth medium containing a medium supplement, wherein the medium supplement comprises one or more fatty acid, triiodothyronine (T3) growth hormone, insulin, one or more antioxidant, a sugar, and carnitine;
one or more additional fatty acid;
an additional carnitine or creatine; and
an additional T3 growth hormone.

16. The method of claim 15, wherein the cell growth medium comprises Roswell Park Memorial Institute (RPMI) medium; Dulbecco's Modified Eagle's Medium (DMEM); a derivative of DMEM such as Iscove's Modified Dulbecco's Medium (IMDM) or Advanced Dulbecco's Modified Eagle's Medium (ADMEM); or a combination thereof.

17. The method of claim 15, wherein the additional carnitine or creatine is present in an amount of about 60-160 μM and/or wherein a total amount of carnitine or creatine present in the maturation medium is about 60-200 μM.

18. The method of claim 15, wherein the additional T3 growth hormone is present in an amount of about 10-50 nM and/or wherein a total amount of T3 growth hormone present in the maturation medium is about 10-60 nM.

19. The method of claim 15, wherein the one or more additional fatty acid comprises palmitic acid, oleic acid, linoleic acid, stearic acid, of a combination thereof.

20. The method of claim 19, wherein the maturation medium comprises about 20-80 μM palmitic acid, about 20-80 μM oleic acid, and 10-60 μM linoleic acid.

21. The method of claim 15, wherein the maturation medium comprises the additional carnitine, the additional carnitine comprising L-carnitine, acetyl-L-carnitine, propionyl-L-carnitine, or a combination thereof.

22. The method of claim 15, wherein the maturation medium further comprises an additional sugar, such as fructose, galactose, or glucose.

23. The method of claim 22, wherein the additional sugar comprises glucose, such as about 2-6 mM glucose.

24. The method of claim 15, wherein the maturation medium further comprises an additional antioxidant, such as ascorbic acid (vitamin C), glutathione, lipoic acid, uric acid, a carotene, a tocopherol (vitamin E), and ubiquinol.

25. The method of claim 15, wherein the maturation medium further comprises ascorbic acid (vitamin C), such as about 0.1-1 mM ascorbic acid (vitamin C).

26. The method of claim 15, wherein the maturation medium further comprises a growth factor, such as IGF-1 or IGF-2.

27. The method of claim 15, wherein the maturation medium further comprises IGF-1 or IGF-2, for example, about 10-100 ng/mL.

28. The method of claim 15, wherein the early embryonic human heart organoid is formed from differentiation of human induced pluripotent stem cells (hiPSCs) and contacted with the maturation medium on day 20 following day zero of start of the differentiation of the hiPSCs.

29. The method of claim 15, wherein
from day 20 to day 26 the early embryonic human heart organoid is contacted with the maturation medium, wherein the maturation medium further comprises an additional antioxidant, such as ascorbic acid, an additional sugar, such as glucose, and a growth factor, such as IGF-1, and
from day 26 to day 30 the early embryonic heart organoid is contacted with the maturation medium, wherein the maturation medium further comprises an additional antioxidant, such as ascorbic acid, an additional sugar, such as glucose, and does not contain IGF-1; and
wherein the maturation medium is changed on day 26.

30. The method of claim 29, wherein a portion of the maturation medium from day 20 to day 26 contacts the early embryonic heart organoid from day 26 to day 30.

31. The method of claim 15, wherein the contacting occurs for 9, 10, 11, 12, or more than 12 days, preferably 10 days.

32. The method of claim 15, wherein about every 48 hours the maturation medium contacting the early embryonic heart organoid is replaced with fresh maturation medium.

33. The method of claim 15, wherein exogenous retinoic acid and/or an extracellular matrix material is not added.

34. The method of claim 15, wherein the mature human heart organoid comprises one or more of the following:
(v) endogenous retinoic acid;
(vi) at least two cardiac chambers, one atrial and one ventricular;
(vii) a proepicardial organ; and
(viii) anterior-posterior heart tube patterning.

35. The method of claim 15, wherein the mature human heart organoid is capable of beating.

36. A method for maturing an early embryonic human heart organoid into a mature human heart organoid, the method comprising contacting the early embryonic human heart organoid with one or more maturation mediums comprising:
(1) about 97% RPMI 1640 medium; about 2% medium supplement; about 1% penicillin streptomycin; about 52.5 μM palmitic acid in total; about 43.95 μM oleic acid in total; about 26 μM linoleic acid in total; about 132.2 μM L-carnitine in total, and about 33.01 nM T3 hormone in total; or
(2) about 97% RPMI 1640 medium having no glucose; about 2% medium supplement; about 1% penicillin streptomycin; about 52.5 μM palmitic acid in total; about 43.95 μM oleic acid in total; about 26 μM linoleic acid in total; about 132.2 μM L-carnitine in total, about 33.01 nM T3 hormone in total, about 0.4 mM ascorbic acid in total; and about 4 mM glucose in total; or
(3) about 97% RPMI 1640 medium having no glucose; about 2% medium supplement; about 1% penicillin streptomycin; about 52.5 μM palmitic acid in total; about 43.95 μM oleic acid in total; about 26 μM linoleic acid in total; about 132.2 μM L-carnitine in total, about 33.01 nM T3 hormone in total, about 0.4 mM ascorbic acid in total; about 4 mM glucose in total, and about 50 ng/mL IGF-1.

37. A mature human heart organoid produced by the method of claim 15.

38. The mature human heart organoid of claim 37, wherein the mature human heart organoid comprises one or more of the following:
(v) endogenous retinoic acid;
(vi) at least two cardiac chambers, one atrial and one ventricular;
(vii) a proepicardial organ; and
(viii) anterior-posterior heart tube patterning.

39. The mature human heart organoid of claim 37, wherein the mature human heart is capable of beating.
